(19) United States
(12) Patent Application Publication
(10) Pub. No.: US 2019/0038611 A1
(43) Pub. Date: Feb. 7, 2019

(54) Novel CYP34A-Specific Inhibitors and Methods of Using Same

(71) Applicant: THE REGENTS OF THE UNIVERSITY OF CALIFORNIA, Oakland, CA (US)

(72) Inventor: Irina Sevrioukova, Irvine, CA (US)

(21) Appl. No.: 15/757,716

(22) PCT Filed: Sep. 9, 2016
(86) PCT No.: PCT/US16/50944

§ 371 Date: Mar. 6, 2018

Related U.S. Application Data
(60) Provisional application No. 62/215,997, filed on Sep. 9, 2015.

(51) Int. Cl. A61K 31/4439; A61K 31/404; A61K 31/27; A61K 45/06; C07C 271/20; C07D 401/12; C07D 209/44; C07D 213/40

(52) U.S. Cl. CPC A61K 31/4439; A61K 31/404; A61K 31/27; C07D 213/40; C07C 271/20; C07D 401/12; C07D 209/44; A61K 45/06

(57) ABSTRACT

The present invention includes novel compositions inhibiting CYP3A4. The present invention further includes a novel method of inhibiting CYP3A4 in a subject in need thereof, the method comprising administering to the subject an effective amount of a compound of the invention. In one embodiment, the subject is further administered at least one additional therapeutic agent.

### CROSS-REFERENCE TO RELATED APPLICATIONS

This application claims priority to U.S. Provisional Application Ser. No. 62/215,997, filed Sep. 9, 2015, the contents of which are incorporated by reference herein in its entirety.

### REFERENCE TO GOVERNMENT GRANT

This invention was made with government support under grants GM57353 and ES025767 awarded by the National Institutes of Health and grant MRPI143226 awarded by the University of California's Center for Antiviral Drug Discovery. The Government has certain rights in the invention.

### BACKGROUND OF THE INVENTION

Cytochrome P450 3A4 (CYP3A4) is the most abundant and clinically relevant xenobiotic-metabolizing enzyme in humans. CYP3A4 catalyzes diverse oxidative reactions and is involved in metabolism of endogenous steroids, the majority of administered drugs and other foreign molecules, such as environmental pollutants, carcinogens, pesticides and insecticides (Guengerich and Shimada, 1991, Chem Res Toxicol 4:391-407; Li et al., 1995, Toxicology 104:1-8; Guengerich, 1999, Annu Rev Pharmacol Toxicol 39:1-17; Mehmood et al., 1997, Chemosphere 34:2281-91; Chae et al., 1993, Cancer Res 53:2028-34; Hodgson, 2003, J Biochem Mol Toxicol 17:201-6; Mehmood et al., 1996, Environ Toxicol Pharmacol 2:397-401; Mehmood et al., 1996, Chemosphere, 33:759-69). CYP3A4 has a large and malleable active site that accommodates a wide variety of molecules, some of which could inhibit or enhance metabolism. In vivo, CYP3A4 inhibition has a greater impact on human health, as it can lead to xenobiotic-induced toxicity and drug-drug interactions, one of the reasons for late-stage clinical trial failures and withdrawal of marketed pharmaceuticals.

However, carefully controlled CYP3A4 inhibition can be beneficial and is currently exploited in the treatment of HIV infection. To date, two CYP3A4 inhibitors, ritonavir and cobicistat are marketed as pharmacoenhancers for the HIV-I protease inhibitors that serve as substrates for CYP3A4 (Kempf et al., 1997, Antibicrom Agents Chemother 41:654-60; Xu and Desai et al., 2009, Curr Opin Investig Drugs 10:775-86; Mathias et al., 2010, Clin Pharmacol 87:322-9; Xu et al., 2010, ACS Med Chem Lett 1:209-13; Xu et al., 2014, Bioorg Med Chem Lett, 24:995-9). Both drugs were developed based on chemical structure/activity relationships studies rather than the CYP3A4 crystal structure. Moreover, ritonavir was originally designed to inhibit the HIV-1 protease (Kempf et al., 1995, Proc Natl Acad Sci USA 92:2484-8), whereas its ability to potently inactivate CYP3A4 was coincidental and discovered later (Kempf et al., 1997, Antibicrom Agents Chemother 41:654-60). Cobicistat is a derivative of ritonavir that lacks the backbone hydroxyl group, critical for binding to catalytic Asp25 and Asp225 of HIV-1 protease, and has a morpholine ring instead of the valine side group. As a result, cobicistat is not an anti-HIV protease inhibitor so its prolonged usage does not promote the development of drug-resistant HIV strains. Also, this pharmaceutical has fewer side effects and better physico-chemical properties, and is comparable but not superior to ritonavir in terms of inhibitory potency for CYP3A4 (Xu et al., 2010, ACS Med Chem Left 1:209-13).

There is no general agreement on the CYP3A4 inhibitory mechanism of ritonavir. Several groups suggested that ritonavir is a mechanism-based inactivator (Koudriakova et al., 1998, Drug Metab Dispos 26:552-61; von Moltke et al., 2000, Eur J Clin Pharmacol 56:259-61; Ernest et al., 2005, J Pharmacol Exp Ther 312:583-91; Linn et al., 2013, Drug Metab Dispos 41:1813-24; Rock et al., 2014, Mol Pharmacol 665-74), whereas others argued against the mechanism-based CYP3A4 inhibition (Sekiguchi et al., 2009, Drug Metab Pharmacokinet, 24:500-10) and concluded that ritonavir acts as a competitive (Iribarne et al., 1998, Drug Metab Dispos 26:257-60) or a mixed competitive-noncompetitive CYP3A4 inactivator 9 Eagling et al., 1997, Br J Clin Pharmacol 44:190-4; Kumar et al., 1996, J Pharmacol Exp Ther 277:423-31; Zalma et al., 2000, Biol Psychiatry 47:655-61). Data on highly purified recombinant CYP3A4 do not support the mechanism-based or competitive type of inhibition as a predominant inactivation pathway and rather suggest that ritonavir inactivates CYP3A4 via strong thiazole nitrogen coordination to both ferric and ferrous CYP3A4 which decreases the heme redox potential and impedes electron transfer from the redox partner, cytochrome P450 reductase (Sevrioukova and Poulos, 2010, Proc Natl Acad Sci USA 107:18422-7). This conclusion is well supported by the X-ray structures of CYP3A4 complexed with ritonavir and 10 ritonavir analogs (Sevrioukova and Poulos, 2010, Proc Natl Acad Sci USA 107:18422-7; Sevrioukova and Poulos, 2012, Arch biochem Biophys 520:108-16; Sevrioukova and Poulos, 2013, J Med Chem 56:3733-41; Sevrioukova and Poulos, 2013, Biochemistry 52:4474-81).

Previous 3D-pharmacophores for drug metabolism prediction were built based on P450 homology and quantitative structure-activity relationship (QSAR) modeling because the X-ray structure of CYP3A4 was not available at that time (Ekins et al., 1999, J Pharmacol Exp Ther 290:429-38; Ekins et al., 1999, J Pharmacol Exp Ther 291:424-33; Ekins et al., 2003, Trends Pharmacol Sci 24:161-6; Riley et al., 2001, Pharm Res 18:652-5). In addition, there is no structure-based inhibitor pharmacophore that could assist in identification and early elimination of potential CYP3A4 inactivators during development of drugs and other chemicals relevant to public health.

There is a need in the art to identify novel compounds which inactivate CYP3A4. The present invention fulfills this need.

### SUMMARY OF THE INVENTION

The invention includes a compound of Formula (I):

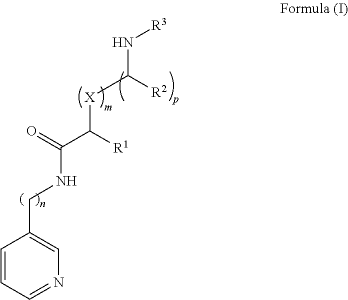

wherein in Formula (I):

R¹ and R² are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, substitued -(C₁-C₆)alkyl-heteroaryl, and N(R⁴)(R⁵);

R³ is selected from the group consisting of H, -C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted - (C₁-C₆)alkyl-phenyl, -(C₁-C₆)alkyl-carbocyclic, -(C₁-C₆)alkyl-heteroaryl, and substituted -(C₁-C₆)alkyl-heteroaryl and —C(═O)R⁶, wherein the alkyl group is optionally substituted;

R⁴ and R⁵ are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substitued-(C₁-C₆)alkyl-heteroaryl;

R⁶ is selected from the group consisting of phenyl, substituted phenyl, -(C₁-C₆)alkyl-phenyl, substitued -(C₁-C₆) alkyl-phenyl, -(C₁-C₆)alkyl-heteroaryl, substitued -(C₁-C₆)alkyl-heteroarly, and OW;

R⁷ is selected from the group consisting of H, -C₁-C₆ alkyl, substituted C₁-C₆ alkyl, aryl, cycloalkyl phenyl, substituted phenyl, heteroaryl, substituted heteroaryl, heterocyclyl, substituted heterocyclyl, -(C₁-C₆)alkyl-phenyl, substituted -(C₁-C₆)alkyl-aryl, substituted -(C₁-C₆)alkyl-aryl, -(C₁-C₆)alkyl-carbocyclic, -(C₁-C₆)alkyl-heteroaryl, substitued -(C₁-C₆)alkyl-heteroaryl;

each occurrence of X is independently selected from the group consisting of CH₂, NH, S, and O;

m is an integer from 0 to 3;

n is an integer from 0 to 3; and

p is an integer from 0 to 1;

a salt or solvate, and any combinations thereof.

In one embodiment, R¹ is selected from the group consisting of H, benzyl, and aniline.

In one embodiment, R² is selected from the group consisting of H, -(C₁-C₆)alkyl-carbocyclic, -(C₁-C₆)alkyl-phenyl, -(C₁-C₆)alkyl-substituted phenyl, -(C₁-C₆)alkyl-heteroaryl, and -(C₁-C₆)alkyl-substituted heteroaryl.

In one embodiment, R⁷ is -C₁-C₆ alkyl.

In one embodiment, m is 1. In another embodiment, n is 1.

In one embodiment, X is S.

In one embodiment, the compound is selected from the group consisting of:

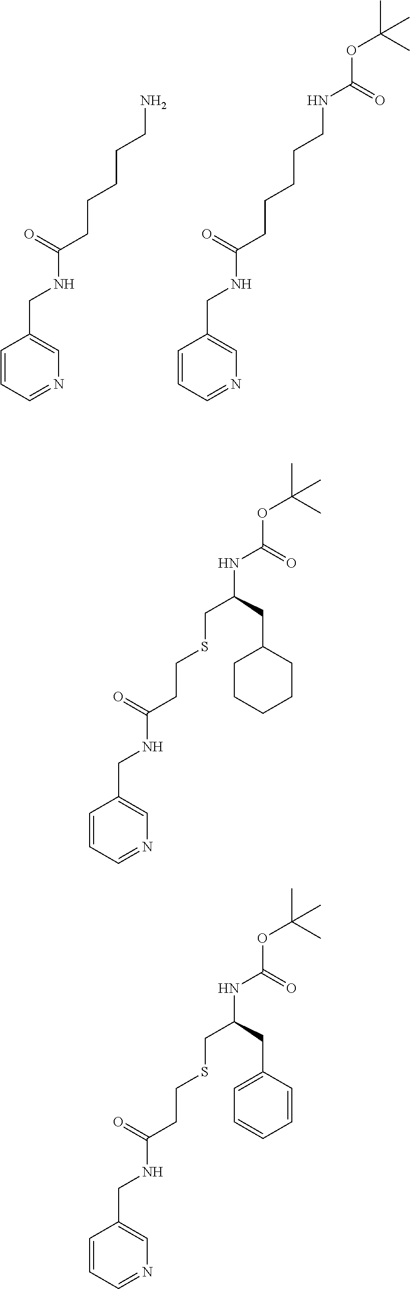 [SMILES: NCCCCCC(=O)NCc1cccnc1 ; CC(C)(C)OC(=O)NCCCCCC(=O)NCc1cccnc1 ; CC(C)(C)OC(=O)N[C@H](CSCCC(=O)NCc1cccnc1)CC1CCCCC1 ; CC(C)(C)OC(=O)N[C@H](CSCCC(=O)NCc1cccnc1)Cc1ccccc1]

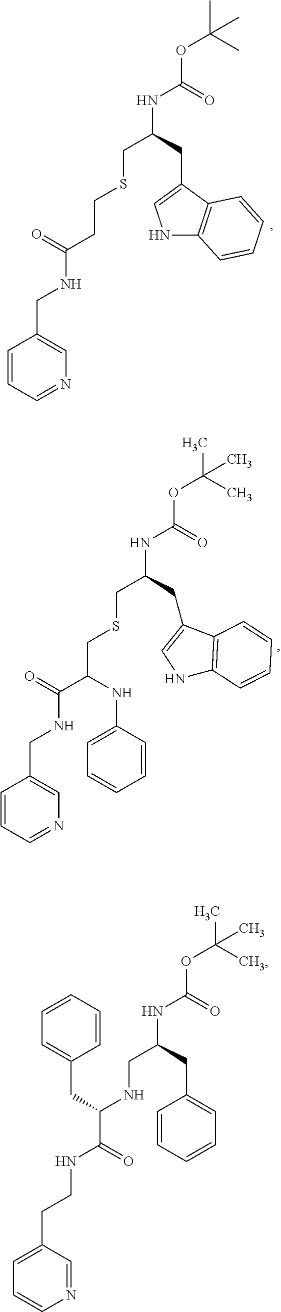 [SMILES: CC(C)(C)OC(=O)N[C@H](CSCCC(=O)NCc1cccnc1)Cc1c[nH]c2ccccc12 ; CC(C)(C)OC(=O)N[C@H](CSCC(Nc1ccccc1)C(=O)NCc1cccnc1)Cc1c[nH]c2ccccc12 ; CC(C)(C)OC(=O)N[C@H](CN[C@@H](Cc1ccccc1)C(=O)NCCc1cccnc1)Cc1ccccc1]

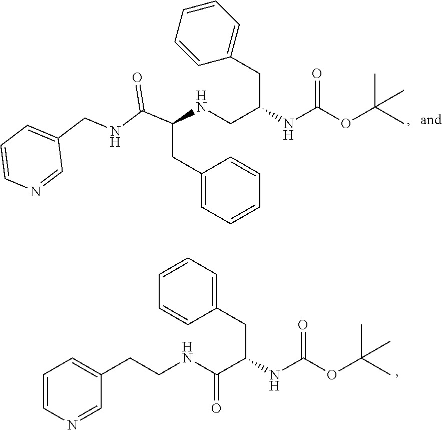 [SMILES: CC(C)(C)OC(=O)N[C@H](CN[C@@H](Cc1ccccc1)C(=O)NCc1cccnc1)Cc1ccccc1 ; CC(C)(C)OC(=O)N[C@@H](Cc1ccccc1)C(=O)NCCc1cccnc1]

a salt or solvate thereof, and any combinations thereof.

In one embodiment, the compound is selected from the group consisting of:

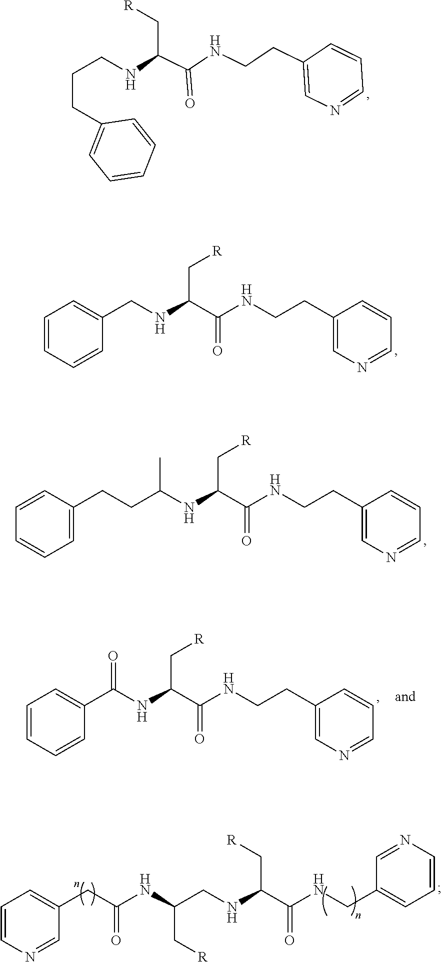

wherein each occurrence of R is selected from the group consisting of phenyl and indole; and

wherein each occurrence of n is selected from the group consisting of 1 and 2.

In one embodiment, the invention includes a composition comprising a compound of Formula (I). In another embodiment, the composition further comprises a pharmaceutically acceptable carrier.

The invention also includes a method of CYP3A4 in a subject in need thereof The method includes the step of administering to the subject a therapeutically effective amount of a composition comprising at least one compound of Formula (I):

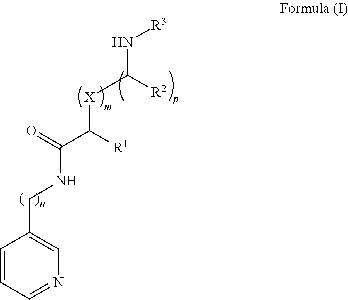

wherein in Formula (I):

R¹ and R² are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, substitued -(C₁-C₆)alkyl-heteroaryl, and N(R⁴)(R⁵);

R³ is selected from the group consisting of H, -C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substituted -(C₁-C₆)alkyl-heteroaryl and —C(═O)R⁶, wherein the alkyl group is optionally substituted;

R⁴ and R⁵ are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substitued-(C₁-C₆)alkyl-heteroaryl;

R⁶ is selected from the group consisting of phenyl, substituted phenyl, -(C₁-C₆)alkyl-phenyl, substitued -(C₁-C₆) alkyl-phenyl, -(C₁-C₆)alkyl-heteroaryl, substitued -(C₁-C₆)alkyl-heteroarly, and OR⁷;

R⁷ is selected from the group consisting of H, -C₁-C₆ alkyl, substituted C₁-C₆ alkyl, aryl, cycloalkyl phenyl, substituted phenyl, heteroaryl, substituted heteroaryl, heterocyclyl, substituted heterocyclyl, -(C₁-C₆)alkyl-phenyl, substituted -(C₁-C₆)alkyl-aryl, substituted -(C₁-C₆)alkyl-aryl, -(C₁-C₆)alkyl-carbocyclic, -(C₁-C₆)alkyl-heteroaryl, substitued -(C₁-C₆)alkyl- heteroaryl;

each occurrence of X is independently selected from the group consisting of CH₂, NH, S, and O;

m is an integer from 0 to 3;

n is an integer from 0 to 3; and

p is an integer from 0 to 1;

a salt or solvate, and any combinations thereof

In one embodiment, R¹ is selected from the group consisting of H, benzyl, and aniline.

In one embodiment, R² is selected from the group consisting of H, -(C₁-C₆)alkyl-carbocyclic, -(C₁-C₆)alkyl-phenyl, -(C₁-C₆)alkyl-substituted phenyl, -(C₁-C₆)alkyl-heteroaryl, and -(C₁-C₆)alkyl-substituted heteroaryl.

In one embodiment, R⁷ is -C₁-C₆ alkyl.

In one embodiment, m is 1. In another embodiment, n is 1.

In one embodiment, X is S.

In one embodiment, the compound is selected from the group consisting of:

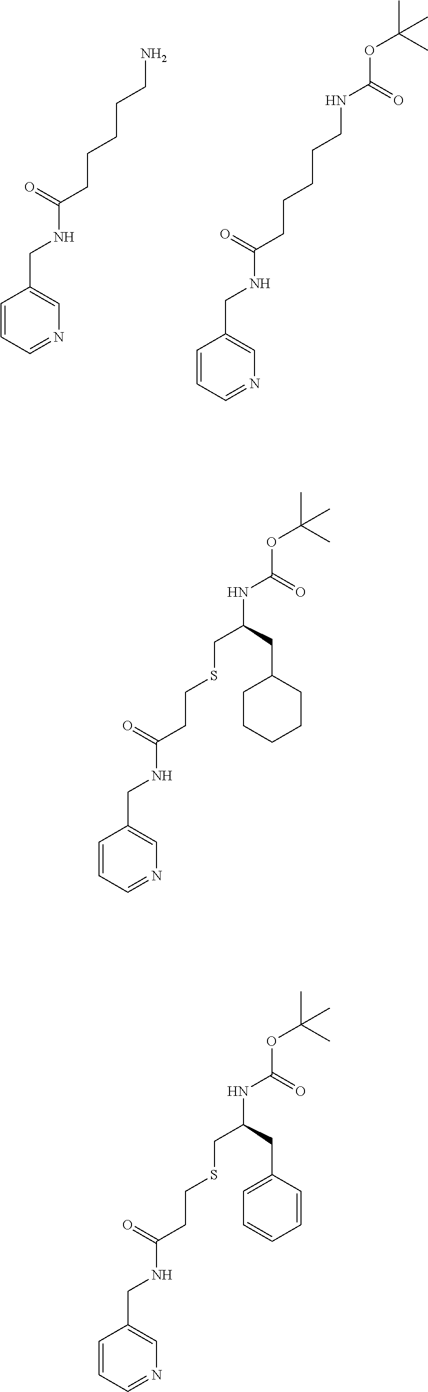 [SMILES: NCCCCCC(=O)NCc1cccnc1 ; CC(C)(C)OC(=O)NCCCCCC(=O)NCc1cccnc1 ; CC(C)(C)OC(=O)N[C@H](CSCCC(=O)NCc1cccnc1)CC1CCCCC1 ; CC(C)(C)OC(=O)N[C@H](CSCCC(=O)NCc1cccnc1)Cc1ccccc1]

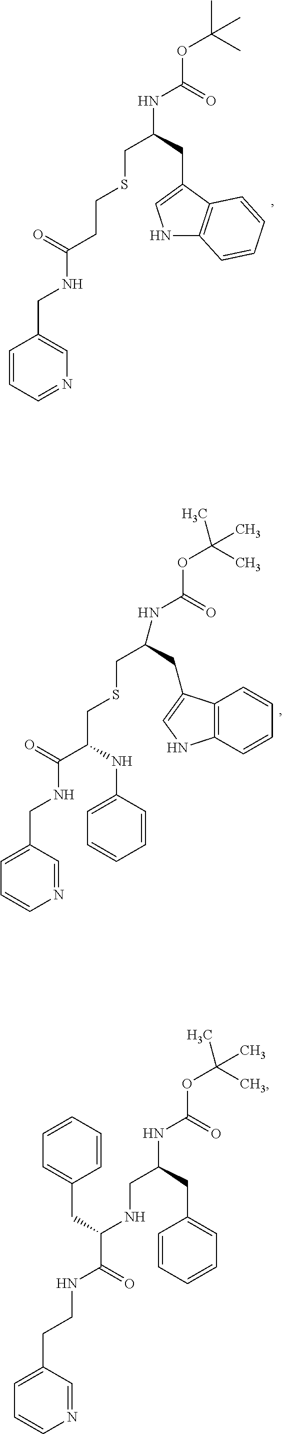 [SMILES: CC(C)(C)OC(=O)N[C@H](CSCCC(=O)NCc1cccnc1)Cc1c[nH]c2ccccc12 ; CC(C)(C)OC(=O)N[C@H](CSC[C@H](Nc1ccccc1)C(=O)NCc1cccnc1)Cc1c[nH]c2ccccc12 ; CC(C)(C)OC(=O)N[C@H](CN[C@@H](Cc1ccccc1)C(=O)NCCc1cccnc1)Cc1ccccc1]

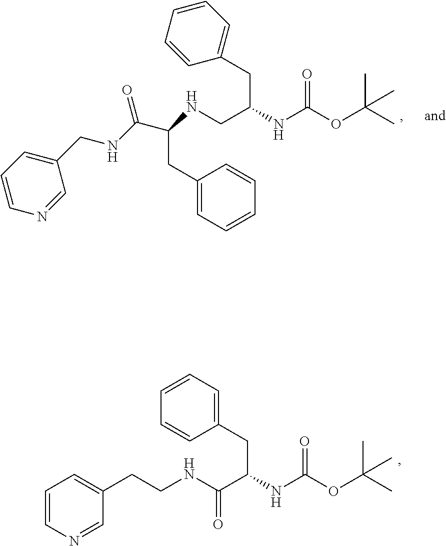 [SMILES: CC(C)(C)OC(=O)N[C@H](CN[C@@H](Cc1ccccc1)C(=O)NCc1cccnc1)Cc1ccccc1 ; CC(C)(C)OC(=O)N[C@@H](Cc1ccccc1)C(=O)NCCc1cccnc1]

a salt or solvate thereof, and any combinations thereof.

In one embodiment, the compound is selected from the group consisting of:

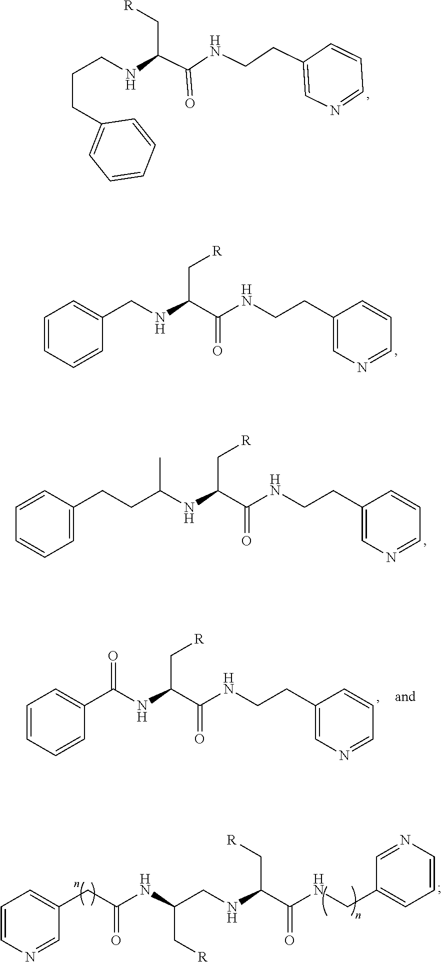

wherein each occurrence of R is selected from the group consisting of phenyl and indole; and

wherein each occurrence of n is independently 1 or 2.

In one embodiment, the method further includes administering to the subject at least one additional therapeutic agent. In another embodiment, the therapeutic agent is an antiviral agent. In yet another embodiment the therapeutic agent is a protease inhibitor. In another embodiment, the composition and the additional therapeutic agent are co-administered. In another embodiment the composition and the additional therapeutic agent are co-formulated.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts the chemical structures of ritonavir, cobicistat and GS3. The phenyl side groups proximal and distant from the heme-ligating moiety are designated as Phe-1 and Phe-2, respectively.

FIG. 2 depicts the pharmacophore model for a CYP3A4-specific inhibitor derived from the structure/function studies on analogues of ritonavir. Pharmacophoric determinants: I—strong heme-ligating nitrogen donor, II—flexible backbone, III and IV—aromatic and hydrophobic moieties, respectively, V—hydrogen donor/acceptor, IV—poly-functional end-group. The P1 and P2 sites are hydrophobic pockets adjacent to the phenylalanine cluster and the heme, respectively.

FIG. 3 depicts a scaffold used for the CYP3A4 inhibitor design. The pyridine ring, serving as a heme-ligating moiety, is attached to a flexible backbone via a peptide bond, an H-donor/acceptor. By varying the side (R¹ and R²) and terminal (R³) groups, the role of each moiety in the CYP3A4 binding and inhibition can be probed.

FIG. 4 depicts the chemical structures of the investigated compounds.

FIG. 5, comprising FIGS. 5A-5C, depicts the spectral changes induced in CYP3A4. Absorbance spectra of the ferric ligand-free, ligand-bound and ferrous ligand-bound forms are shown in solid, dashed and dotted lines, respectively. FIG. 5A depicts the spectral changes induced in CYP3A4 by pyridine. FIG. 5B depicts the spectral changes induced in CYP3A4 by aminoethylpyridine. FIG. 5C depicts the spectral changes induced in CYP3A4 by compound 5. Insets (a) are absorbance changes observed during equilibrium titration of CYP3A4 with pyridine, aminoethylpyridine and 5. Insets (b) are plots of absorbance change vs. ligand concentration.

FIG. 6, comprising FIGS. 6A-6D, depicts spectral changes induced in CYP3A4. FIG. 6A depicts the spectral change induced in CYP3A4 by compound 4. FIG. 6b depicts the spectral change induced in CYP3A4 by compound 11. FIG. 6C depicts the spectral change induced in CYP3A4 by compound 15a. FIG. 6D depicts the spectral change induced in CYP3A4 by compound 15b. Absorbance spectra of the ferric ligand-free, ligand-bound and ferrous ligand-bound forms are shown in solid, dashed and dotted lines, respectively. Absorbance maxima of the ferric and ferrous ligand-bound CYP3A4 are indicated. Insets (a) are absorbance changes observed during equilibrium titration of CYP3A4 with 4, 11 and 15a-b. Insets (b) are plots of absorbance change vs. ligand concentration.

FIG. 7, comprising FIGS. 7A-7D, depicts the crystal structure active site of CYP3A4 bound to compounds. FIG. 7A depicts the active site of CYP3A4 bound to 15a. FIG. 7A depicts the active site of CYP3A4 bound to 15b. The ligands (in green), heme (in pink) and the Arg105 side group are shown in stick representation. 2Fo-Fc electron density maps are contoured at 16. FIG. 7C shows that a 180° rotation of 15a (green) and 15b (light gray) relative to GS3 (magenta) prevents H-bond formation with Ser119. FIG. 7d demonstrates that the Boc groups of 15a and 15b occupy the P1 site and displace the I-helix Phe304 to the same extent as GS3 Phe-1 does.

FIG. 8, comprising FIGS. 8A-8C, depicts the 1.93 Å crystal structure of CYP3A4 K282A/E285A bound to 4 and imidazole. FIG. 8A is an image of imidazole (in magenta) ligating to the heme, whereas compound 4 binds nearby. The complex is stabilized by two hydrogen bonds (depicted as red dotted lines) established between the carbonyl oxygen and amide nitrogen of 4 and the imidazole nitrogen and Ser119 hydroxyl group, respectively. 2Fo-Fc electron density map is contoured at 1σ. FIG. 8B depicts another view at the active site showing how the pyridine ring of 4 is positioned relative to the phenylalanine cluster (residues 108, 213, 215, 241 and 304). FIG. 8C is an image of superposition of the 4- and GS3-bound structures (in green and magenta, respectively). H-bonds to Ser119 are shown as red dotted lines.

FIG. 9, comprising FIGS. 9A-9B, depicts the 2.25 Å crystal structure of CYP3A4 K282A/E285A ligated to 4. FIG. 9A demonstrates compound 4 directly binds to the heme via the pyridine nitrogen. The 4 backbone bends in order to place the end portion into the P2 site. 2Fo-Fc electron density map is contoured at 1σ. FIG. 9B depicts the superposition of the 4- and GS3-bound structures and reveals that, similar to 15a and 15b, compound 4 rotates around the pyridine nitrogen by ˜180° to optimize hydrophobic interactions at the P2 site through the aliphatic backbone and Boc moiety.

FIG. 10 depicts an exemplary synthesis of compounds 4 and 5.

FIG. 11 depicts an exemplary synthesis of first generation analog compound 11.

FIG. 12 depicts an exemplary synthesis of first generation analogs compounds 15a and 15b.

FIG. 13 depicts the data collection and refinement statistics of 4 +imidazole, 4, 15a, and 15b.

FIG. 14 is a ¹H NMR spectrum of Compound 5 in CD₃OD.

FIG. 15 is a ¹³C NMR spectrum of Compound 5 in CD₃OD.

FIG. 16 is a mass spectrum of Compound 5.

FIG. 17 is a ¹H NMR spectrum of Compound 4 in CDCl₃.

FIG. 18 is a ¹³C NMR spectrum of Compound 4 in CDCl₃.

FIG. 19 is a mass spectrum of Compound 4.

FIG. 20 is a ¹H NMR spectrum of Compound 12a.

FIG. 21 is a ¹H NMR spectrum of Compound 13a.

FIG. 22 is a mass spectrum of Compound 14a.

FIG. 23 is a ¹H NMR spectrum of Compound 15a in CDCl₃.

FIG. 24 is a ¹³C NMR spectrum of Compound 15a in CDCl₃.

FIG. 25 is a mass spectrum of Compound 15a.

FIG. 26 is a mass spectrum of Compound 15a.

FIG. 27 is a mass spectrum of Compound 13b.

FIG. 28 is a ¹H NMR spectrum of Compound 14b.

FIG. 29 is a ¹H NMR spectrum of Compound 15b in CDCl₃.

FIG. 30 is a ¹³C NMR spectrum of Compound 15b in CDCl₃.

FIG. 31 is a mass spectrum of Compound 15b.

FIG. 32 is a mass spectrum of Compound 10.

FIG. 33 is a mass spectrum of Compound 11.

FIG. 34 is a ¹H NMR spectrum of Compound 11 in CD₂Cl₂.

FIG. 35 is a ¹³C NMR spectrum of Compound 11 in CD₂Cl₂.

FIG. 36 depicts the chemical structures of compounds 01ERS083 and 01ERS089.

FIG. 37 depicts results from experiments showing the spectral changes induced by 01ERS083 and 01ERS089 in CYP3A4. Left insets depict difference spectra between the ligand-free and inhibitor-bound forms. Right insets are plots of the absorbance change versus inhibitor concentration, based on which the K_{d} values were calculated.

FIG. 38 depicts an exemplary synthesis of compound 01ERS083.

FIG. 39 depicts an exemplary synthesis of compound 01ERS089.

FIG. 40 depicts the chemical structures of compounds 01ERS087 and 01ERS104.

FIG. 41 depicts alternative synthetic routes for for the synthesis of compounds of the invention.

FIG. 42 depicts the chemical structures of useful CYP3A4 inhibitors.

### DETAILED DESCRIPTION OF THE INVENTION

This invention includes the unexpected identification of novel compounds that are useful for inhibition of cytochrome P450 3A4 (CYP3A4). As demonstrated herein, the compounds of the present invention have been shown to be effective inhibitors of CYP3A4.

Human cytochrome P450 3A4 (CYP3A4) is a key xenobiotic-metabolizing enzyme that oxidizes and clears the majority of drugs. CYP3A4 inhibition may be beneficial and enhance therapeutic efficiency of co-administered pharmaceuticals that are metabolized by CYP3A4. Based on investigations of analogs of ritonavir, a potent CYP3A4 inactivator and pharmacoenhancer, a pharmacophore model has been built for a CYP3A4-specific inhibitor. The data presented herein is the first attempt to test this model using a set of rationally designed compounds. The functional and structural data presented herein agree well with the proposed pharmacophore. In particular, the importance of a flexible backbone, the H-bond donor/acceptor moiety and aromaticity of the side group analogous to Phe-2 of ritonavir were confirmed, and the leading role of hydrophobic interactions at the sites adjacent to the heme and phenylalanine cluster in the ligand binding process was demonstrated. The X-ray structures of CYP3A4 bound to the rationally designed inhibitors provide deeper insights into the mechanism of the CYP3A4-ligand interaction. Most importantly, compounds of the present invention are less complex than ritonavir, have comparable sub-micromolar affinity and inhibitory potency for CYP3A4 and, thus, could serve as templates for synthesis of second generation inhibitors for further evaluation and optimization of the pharmacophore model.

The present invention also includes novel methods of inhibiting CYP3A4 in a patient in need thereof using the compounds of the invention.

The present invention also includes a composition comprising at least one compound of the invention, wherein the composition optionally further comprises at least one additional therapeutic agent. In one embodiment, the additional therapeutic agent is an antiviral agent.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a sign or symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a nontoxic but sufficient amount of an agent to provide the desired biological result. That result may be reduction and/or alleviation of a sign, a symptom, or a cause of a disease or disorder, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

An “effective amount” of a delivery vehicle is that amount sufficient to effectively bind or deliver a compound.

As used herein, the term “potency” refers to the dose needed to produce half the maximal response (ED₅₀).

As used herein, the term “efficacy” refers to the maximal effect (Eₘₐₓ) achieved within an assay.

As used herein, the term “alkyl,” by itself or as part of another substituent means, unless otherwise stated, a straight or branched chain hydrocarbon having the number of carbon atoms designated (i.e. C₁⁻₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C≡N, —(═O)O(C₁-C₄)alkyl, 13 C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

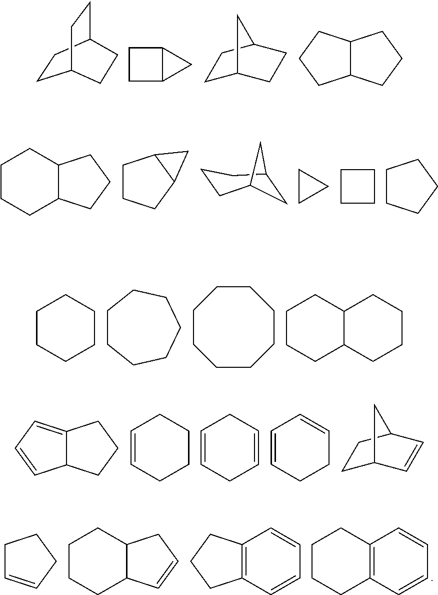

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

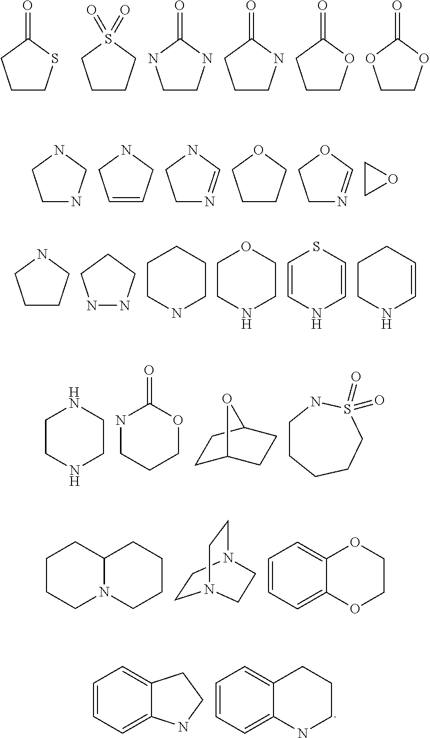

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂- and aryl-CH(CH₃)-. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

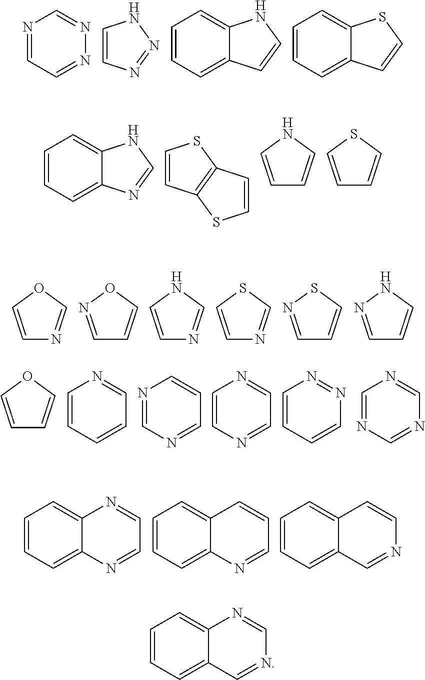

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds Useful Within the Invention

The compounds of the present invention may be synthesized using techniques well-known in the art of organic synthesis. The starting materials and intermediates required for the synthesis may be obtained from commercial sources or synthesized according to methods known to those skilled in the art.

In one aspect, the compound of the invention is a compound of Formula (I), or a salt thereof:

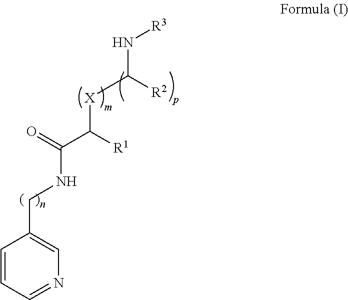

wherein in Formula (I):

R¹ and R² are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, substitued -(C₁-C₆)alkyl-heteroaryl, and N(R⁴)(R⁵);

R³ is selected from the group consisting of H, -C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substituted -(C₁-C₆)alkyl-heteroaryl and —C(═O)R⁶, wherein the alkyl group is optionally substituted;

R⁴ and R⁵ are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substitued-(C₁-C₆)alkyl-heteroaryl;

R⁶ is selected from the group consisting of phenyl, substituted phenyl, -(C₁-C₆)alkyl-phenyl, substitued -(C₁-C₆) alkyl-phenyl, -(C₁-C₆)alkyl-heteroaryl, substitued -(C₁-C₆)alkyl-heteroarly, and OR⁷;

R⁷ is selected from the group consisting of H, -C₁-C₆ alkyl, substituted C₁-C₆ alkyl, aryl, cycloalkyl phenyl, substituted phenyl, heteroaryl, substituted heteroaryl, heterocyclyl, substituted heterocyclyl, -(C₁-C₆)alkyl-phenyl, substituted -(C₁-C₆)alkyl-aryl, substituted -(C₁-C₆)alkyl-aryl, -(C₁-C₆)alkyl-carbocyclic, -(C₁-C₆)alkyl-heteroaryl, substitued -(C₁-C₆)alkyl- heteroaryl;

each occurrence of X is independently selected from the group consisting of CH₂, NH, S, and O;

m is an integer from 0 to 3;

n is an integer from 0 to 3; and

p is an integer from 0 to 1;

a salt or solvate, and any combinations thereof.

In one embodiment, R¹ is selected from the group consisting of H, benzyl, and aniline.

In one embodiment, R² is selected from the group consisting of H, -(C₁-C₆)alkyl-carbocyclic, -(C₁-C₆)alkyl-phenyl, -(C₁-C₆)alkyl-substituted phenyl, -(C₁-C₆)alkyl-heteroaryl, and -(C₁-C₆)alkyl-substituted heteroaryl.

In one embodiment, R⁷ is -C₁-C₆ alkyl.

In one embodiment, m is 1. In another embodiment, n is 1.

In one embodiment, X is S. In one embodiment X is CH₂

In one embodiment, the compound is selected from the group consisting of:

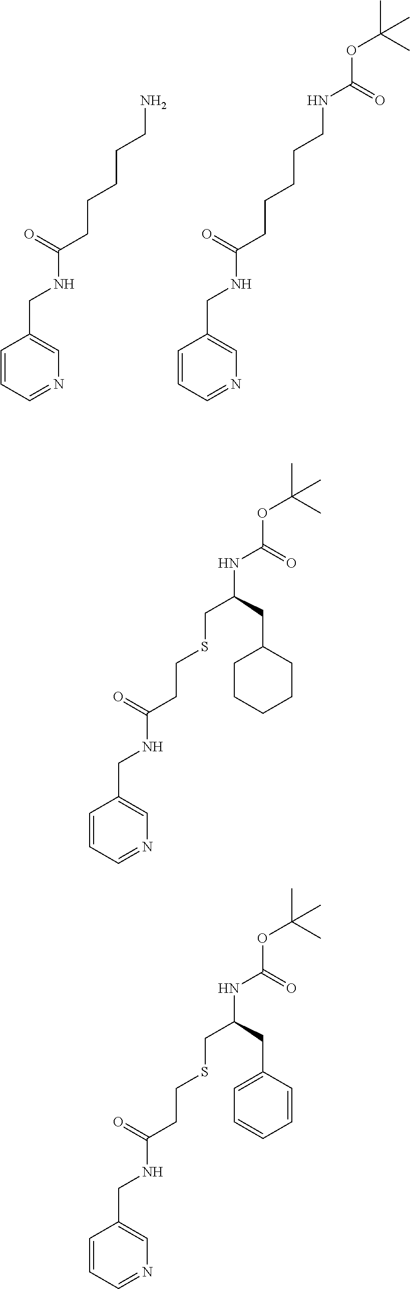 [SMILES: NCCCCCC(=O)NCc1cccnc1 ; CC(C)(C)OC(=O)NCCCCCC(=O)NCc1cccnc1 ; CC(C)(C)OC(=O)N[C@H](CSCCC(=O)NCc1cccnc1)CC1CCCCC1 ; CC(C)(C)OC(=O)N[C@H](CSCCC(=O)NCc1cccnc1)Cc1ccccc1]

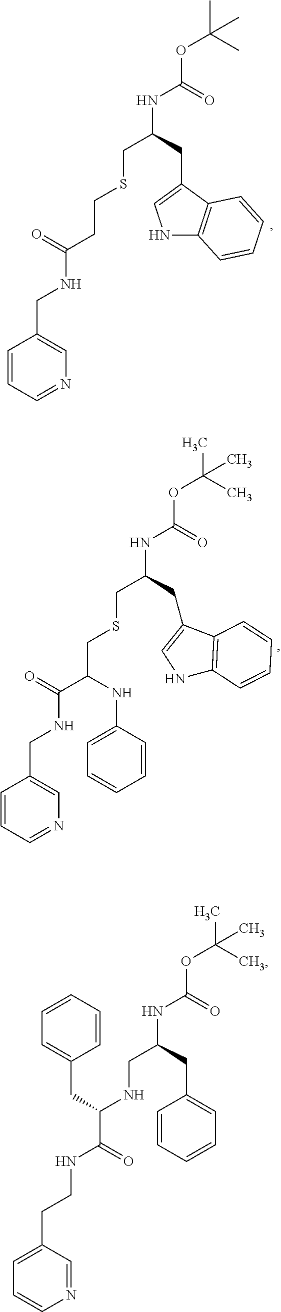 [SMILES: CC(C)(C)OC(=O)N[C@H](CSCCC(=O)NCc1cccnc1)Cc1c[nH]c2ccccc12 ; CC(C)(C)OC(=O)N[C@H](CSCC(Nc1ccccc1)C(=O)NCc1cccnc1)Cc1c[nH]c2ccccc12 ; CC(C)(C)OC(=O)N[C@H](CN[C@@H](Cc1ccccc1)C(=O)NCCc1cccnc1)Cc1ccccc1]

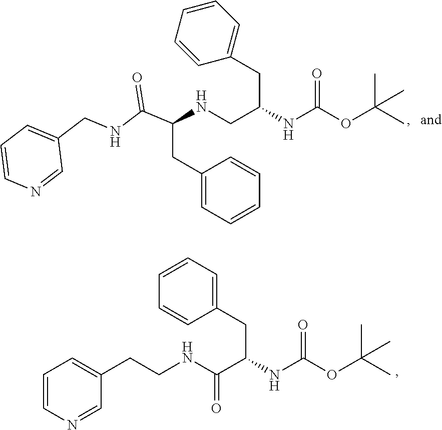 [SMILES: CC(C)(C)OC(=O)N[C@H](CN[C@@H](Cc1ccccc1)C(=O)NCc1cccnc1)Cc1ccccc1 ; CC(C)(C)OC(=O)N[C@@H](Cc1ccccc1)C(=O)NCCc1cccnc1]

a salt or solvate thereof, and any combinations thereof.

In one embodiment, the compound is selected from the group consisting of:

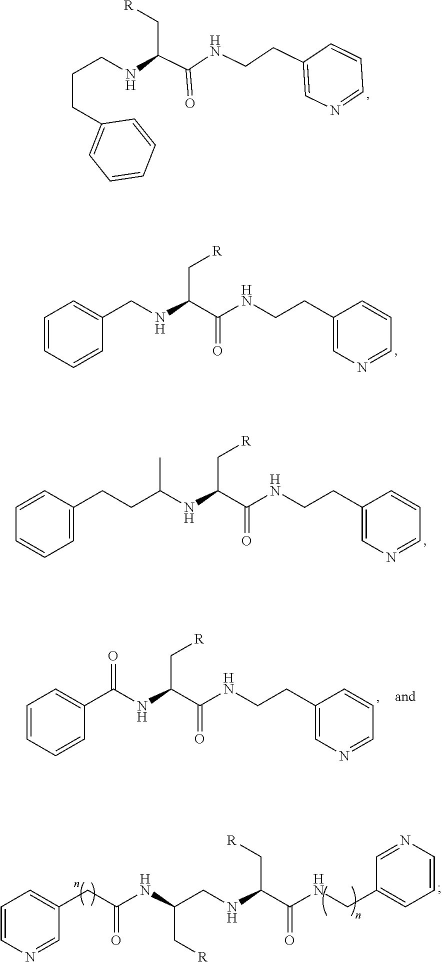

wherein each occurrence of R is selected from the group consisting of phenyl and indole; and

wherein each occurrence of n is independently 1 or 2.

### Preparation of the Compounds of the Invention

Compounds of Formula (I) may be prepared by the general schemes described herein, using the synthetic method known by those skilled in the art. The following examples illustrate non-limiting embodiments of the invention.

In a non-limiting embodiment, the primary alcohol of compound A is converted to a leaving group, such as a tosyl group. For example, compound A is treated with p-toluenesulfonyl chloride and a base such as triethylamine to form tosylate B, in which tosyl group is then displaced by thiol C in the presence of a base to yield carboxylic acid D. D is then treated with an amine E, 1-ethyl-3-(3-dimethylaminopropyl)carbodiimide (EDAC) and a base such as diisoproyplyethylamine (DIPEA) to produce amide F.

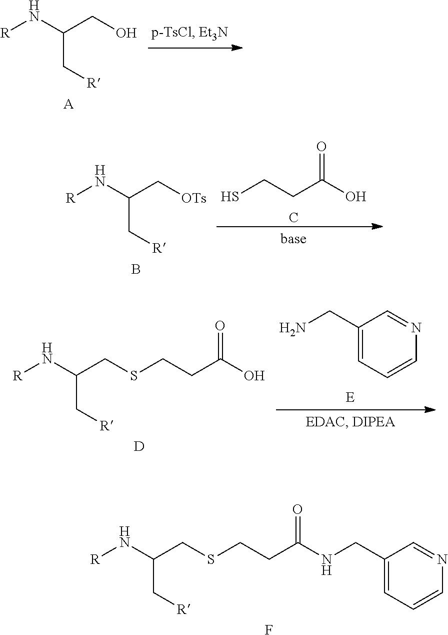

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶C, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸_{F,} ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John

Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference in their entirety). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

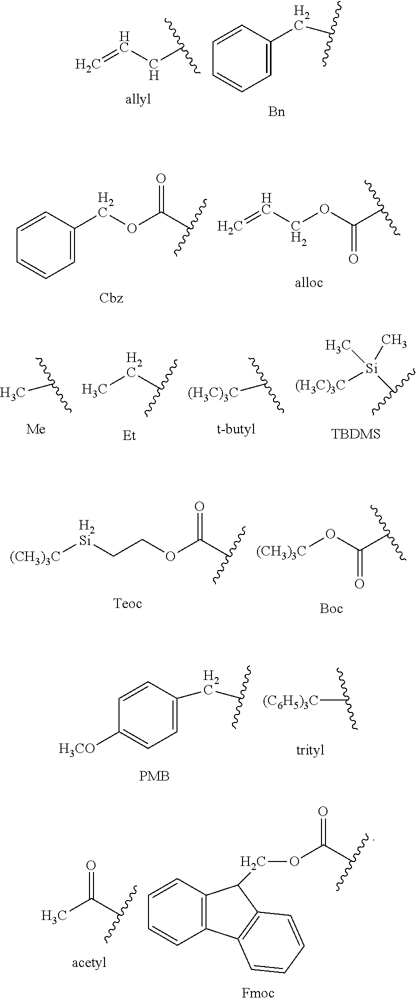

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Methods of the Invention

The invention includes a method of inhibiting CYP3A4 in a subject in need thereof. The method comprises administering to the subject an effective amount of a therapeutic composition comprising a compound of the invention. The method further comprises administering to the subject an effective amount of a therapeutic composition comprising a compound of the invention for the treatment of HIV.

In one embodiment, the method further comprises administering to the subject an additional therapeutic agent.

In one embodiment, the compound of the invention and the therapeutic agent are co-administered to the subject. In another embodiment, the compound of the invention and the therapeutic agent are coformulated and co-administered to the subject.

In one embodiment the therapeutic agent is an antiviral agent.

In one embodiment, administering the compound of the invention to the subject allows for administering a lower dose of the therapeutic agent compared to the dose of the therapeutic agent alone that is required to achieve similar results in treating the subject. For example, in one embodiment, the compound of the invention inhibits metabolism of the additional therapeutic compound, thereby allowing for a lower dose of the therapeutic compound to provide the same effect. In another embodiment, the compound of the invention inhibits metabolism of the HIV therapeutic compounds and thereby act as a pharmacoenhancer.

In one embodiment, the subject is a mammal. In another embodiment, the mammal is a human.

### Combination Therapies

The compounds of the present invention may be useful in combination with one or more additional compounds. In certain embodiments, these additional compounds may comprise compounds of the present invention or therapeutic agents which are known antivirals. In certain embodiments, the antiviral agent may comprise compounds useful for treating HIV infections. Such compounds include, but are not limited to, compounds which are known to treat, prevent, or reduce the symptoms of HIV infections

In non-limiting examples, the compounds useful within the invention may be used in combination with one or more of the following anti-HIV drugs:

HIV Combination Drugs: efavirenz, emtricitabine or tenofovir disoproxil fumarate (Atripla®/BMS, Gilead); lamivudine or zidovudine (Combivir®/GSK); abacavir or lamivudine (Epzicom®/GSK); abacavir, lamivudine or zidovudine (Trizivir®/GSK); emtricitabine, tenofovir disoproxil fumarate (Truvada®/Gilead).

Entry and Fusion Inhibitors: maraviroc (Celsentri®, Selzentry®/Pfizer); pentafuside or enfuvirtide (Fuzeon®/Roche, Trimeris).

Integrase Inhibitors: raltegravir or MK-0518 (Isentress®/Merck).

Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).

Nucleoside Reverse Transcriptase Inhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddI, dideoxyinosine or didanosine (Videx®/BMS); abacavir sulfate plus lamivudine (Epzicom®/GSK); stavudine, d4T, or estavudina (Zerit®/BMS); tenofovir, PMPA prodrug, or tenofovir disoproxil fumarate (Viread®/Gilead).

Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec); fosamprenavir (Telzir®, Lexiva®/GSK, Vertex); indinavir sulfate (Crixivan®/Merck); saquinavir mesylate (Invirase®/Roche); lopinavir or ritonavir (Kaletra®/Abbott); nelfinavir mesylate (Viracept®/Pfizer); ritonavir (Norvir®/Abbott).

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either before or after the onset of a disease or infection. Further, several divided dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, (e.g., human), may be carried out using known procedures, at dosages and for periods of time effective to treat the disease or infection in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or infection in the patient. Dosage regimens may be adjusted to provide the optimum therapeutic response. For example, several divided doses may be administered daily. In another example, the dose may be proportionally reduced as indicated by the exigencies of the therapeutic situation. A non-limiting example of an effective dose range for a therapeutic compound of the invention is from about 1 mg/kg to about 5,000 mg/kg of body weight/per day. One of ordinary skill in the art would be able to assess the relevant factors and make the determination regarding the effective amount of the therapeutic compound without undue experimentation.

Actual dosage levels of the active ingredients in the pharmaceutical compositions of this invention may be varied to obtain an amount of the active ingredient that is effective to achieve the desired therapeutic response for a particular patient, composition, and mode of administration, without generating excessive side effects in the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical professional, e.g., physician or veterinarian, having ordinary skill in the art may readily determine and prescribe the effective amount of the pharmaceutical composition required. For example, the physician or veterinarian could start with a dosage of the compound of the invention in the pharmaceutical composition at a level that is lower than the level required to achieve the desired therapeutic effect, and then increase the dosage over time until the desired effect is achieved.

In particular embodiments, it is advantageous to formulate the compound in dosage unit form for ease of administration and uniformity of dosage. “Dosage unit form” as used herein refers to a physically discrete unit containing a predetermined quantity of therapeutic compound calculated to produce the desired therapeutic effect, in association with the required pharmaceutical vehicle. The dosage unit forms of the invention can be selected based upon (a) the unique characteristics of the therapeutic compound and the particular therapeutic effect to be achieved, and (b) the limitations inherent in the art of compounding/formulating such a therapeutic compound for the treatment of a disease or infection in a patient.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), vegetable oils, and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is useful to include isotonic agents, for example, sugars, sodium chloride, or polyalcohols such as mannitol and sorbitol, in the composition. Prolonged absorption of the injectable compositions can be achieved by including in the composition an agent which delays absorption, for example, aluminum monostearate or gelatin. In one embodiment, the pharmaceutically acceptable carrier is DMSO, alone or in combination with other carriers.

The therapeutically effective amount or dose of a compound of the present invention depends on the age, sex and weight of the patient, the current medical condition of the patient and the severity of the disease or infection in the patient being treated. The skilled artisan is able to determine appropriate doses depending on these and other factors.

The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Doses of the compound of the invention for administration may be in the range of from about 1 μg to about 10,000 mg, from about 20 μg to about 9,500 mg, from about 40 μg to about 9,000 mg, from about 75 μg to about 8,500 mg, from about 150 μg to about 7,500 mg, from about 200 μg to about 7,000 mg, from about 3050 μg to about 6,000 mg, from about 500 μg to about 5,000 mg, from about 750 μg to about 4,000 mg, from about 1 mg to about 3,000 mg, from about 10 mg to about 2,500 mg, from about 20 mg to about 2,000 mg, from about 25 mg to about 1,500 mg, from about 30 mg to about 1,000 mg, from about 40 mg to about 900 mg, from about 50 mg to about 800 mg, from about 60 mg to about 750 mg, from about 70 mg to about 600 mg, from about 80 mg to about 500 mg, and any and all whole or partial increments therebetween.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, the dosage of a second compound as described elsewhere herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are administered to the patient from about one to about five times per day or more. In various embodiments, the compositions of the invention are administered to the patient, 1-7 times per day, 1-7 times every two days, 1-7 times every 3 days, 1-7 times every week, 1-7 times every two weeks, and 1-7 times per month. It is readily apparent to one skilled in the art that the frequency of administration of the various combination compositions of the invention will vary from individual to individual depending on many factors including, but not limited to, age, the disease or disorder to be treated, the severity of the disease or disorder to be treated, gender, overall health, and other factors. Thus, the invention should not be construed to be limited to any particular dosing regime and the precise dosage and composition to be administered to any patient is determined by the medical professional taking all other factors about the patient into account.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, may be reduced to a level at which the improved disease is retained. In some embodiments, a patient may require intermittent treatment on a long-term basis, or upon any recurrence of the disease or disorder.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the present invention is directed to a packaged pharmaceutical composition comprising a container holding a therapeutically effective amount of a compound of the invention, alone or in combination with a second pharmaceutical agent; and instructions for using the compound to treat or prevent a disease or infection in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, suitable forms include tablets, dragees, liquids, drops, suppositories, or capsules, caplets and gelcaps. The compositions formulated for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation involves the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion.

Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release refers to a drug formulation that provides for gradual release of a drug over an extended period of time, and that may, although not necessarily, result in substantially constant blood levels of a drug over an extended time period. The period of time may be as long as a day, a week, or a month or more and should be a release which is longer that the same amount of agent administered in bolus form. The term delayed release is used herein in its conventional sense to refer to a drug formulation that provides for an initial release of the drug after some delay following drug administration and that mat, although not necessarily, includes a delay of from about 10 minutes up to about 12 hours.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term pulsatile release refers to a drug formulation that provides release of the drug in such a way as to produce pulsed plasma profiles of the drug after drug administration.

The term immediate release refers to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art recognize, or are able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Evaluation of the Pharmacophore Model for a CYP3A4-Specific Inhibitor via Rational Inhibitor Design

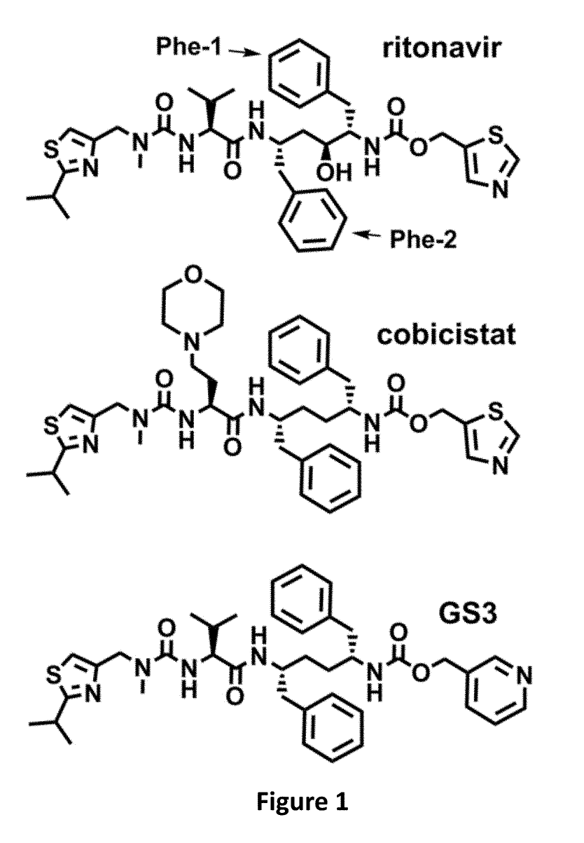

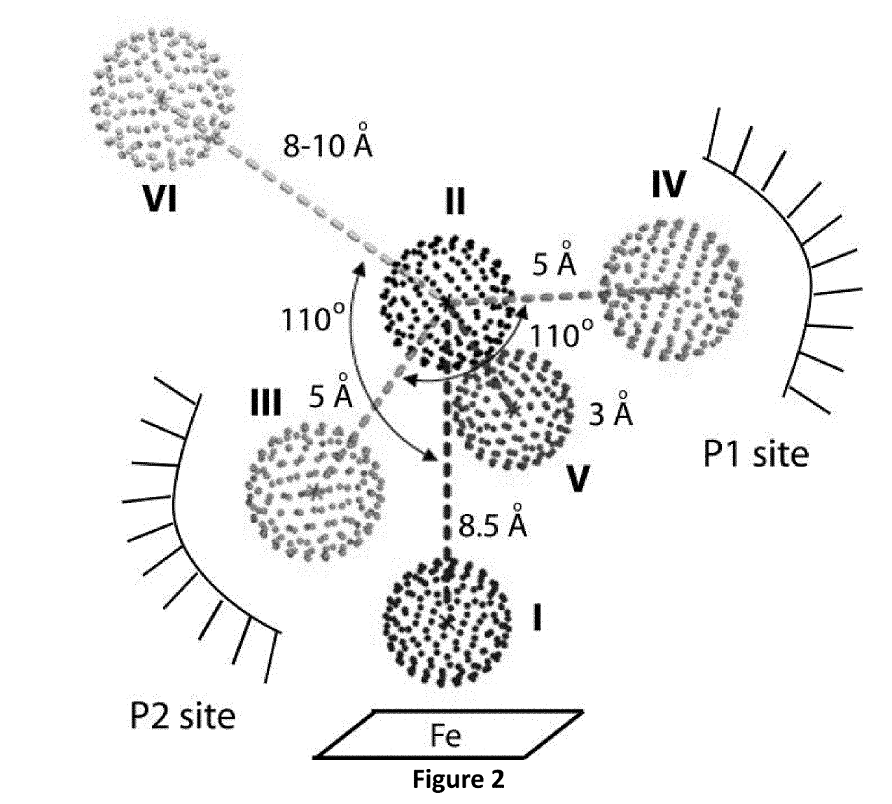

The accumulated functional and structural data enabled the identification of several important trends in the ligand binding process (Sevrioukova and Poulos, 2014, Curr Top Med Chem 14:1348-55). In particular: (i) strong binding through the heme-ligating moiety is a prerequisite for potent CYP3A4 inhibition, and pyridine possesses more favorable stereoelectronic properties for the heme ligation than any other chemical groups tested; (ii) the flexible backbone enables a better fit into the CYP3A4 active site; (iii) the side group at a Phe-2 position (indicated in FIG. 1) is critically important because it not only fills the hydrophobic cavity (P2 site) but also has a potential to stabilize the protein-ligand complex via different types of interactions with the heme-ligating group and nearby Arg105; (iv) occupation of the pocket hosting the Phe-1 group (P1 site) is also important and greatly improves inhibitory potency; (v) polar interactions with Ser119 increase the ligand affinity and association rate, regardless of whether the hydrogen bond with the ligand is established or not; and (vi) the terminal group modulates the CYP3A4-inhibitor complex affinity and stability which, in turn, correlate with the ligand-induced changes in the melting temperature and the amplitude of the 442 nm absorption of the ferrous ligand-bound form. Having mapped the inhibitory determinants within the CYP3A4 active site, a pharmacophore model was built for a CYP3A4-specific inhibitor (FIG. 2) (Sevrioukova and Poulos, 2014, Curr Top Med Chem 14:1348-55).

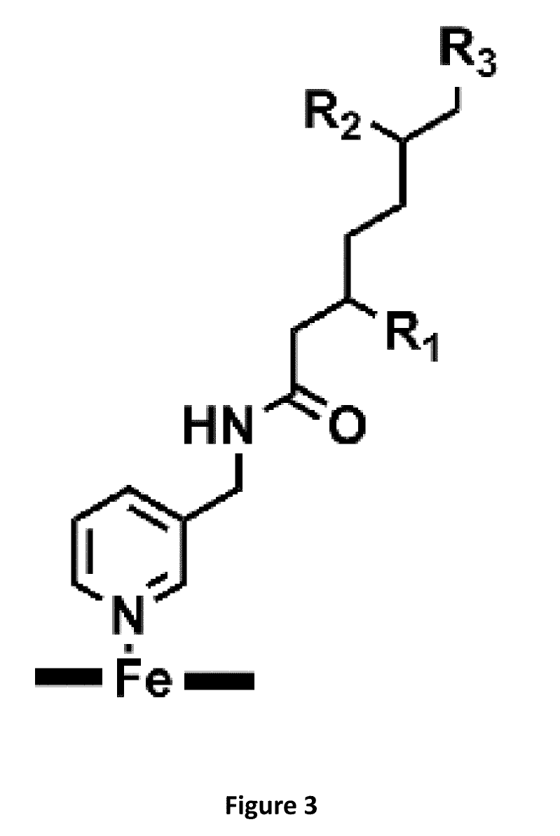

The strategy for the model evaluation was to synthesize and analyze ritonavir-like molecules using a general scaffold (FIG. 3) where the pyridine ring serves as a heme-ligating moiety and the remainder of the inhibitor is stitched together with units containing various side and terminal groups (R¹-R³). By systematically changing R¹-R³ substituents, it can be probed how each chemical moiety contributes to the binding affinity and inhibitory potency, and how CYP3A4 adapts to changes in the ligand's structure.

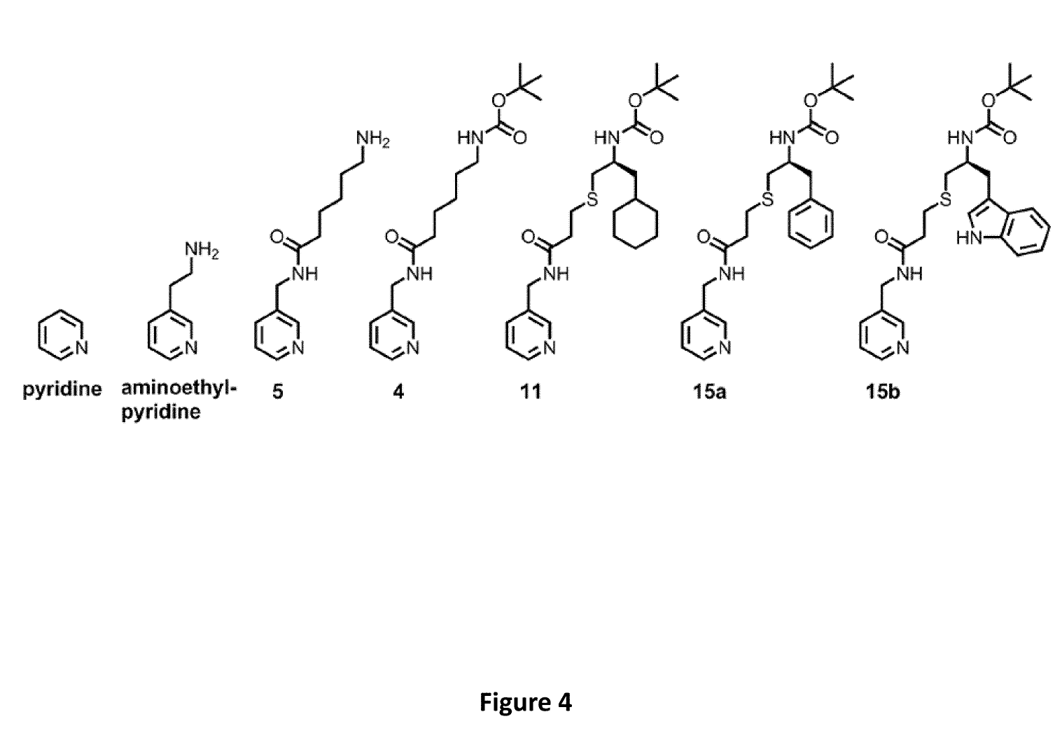

The results described herein present structural and functional results on the first set of commercially available and rationally designed pyridine-containing compounds that differ in the backbone length and R² substituents (FIG. 4). With this initial set confirms the importance of a flexible backbone, aromaticity of the R² group and the H- bond donor/acceptor moiety (pharmacophoric determinants II, III and V), as well as the leading role of hydrophobic interactions at the P1 and P2 sites in the ligand binding process.

The materials and methods employed in these experiments are now described.

### Synthesis of Analogs

### Synthesis of Compound 2

Di-tert-butyloxycarbonyl, (Boc)₂O (1.99 g, 9.14 mmol) was added to a solution of 6-aminohexanoic acid (1 g, 7.61 mmol) and NaOH (10% aqueous solution) in 1,4-dioxane. The reaction mixture was then stirred overnight at room temperature. The solvent was evaporated and the mixture was acidified using 1N HCl. The acidified mixture was then extracted with dichloromethane (DCM). The organic layer was washed with brine, dried over Na₂SO₄, filtered and concentrated. The crude material was then purified using column chromatography to give the pure product 2 (1.57 g, 89%).

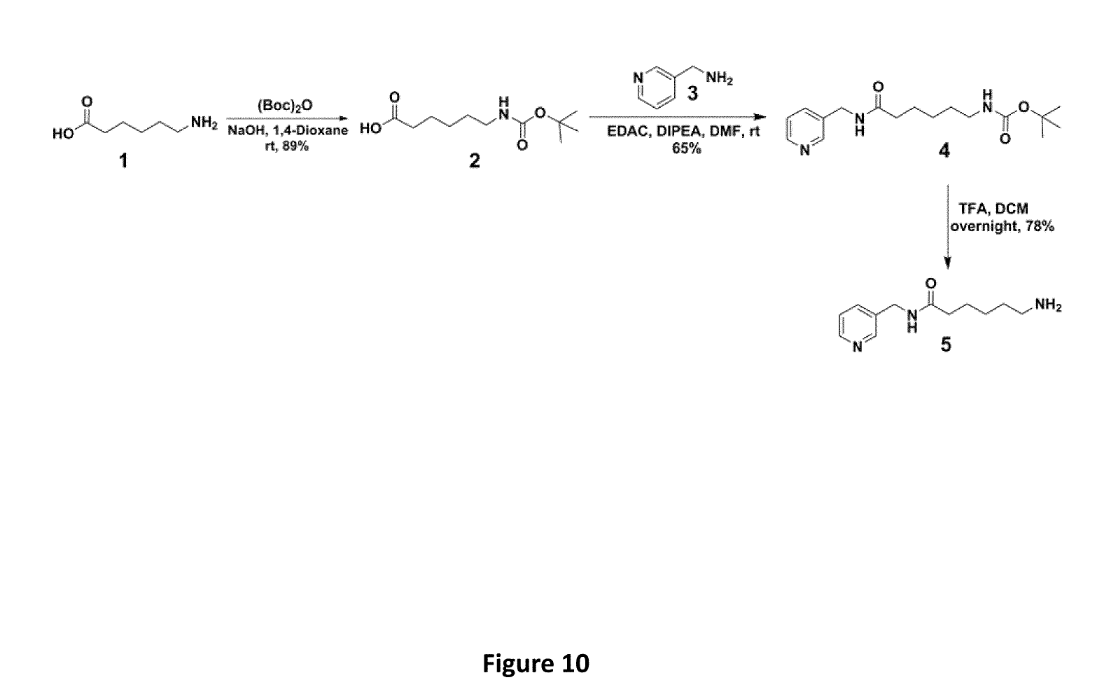

### Synthesis of Compounds 4 and 5 (FIG. 10)

Compound 2 (1 g, 4.23 mmol) was added to a solution of 1-ethyl-3-(3-dimethylaminopropyl)carbodiimide (EDAC) (1.67 g, 10.80mmol) in dimethylformamide (DMF). The mixture was stirred at room temperature for 30 min after which diisopropylethylamine (DIPEA) (3.76 mL, 21.61 mmol) and 3-(aminomethyl)pyridine, 3 (0.70 g, 6.34 mmol) were added to the reaction mixture. The reaction was allowed to stir for 16 h. On completion, the solvent was evaporated and the reaction mixture was diluted with ethyl acetate. The organic layer was then washed with saturated NaHCO₃, water and brine. The combined organic layer was dried over Na₂SO₄ and concentrated to give the crude product which was then purified using column chromatography (ethyl acetate:methanol). The pure product 4 was obtained as white solid in 65% yield (0.91 g). ¹H NMR (CDCl₃, 500 MHz) δ 8.53 (s, 2H), 7.64 (d, J=7.82 Hz, 1H), 7.27 (t, J=4.80 Hz, 1H), 5.95 (bs, 1H), 4.56 (bs, 1H), 4.46 (d, J=5.91 Hz, 2H), 3.10 (q, J=6.45 Hz, 2H), 2.23 (t, J=7.45 Hz, 2H), 1.68 (quin, J=7.5 Hz, 2H), 1.50-1.46 (m, 2H), 1.42 (s, 9H), 1.34 (m, 2H); ¹³C NMR (CDCl₃, 125 MHz) δ 149.1, 148.8, 135.8, 123.7, 41.0, 36.4, 29.8, 28.4, 26.3, 25.2; HRMS m/z calculated for C₁₇H₂₇N₃O₃ [M+Na]⁺: 344.21, found: 344.2.

Compound 4 (0.6 g, 1.86 mmol) was treated with trifluoroacetic acid in DCM for 14 h at room temperature in order to remove the Boc-group. The completion of the reaction was monitored through TLC. The reaction solvent was evaporated and the crude product was passed through celite to obtain the pure amine 5 (0.47 g, 78% yield). ¹H-NMR (500 MHz, CD₃OD) δ 8.81 (1H, s), 8.79-8.78 (2H, d, J=5.5 Hz), 8.53-8.51 (1H, d, J=8.3 Hz), 8.06-8.03 (1H, m), 2.97-2.91 (2H, m), 2.37-2.34 (2H, t, J=7.4 Hz), 1.72-1.68 (4H, m), 1.48-1.39 (4H, m); ¹³C NMR (CD₃OD, 125 MHz) δ 175.2, 161.1, 144.5, 142.4, 139.2, 126.4, 39.7, 38.9, 34.3, 29.8, 26.6, 24.3. HRMS m/z calculated for C₁₂H₁₉N₃O [M+H]⁺: 222.23, found: 222.2.

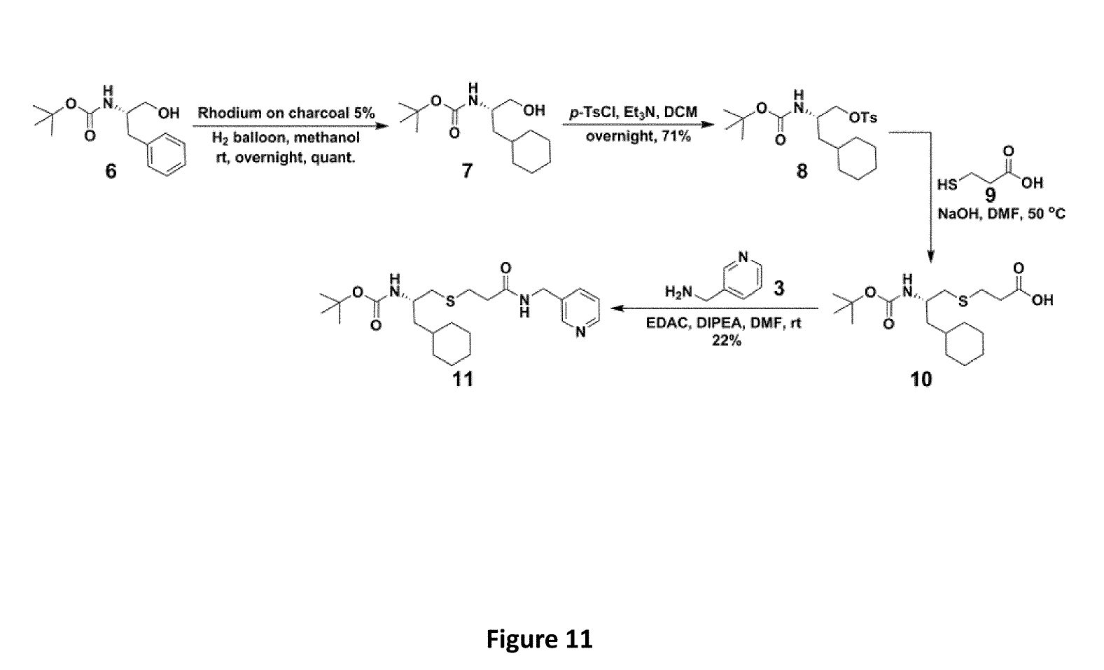

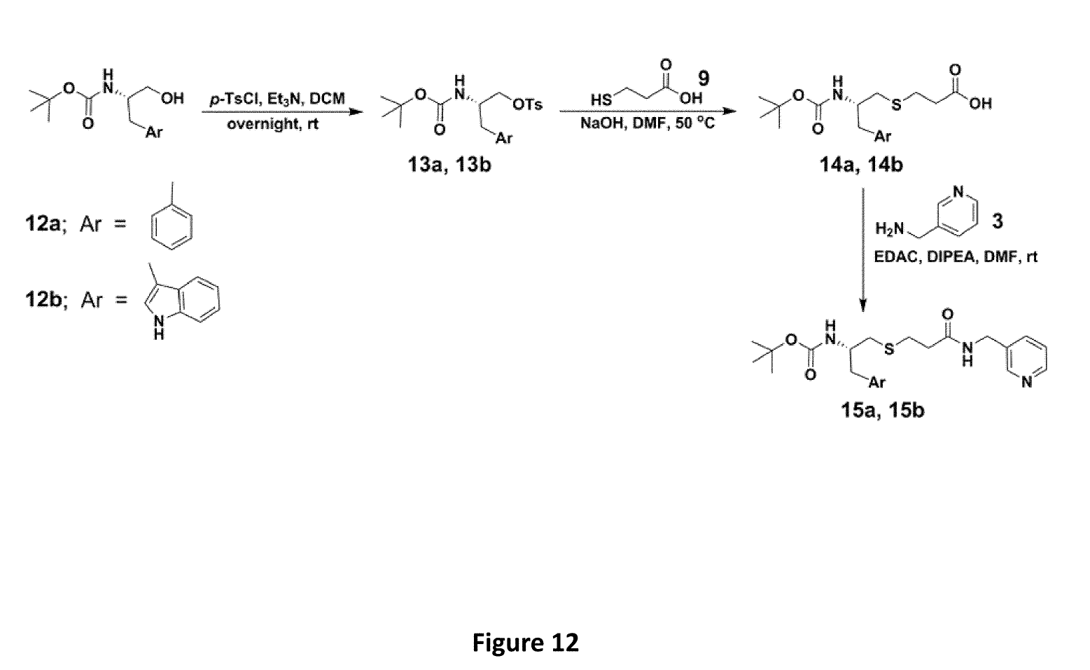

After analyzing different conditions and routes, synthesis of the first generation analogs was started using (S)-Boc-phenylalaninol as shown in FIGS. 11 and 12. (S)-Boc-phenylalaninol or other corresponding amino alcohols (shown in the schemes) were converted to a tosylate using p-toluenesulphonyl chloride, which was then reacted with 3-mercaptopropionic acid in the presence of NaOH at 50° C. to give the corresponding acid. The acid was then coupled to the 2-(3-pyridyl)ethylamine using the known usual EDAC coupling to give the corresponding final product.

### Synthesis of Compound 7

S-Boc-phenylalaninol (2 g, 7.96 mmol) was dissolved in methanol and treated with rhodium on charcoal (5%; 100 mg) under H₂ atmosphere overnight to reduce the phenyl ring. The solution was then passed through celite plug, and methanol was evaporated to give the pure product 7 in quantitative yield (2.01 g, quant).

### Synthesis of Compound 8

S-Boc-cyclohexylalaninol (2 g, 7.78 mmol) was dissolved in DCM. To this solution, triethylamine (3.25 mL, 23.34 mmol) andp-toluenesulphonyl chloride (2.23 g, 11.67 mmol) were added slowly at 0° C. The reaction was then allowed to stir at room temperature overnight. After reaction completion, DCM was evaporated and the crude reaction mixture was purified using column chromatography. The pure product 8 was obtained as white fluffy solid (2.3 g, 71% yield). LCMS m/z calculated for C₂₁H₃₃NO₅S [M+Na]⁺: 434.21, found 434.23.

### Synthesis of Compound 10

To solution of compound 8 (1 g, 2.43 mmol) in DMF, 3-mercaptopropanoic acid, 9 (0.309 g, 2.91 mmol) was added. To this mixture, 1N NaOH (2 mL) was added and reaction was allowed to stir for 5 h at 50° C. The crude product 10 was obtained by evaporating the solvent and was used for the next step without any further purification due to instability of the product on silica column. LCMS m/z calculated for C₁₇H₃₁NO₄S [M+2Na]⁺: 368.20, found 368.17.

Synthesis of compound 11 (FIG. 11)

Compound 10 (0.1 g, 0.3 mmol) was added to a solution of EDAC (0.12 g, 0.72 mmol) in DMF. The mixture was stirred at room temperature for 30 min after which DIPEA (0.23 mL, 1.4 5 mmol) and 3-(aminomethyl)pyridine, 3 (0.042 g, 0.43 mmol) were added. The reaction was allowed to stir for 18 h. On completion, the solvent was evaporated and the reaction mixture was diluted with ethyl acetate. The organic layer was then washed with saturated NaHCO₃, water and brine. The combined organic layers were dried over Na₂SO₄ and concentrated to give the crude product which was then purified using column chromatography (ethyl acetate:methanol). The pure product 11 was obtained as yellow oil in 22% yield (0.005g). ¹H-NMR (400 MHz, CD₂Cl₂) δ 9.31-9.07 (1H, m), 8.56 (2H, bs), 7.70-7.57 (2H, m), 7.33 (1H, bs), 4.54 (1H, s), 3.74-3.20 (5H, m), 3.14-3.04 (6H, m), 2.43-2.26 (1H, m), 1.67-1.51 (4H, m), 1.44-1.33 (2H, m), 1.26 (8H, s), 1.17-1.11 (3H, m), 1.04 (3H, m). ¹³C NMR (100 MHz, CD₂Cl₂) δ 132.6, 130.5, 64.4, 32.0, 31.6, 29.9, 29.3, 27.0, 24.7, 23.4, 22.9, 22.5, 15.4, 15.2, 14.6, 13.8, 12.8, 12.0. LCMS m/z calculated for C₂₃H₃₇N₃O₃S [M+Na]⁺: 458.22, found 458.21.

### General Procedure for Synthesis of Compounds 15a and 15b

### Synthesis of Compound 13a

S-Boc-phenylalaninol (1 g, 3.98 mmol) was dissolved in DCM. To this solution, triethylamine andp-toluenesulphonyl chloride were added slowly at 0° C. The reaction was then allowed to stir at room temperature overnight. After reaction completion, DCM was evaporated and the crude reaction mixture was purified using column chromatography. The pure product 13a was obtained as white fluffy solid in 78% yield (1.26 g). The pure product 13b was obtained as white solid in 81% yield.

### Synthesis of Compound 14a

To the solution of compound 13a (0.5 g, 1.23 mmol) in DMF, 3-mercaptopropanoic acid, 9 (0.157 g, 1.48 mmol) was added. To this mixture, 1N NaOH (2 mL) was added and reaction was allowed to stir at 50° C. for 5 h. The crude product 14a was obtained by evaporating the solvent and was used for the next step without any further purification. LCMS m/z calculated for 14a: C₁₇H₂₅NO₄S [M+Na]⁺: 362.15, found 362.17. LCMS m/z calculated for 14b: C₁₉H₂₆N₂O₄S [M−H+]: 378.20, found 377.14.

### Synthesis of Compound 15a (FIG. 12)

Compound 14a (0.5 g, 1.47 mmol) was added to a solution of EDAC (0.57 g, 3.67 mmol) in DMF. The mixture was stirred at room temperature for 30 min after which DIPEA (1.28 mL, 7.35 mmol) and 3-(aminomethyl)pyridine, 3 (0.24 g, 2.21 mmol) were added. The reaction was allowed to stir for 18 h. On completion, the solvent was evaporated and the reaction mixture was diluted with ethyl acetate. The organic layer was then washed with saturated NaHCO₃, water and brine. The combined organic layers were dried over Na₂SO₄ and concentrated to give the crude product which was then purified using column chromatography (ethyl acetate:methanol). The pure product 15a was obtained as white solid in 62% yield (0.39g). ¹H-NMR (CDCl3, 500 MHz) δ 8.54 (d, J=14.4 Hz, 2H), 7.66 (d, J=9.4 Hz, 1H), 7.31-7.16 (m, 6H), 6.69 (bs, 1H), 4.72 (d, J=10.3 Hz, 1H), 4.52-4.40 (m, 2H), 2.87 (t, J=9.3 Hz, 3H), 2.63-2.51 (m, 4H), 1.40 (d, J=4.15 Hz, 2H), 1.36 (s, 9H); ¹³C NMR (CDCl3, 125 MHz) δ 149.2, 148.8, 135.6, 129.4, 129.3, 128.8, 128.6, 126.7, 123.6, 41.0, 36.6, 28.6, 28.3; HRMS m/z calculated for C₂₃H₃₁N₃O₃S [M+H]⁺: 430.231, found 430.21.

The pure product 15b was obtained as yellow oil in 56% yield. ¹H-NMR (CDCl₃, 500 MHz) δ 8.51 (bs, 2H), 8.25 (s, 1H), 7.64 (d, J=7.59 Hz, 2H), 7.35 (d, J=8.07 Hz, 1H), 5.95 (bs, 1H), 7.23 (t, J=4.85 Hz, 1H), 7.18 (t, J=7.58 Hz, 1H), 7.11 (t, J=7.28 Hz, 1H), 7.02 (s, 1H), 6.54 (s, 1H), 4.76 (bs, 1H), 4.41 (t, J=5.91 Hz, 2H), 4.05 (m, 1H), 3.01 (bs, 2H), 2.86 (bm, 2H), 2.62 (bm, 2H), 2.49 (bm, 2H), 1.36 (s, 9H); ¹³C NMR (CDCl₃, 125 MHz) δ 155.7, 149.1, 148.7, 136.3, 135.7, 134.1, 129.4, 126.6, 123.6, 122.9, 122.2, 119.7, 119.0, 111.3, 111.2, 50.7, 41.0, 36.5, 35.8, 28.8, 28.4.; HRMS m/z calculated for C₁₇H₂₇N₃O₃ [M+Na]⁺: 491.21, found 491.2.

The purity of compounds 4, 5, 15a and 15b was >95% as determined by ¹H NMR analysis. The purity of compound 11, however, did not exceed 90% even after repeated attempts of column chromatography purification.

### Protein Expression and Purification

The K282A/E285A mutation was introduced to the CYP3A4Δ3-22 expression plasmid using a QuikChange mutagenesis kit (Stratagene). The C-terminally 4-histidine tagged wild type and mutant CYP3A4 were produced, purified and quantified as reported previously (Sevrioukova and Poulos, 2010, Proc Natl Acad Sci USA 107:18422-7).

### Spectral Binding Titrations

Ligand binding to CYP3A4 was monitored in 50 mM phosphate, pH 7.4, containing 20% glycerol and 1 mM dithiothreitol (buffer A) in a Cary 300 spectrophotometer. The protein was titrated with small aliquots of the investigated compounds dissolved in dimethyl sulfoxide, with the final solvent concentration <2%. Spectral dissociation constants (K_{S}) were determined from the titration curves using one- or two-site saturation fitting.

### Kinetics of Ligand Binding

Kinetics of ligand binding to CYP3A4 was monitored at room temperature in 50 mM phosphate, pH 7.4, in a SX.18MV stopped flow apparatus (Applied Photophysics,

UK) after mixing 2 μM CYP3A4 with various concentrations of ligands. Absorbance changes were followed at 409-411 nm for pyridine, aminoethylpyridine and compound 5, and at 427-428 nm for 4, 11, and 15a-b. Kinetic data were analyzed using the program IgorPro (WaveMetrics, Inc).

### Thermal Denaturation and Inhibitory Potency Assays

Thermal denaturation experiments were conducted as previously described (Sevrioukova and Poulos, 2013 J. Med. Chem. 56: 3733-41) to compare ligand-dependent changes in CYP3A4 stability. The inhibitory potency of the investigated compounds on the 7-benzyloxy-4-(trifluoromethyl)coumarin (BFC) O-debenzylation activity of CYP3A4 was evaluated fluorometrically in a reconstituted system with rat cytochrome P450 reductase (CPR). The reaction was carried out at room temperature in 100 mM phosphate buffer, pH 7.4, containing catalase and superoxide dismutase (2 Units/ml each). A mixture of 1 μM CYP3A4 and 1 μM CPR was preincubated for 1 hour at room temperature and diluted by 20-fold with the assay buffer immediately before measurements. BFC (50 μM) and various concentrations of analogs were added to the protein mixture 2 minutes prior to the reaction initiation with 100 μM NADPH. Formation of 7-hydroxy-4-trifluoromethylcoumarin was followed in a Hitachi F100 fluorometer using λₑₓ=430 nm and λₑₘ=500 nm. IC₅₀ values were derived from the [% activity] vs. [inhibitor] plots.

### Crystallization and Determination of the X-ray Structures of Ligand-bound CYP3A4

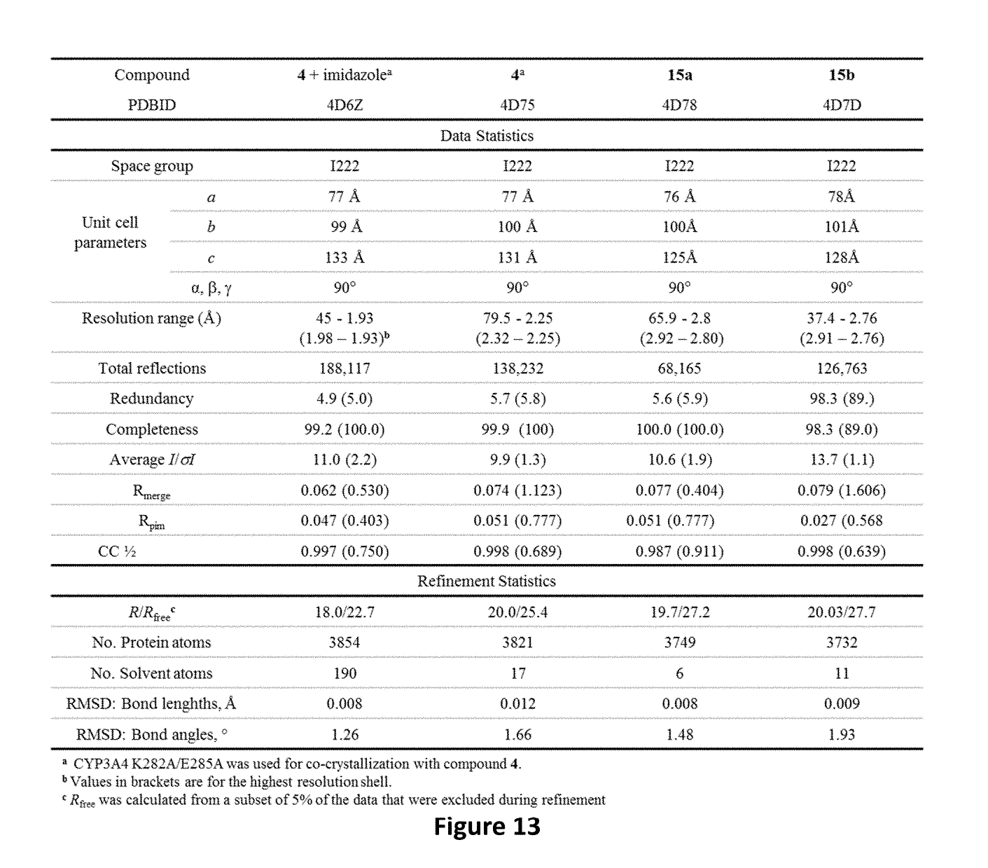

CYP3A4 K282A/E285A was co-crystallized with 4 by a sitting drop vapor diffusion method. Protein (80 mg/ml) in buffer A was mixed with a 10-fold excess of 4 and centrifuged to remove the precipitate. CYP3A4 K282A/E285A directly ligated to 4 was crystallized against the 80% solution E7 from the Morpheus crystallization screen (Molecular Dimensions). The imidazole-containing solution A3 from the same screening kit resulted in co-crystallization of the mutant with both imidazole and 4 in the active site. Crystals of the wild type CYP3A4 bound to compounds 15a and 15b were grown by a microbatch method under oil. A half of a microliter of ligand-bound CYP3A4 was mixed with 0.5 μl of 6-10% polyethylene glycol 3,350 and 60-90 mM sodium malonate, pH 5.0, and covered with paraffin oil. All crystals were cryoprotected with Paratone-N and frozen in liquid nitrogen. X-ray diffraction data were collected at the Stanford Synchrotron Radiation Lightsource beamline 7-1 and the Advanced Light Source beamline 8.2.1. Crystal structures were solved by molecular replacement with PHASER37 and the ligand-free 1TQN structure as a search model. The initial models were rebuilt and refined with COOT38, PHENIX39 and REFMAC.37 Data collection and refinement statistics are summarized in FIG. 13. The atomic coordinates and structure factors for 4-, imidazole/4-, 15a- and 15b-bound CYP3A4 have been deposited in the Protein Data Bank with the ID codes 4D75, 4D6Z, 4D78 and 4D7D, respectively.

The results of the experiments are now described.

### Inhibitor Design

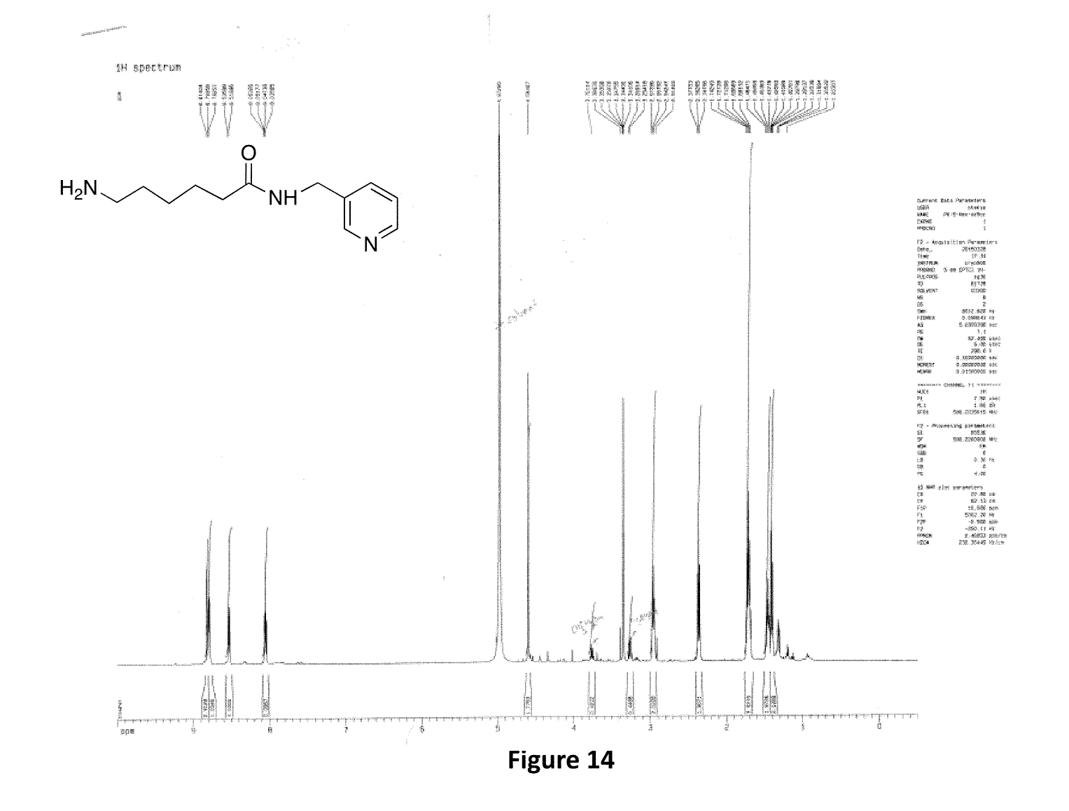

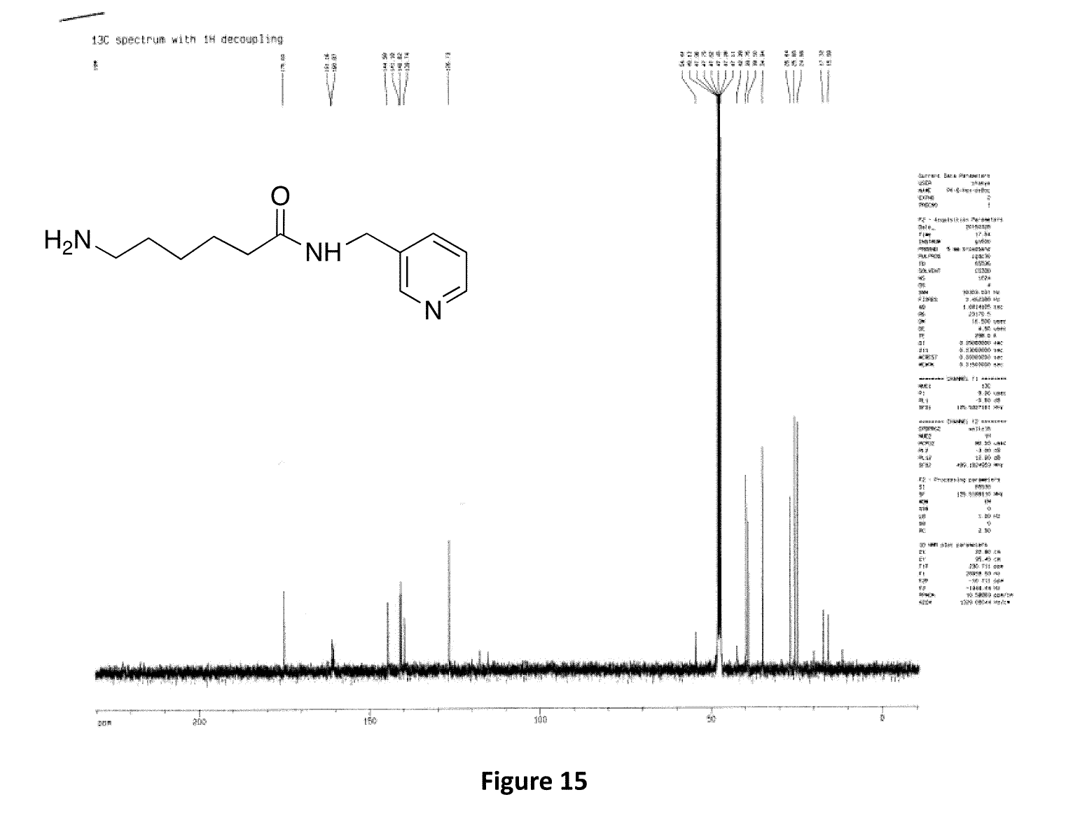

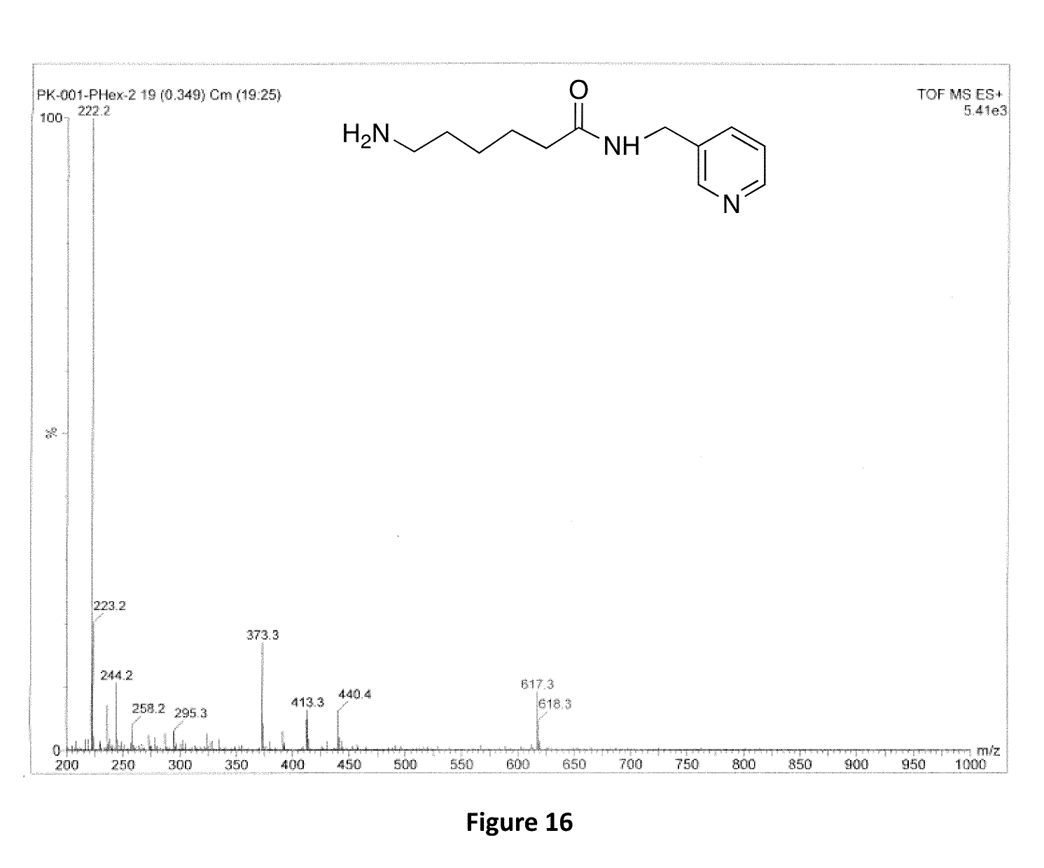

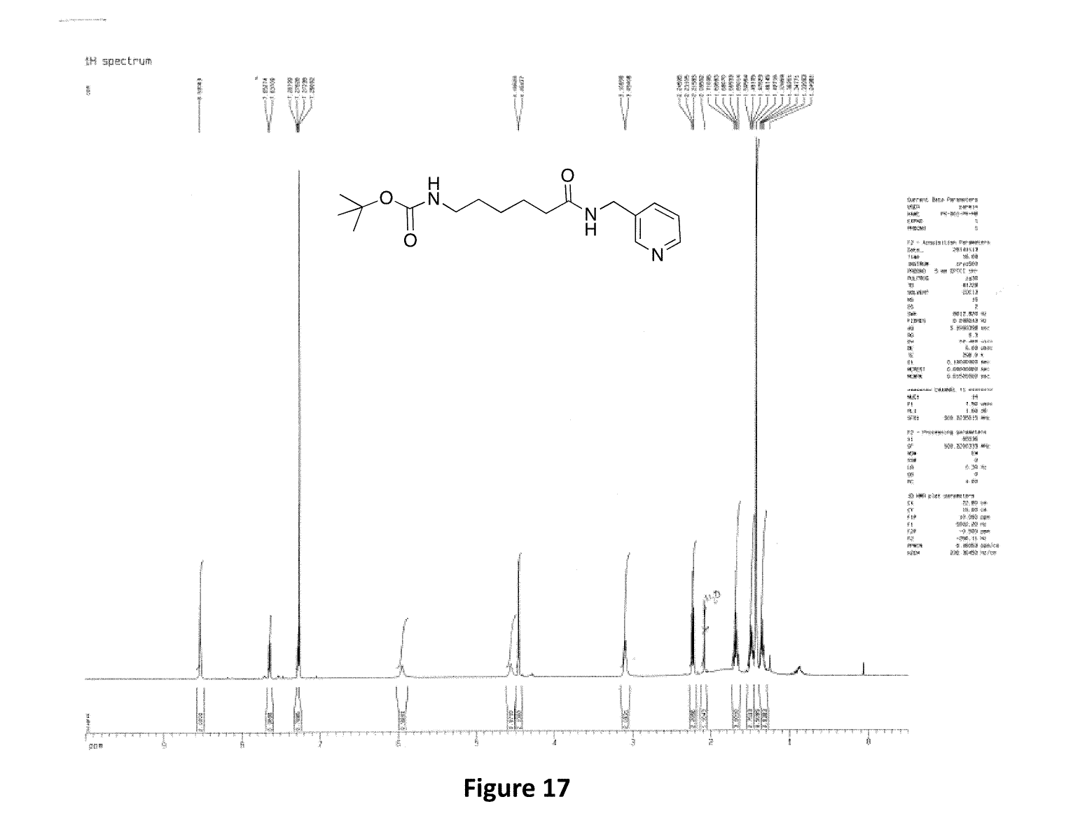

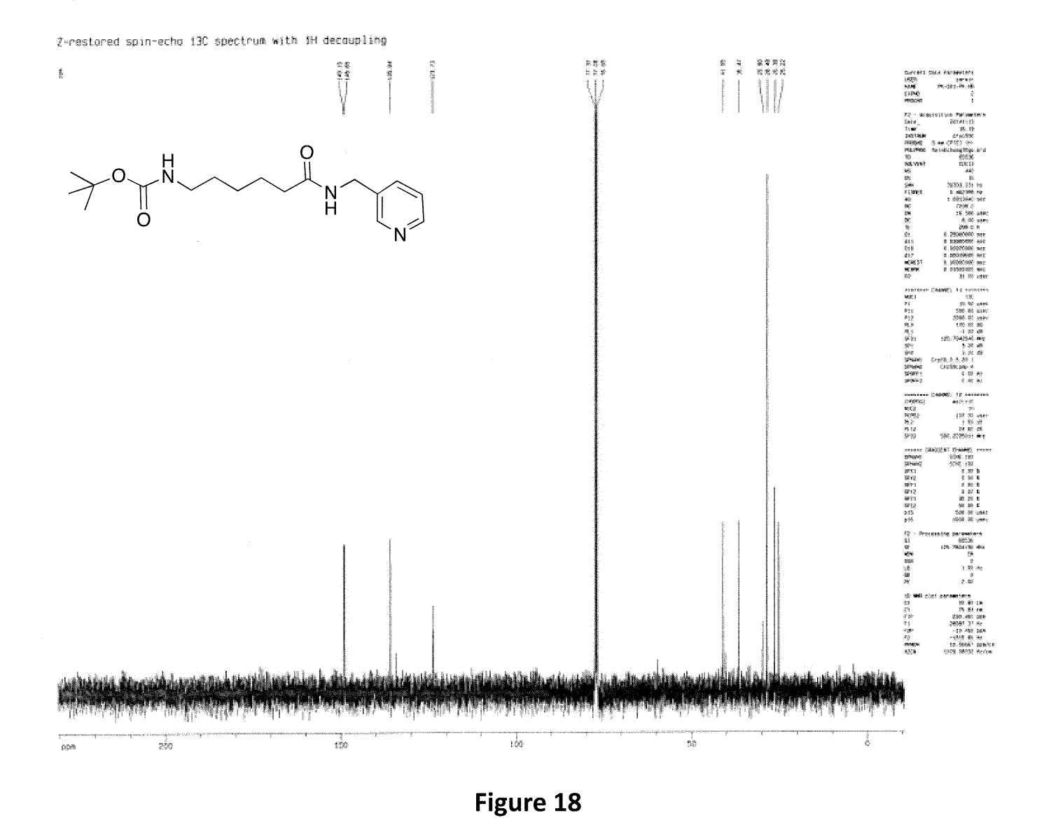

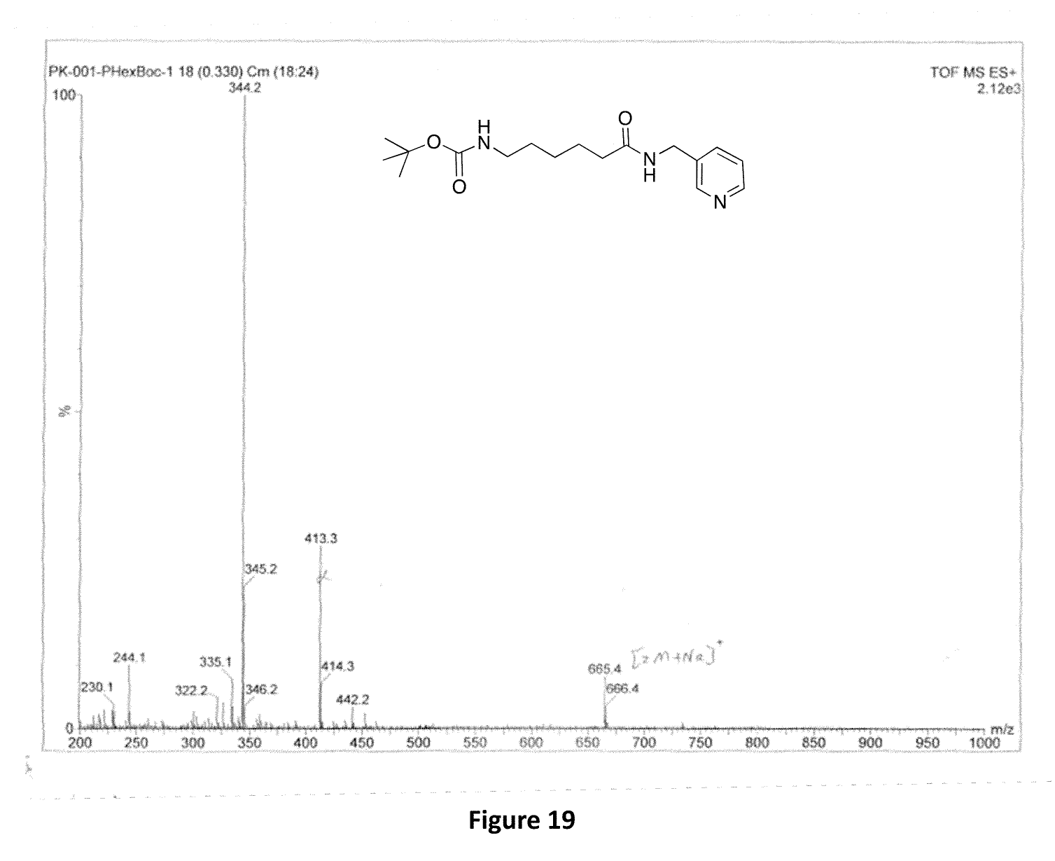

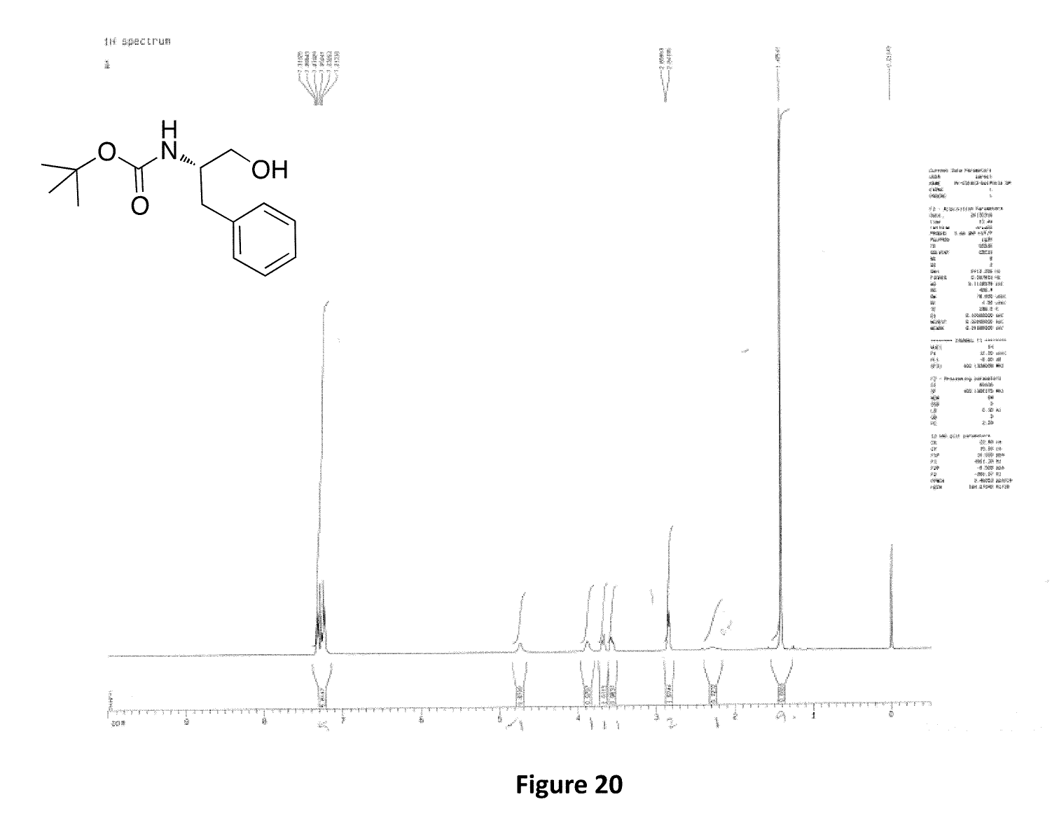

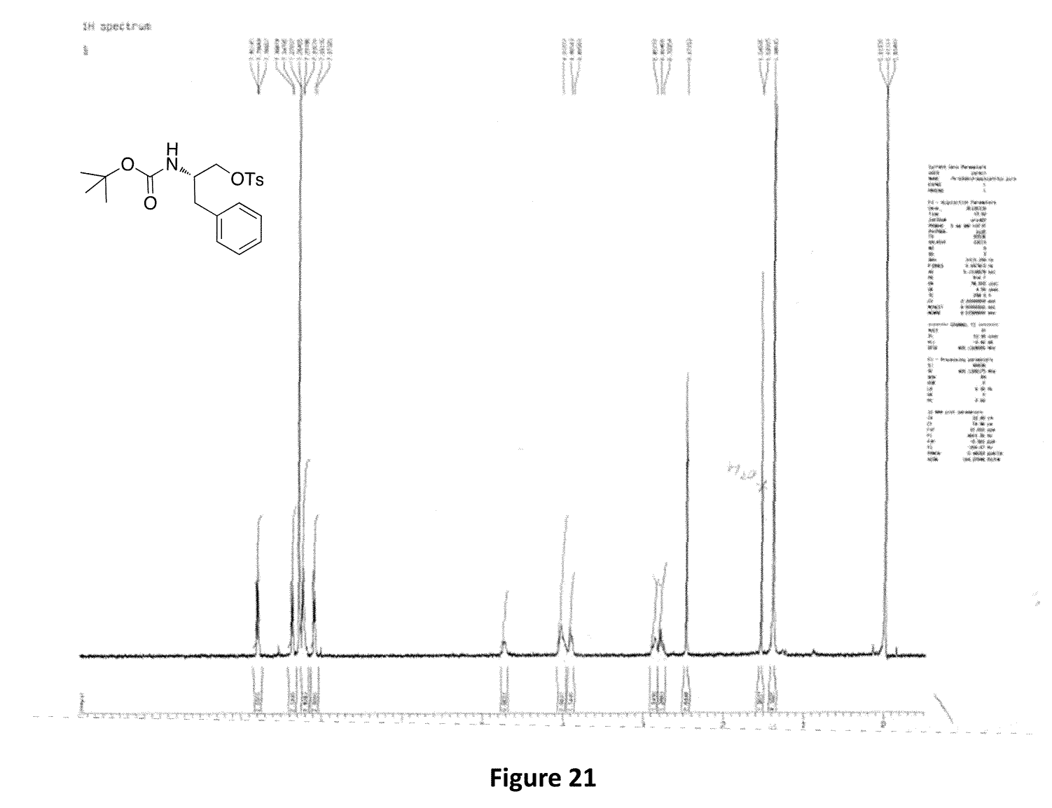

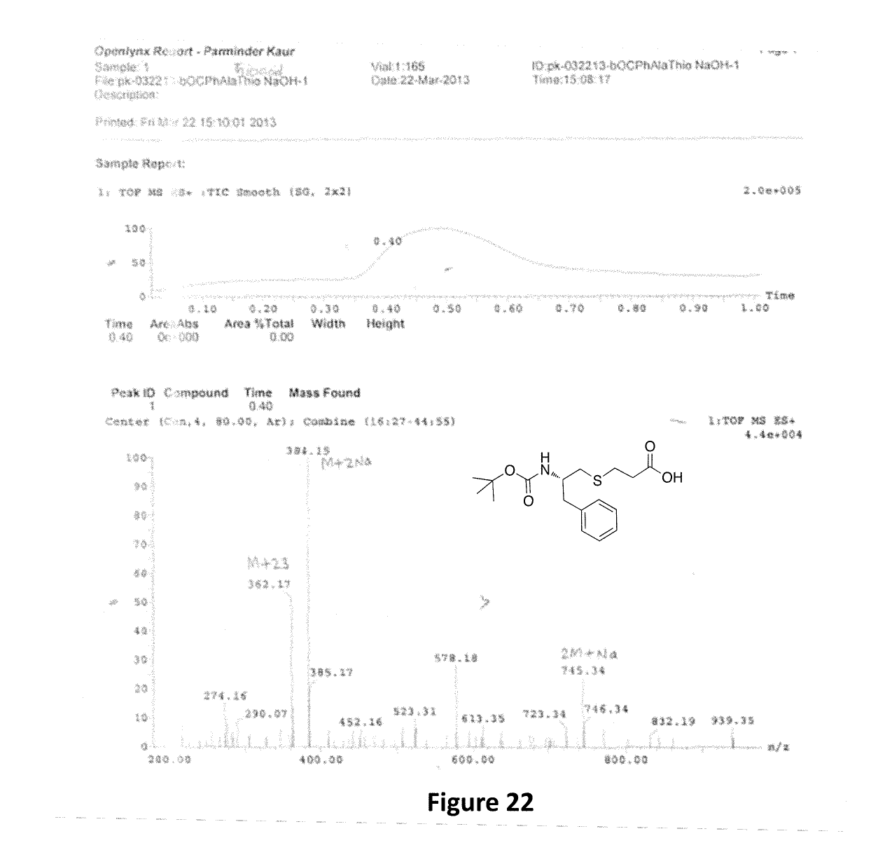

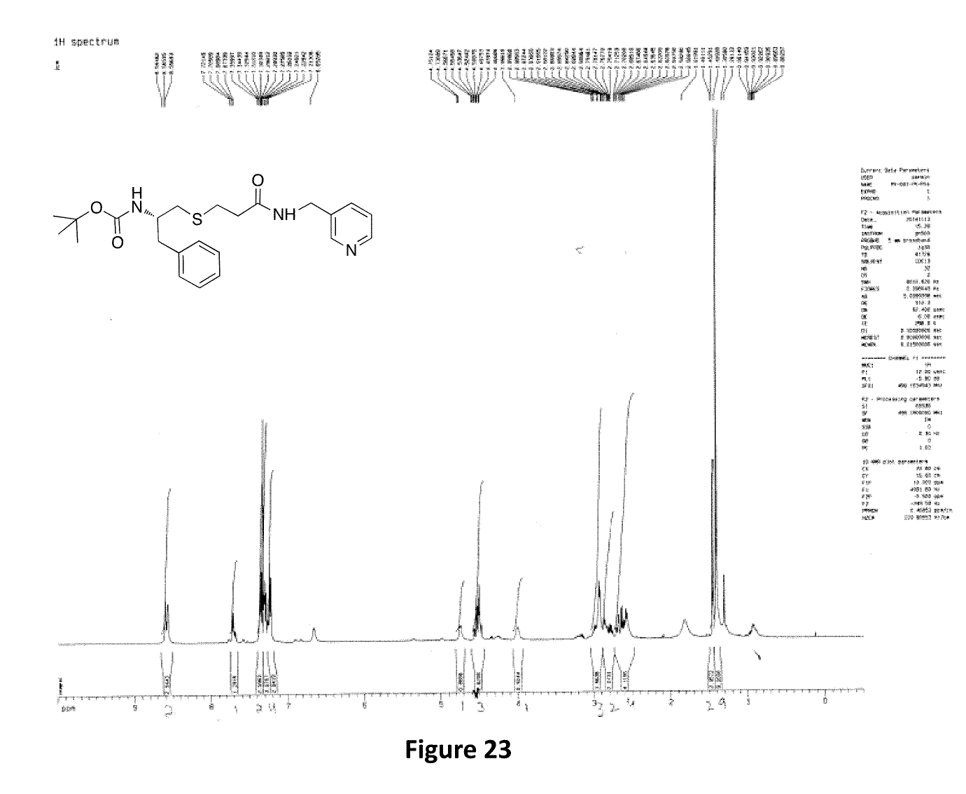

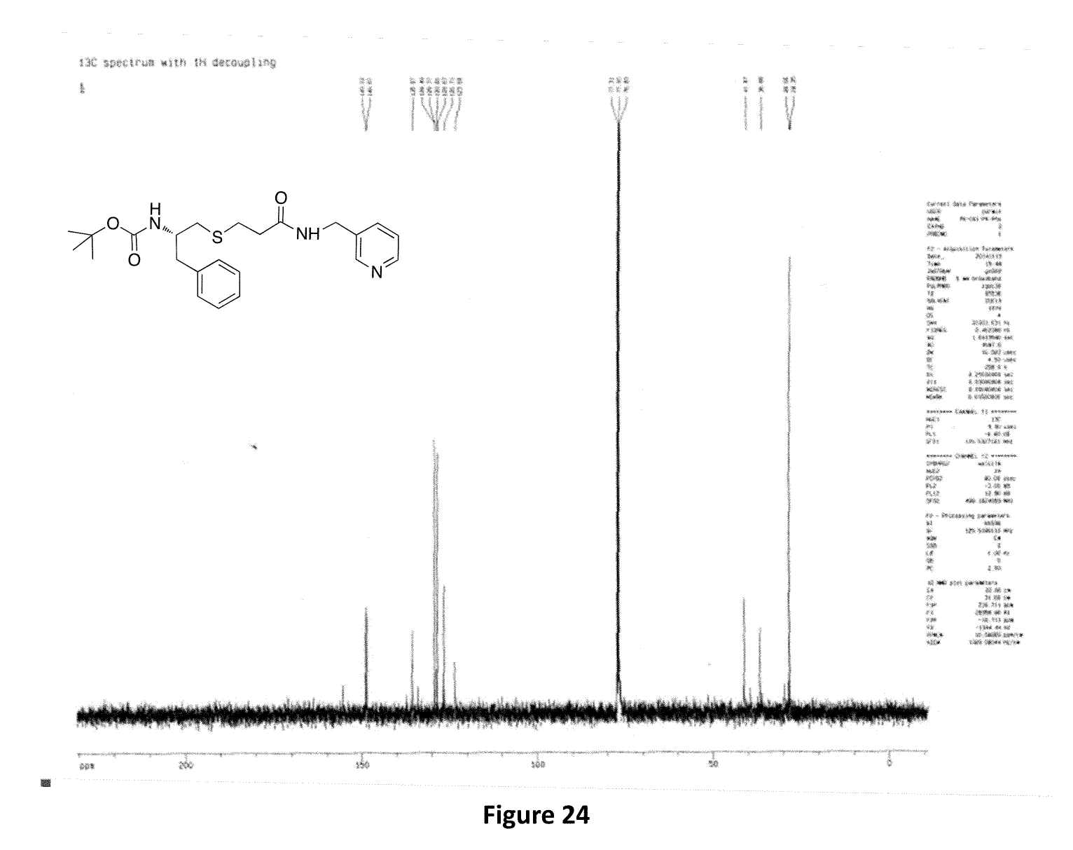

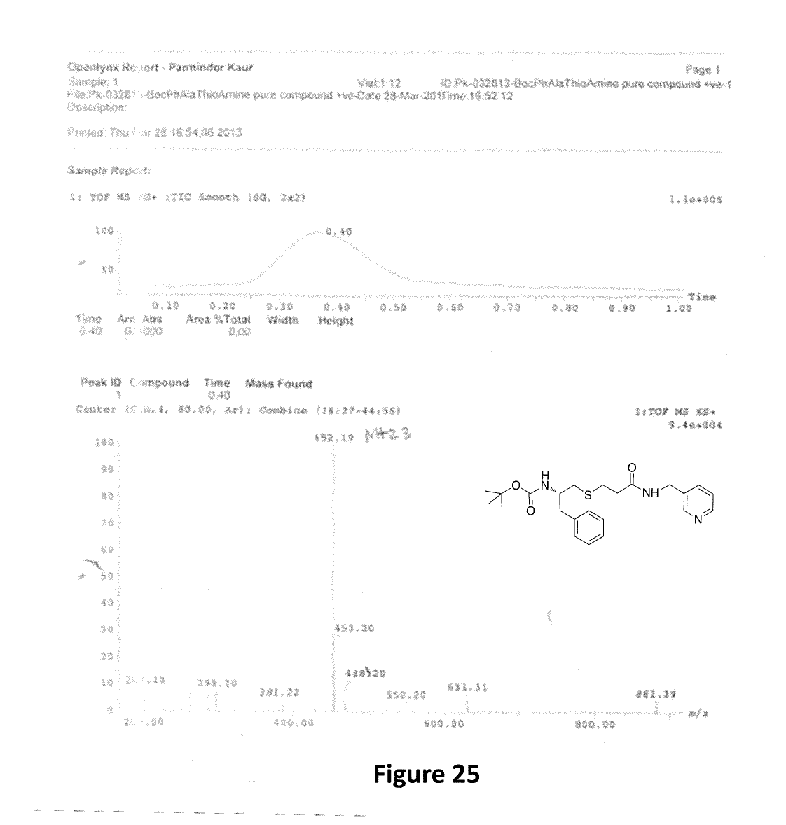

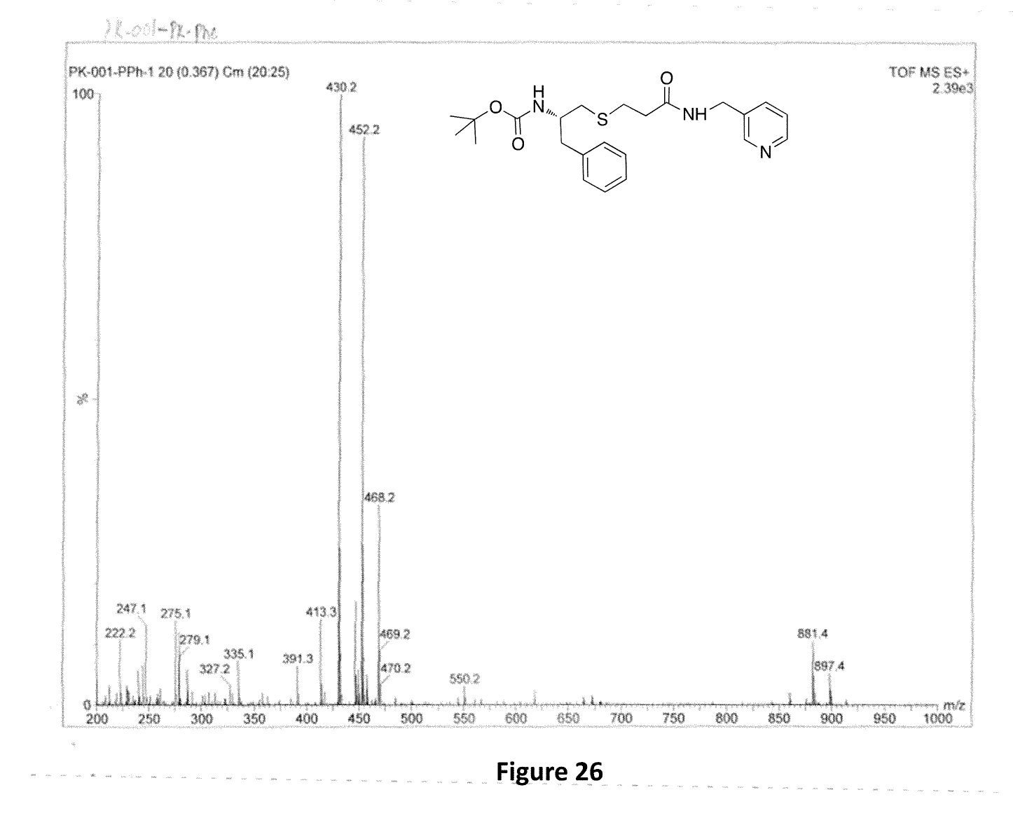

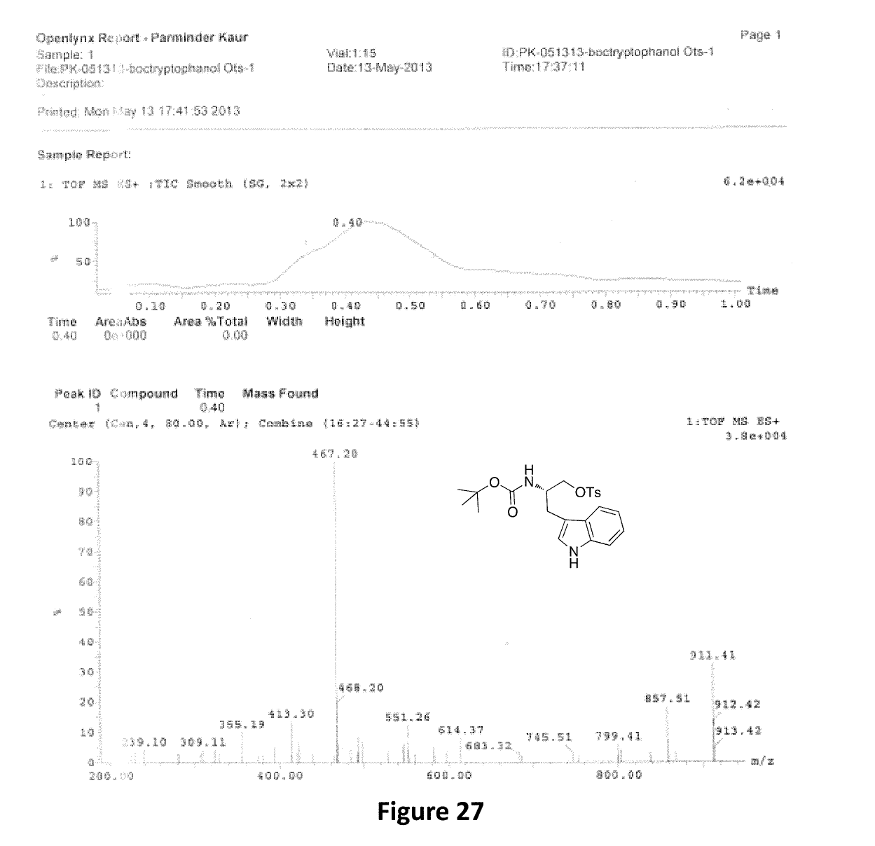

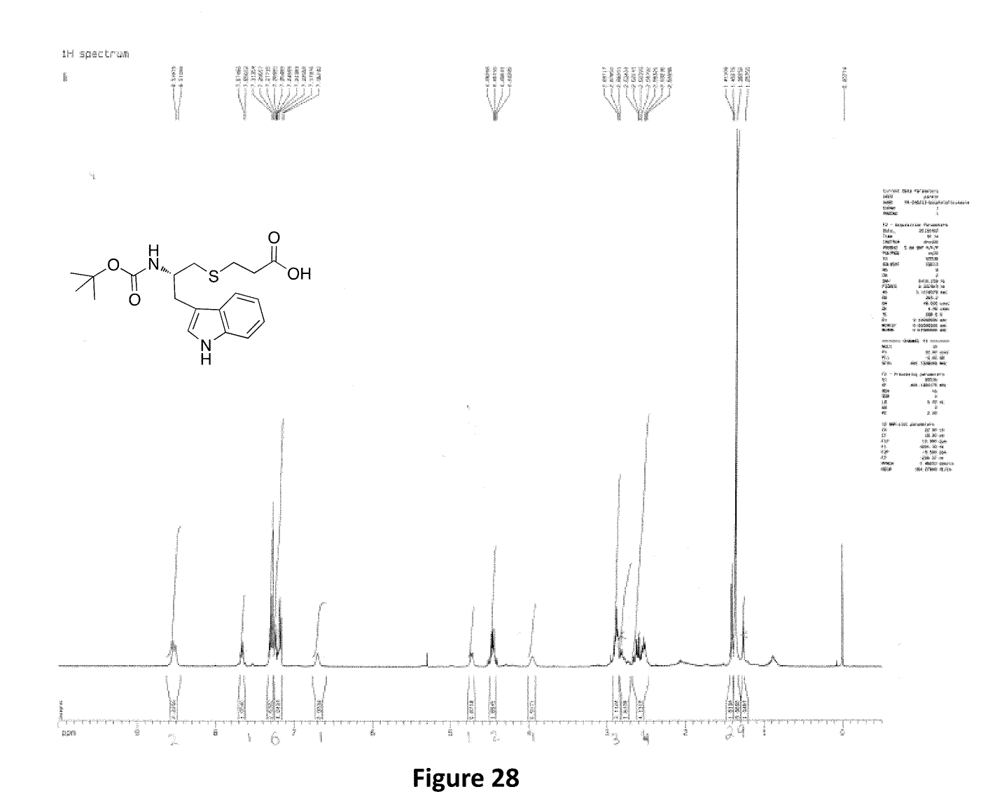

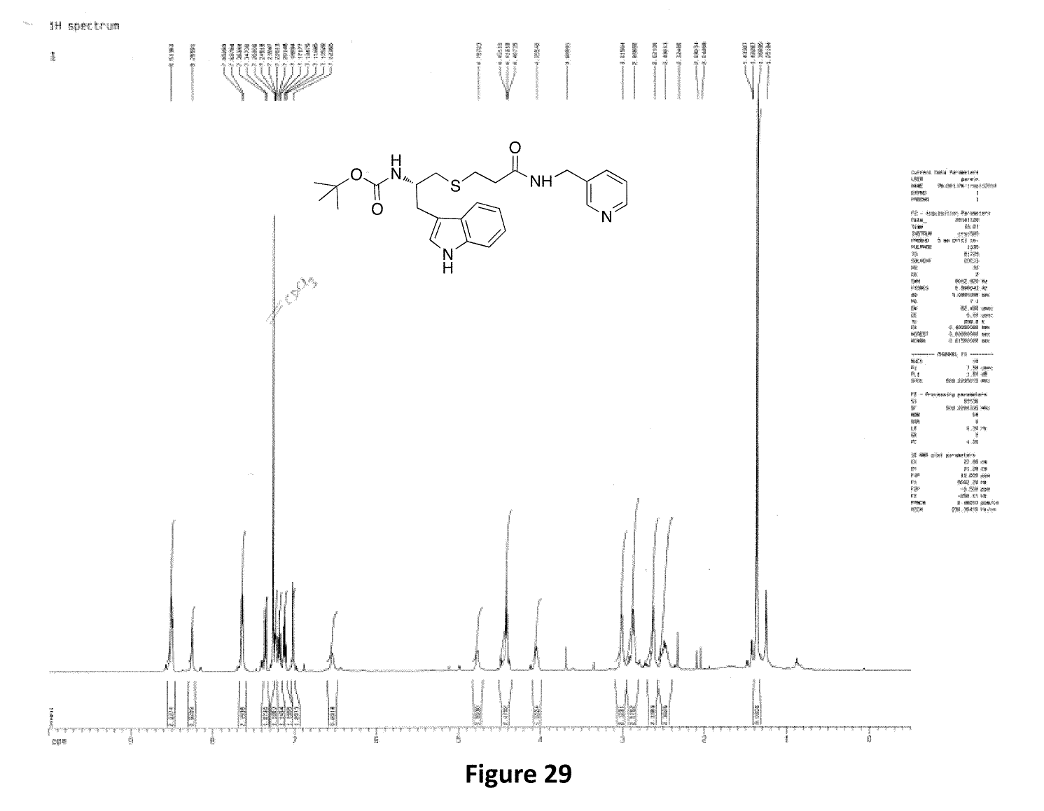

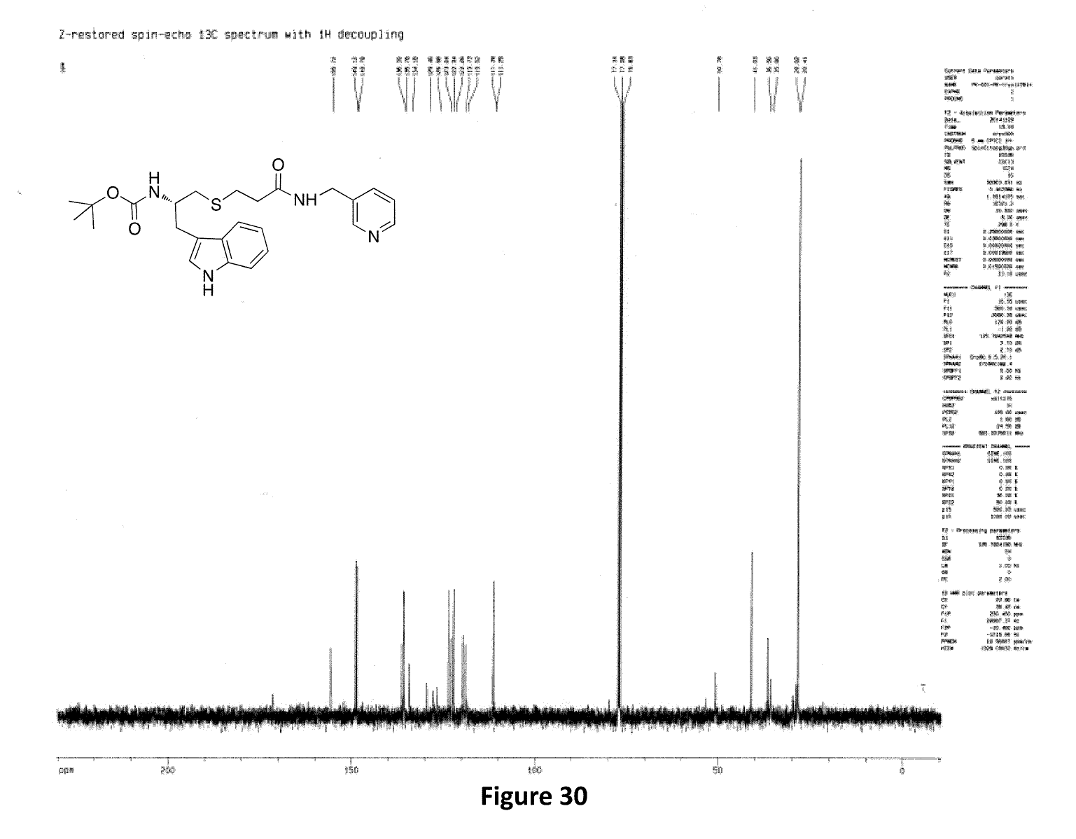

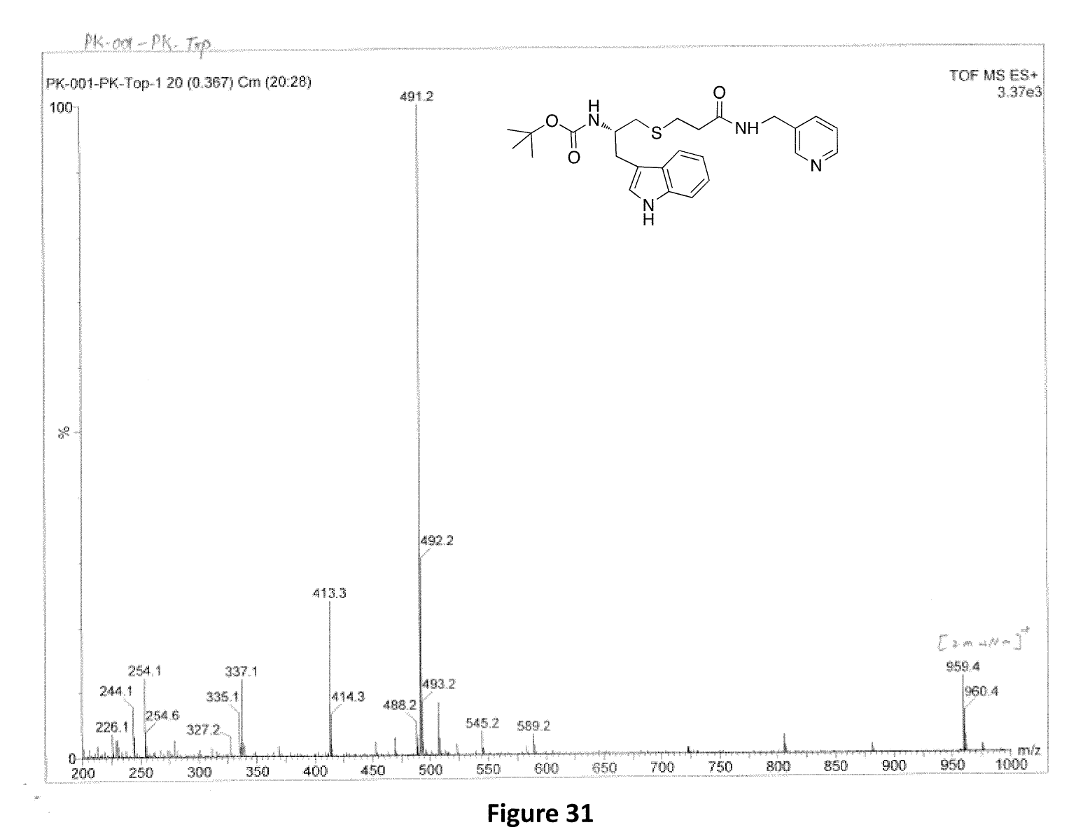

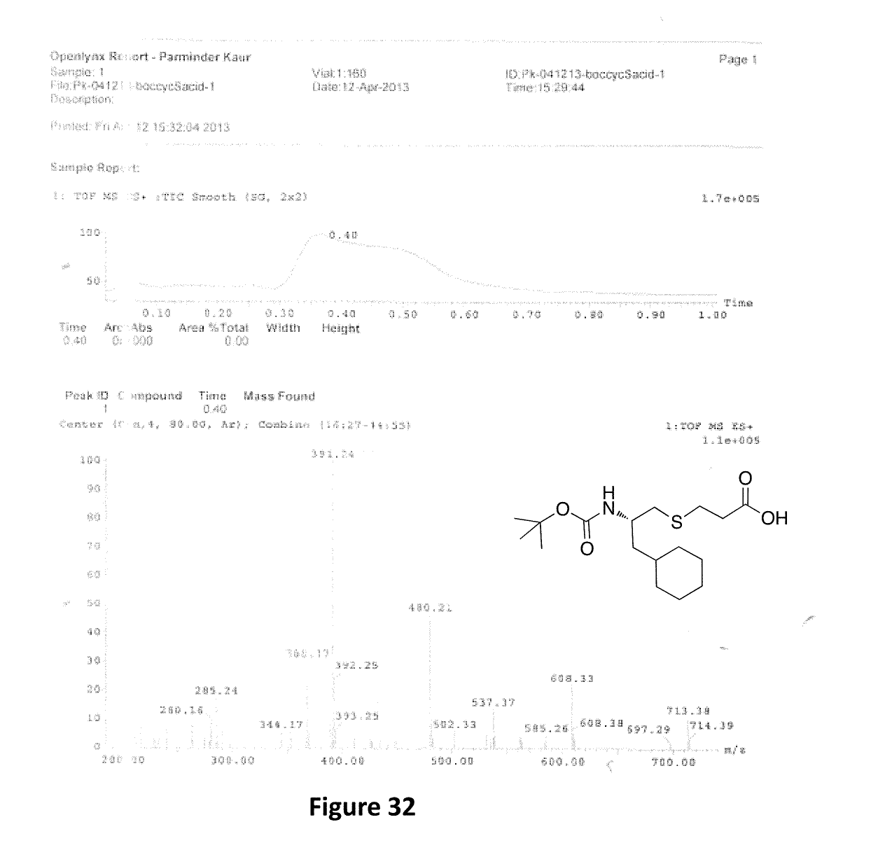

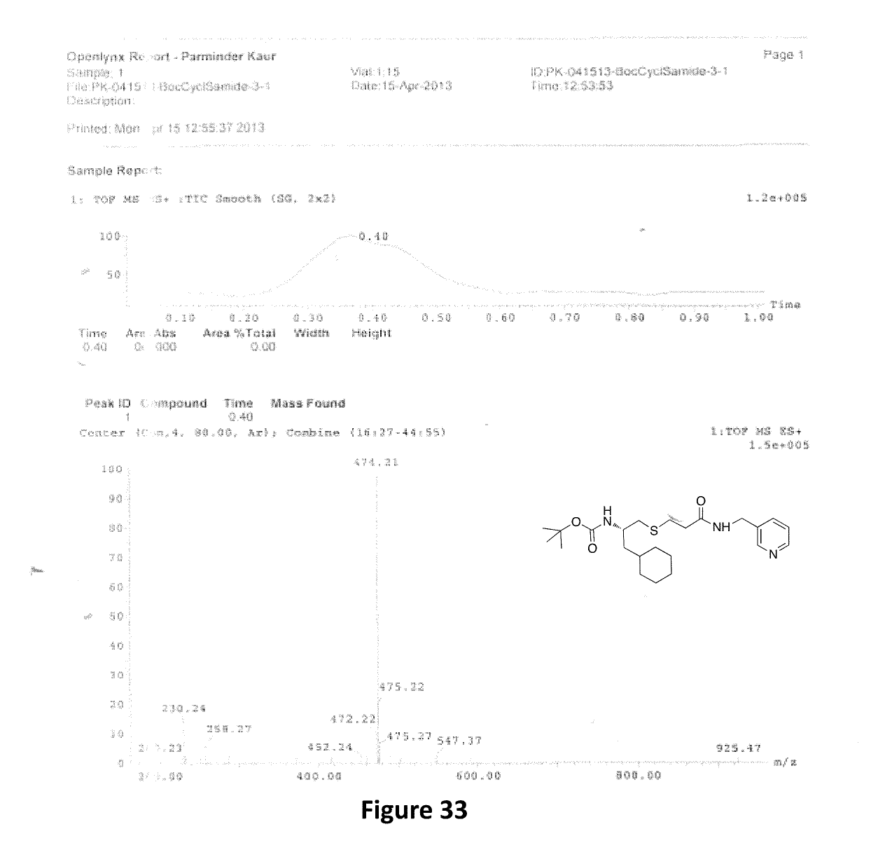

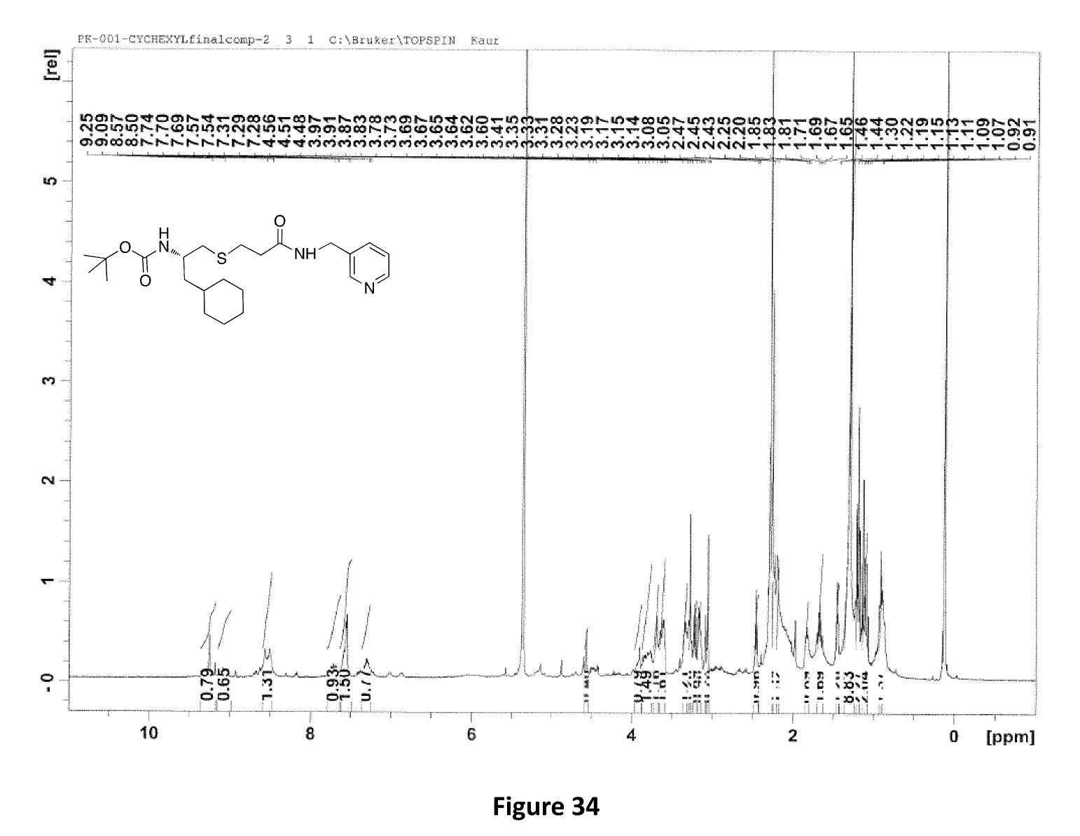

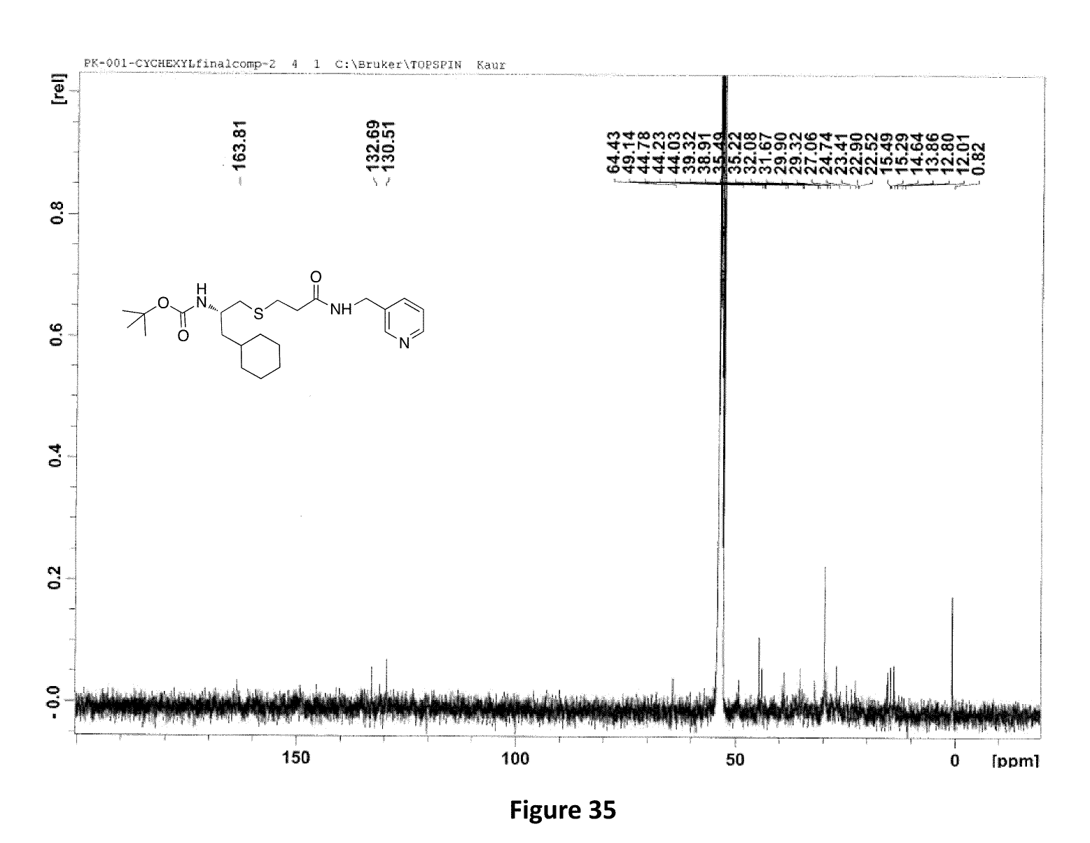

Both CYP3A4 inhibitors currently marketed as pharmacoenhancers, ritonavir and cobicistat (FIG. 1), were developed based on chemical structure/activity relationships studies rather than the CYP3A4 crystal structure. Ritonavir is a large peptidomimetic drug designed to inhibit an HIV-I protease (Kempf et al., 1997, Antibicrom Agents Chemother 41:654-60), whereas cobicistat was developed through ritonavir derivatization (Xu et al., 2014, Bioorg Med Chem Lett, 24:995-9; Liu et al., 2014, Bioorg Med Chem Lett 24:989-94). Synthesis of both compounds is a complex process requiring production of specific stereoisomers. To evaluate the pharmacophore model, CYP3A4 inhibitors were built from scratch using a general simple scaffold (FIG. 3) where the modules containing the side and terminal groups (R¹, R² and R³) are linked to the heme-ligating pyridine via a flexible backbone. Pyridine and aminoethylpyridine (FIG. 4) were obtained from Sigma-Alrdrich. The rationale for designing compounds 4, 5, 11 and 15a-b was to determine how elongation of the backbone, introduction of the H-bond donor/acceptor (peptide bond), and substitution of R² affect affinity, inhibitory potency and the ligand binding mode. Mass spectrometry and NMR data are included in FIGS. 14-35.

### Binding Affinity and Association Kinetics

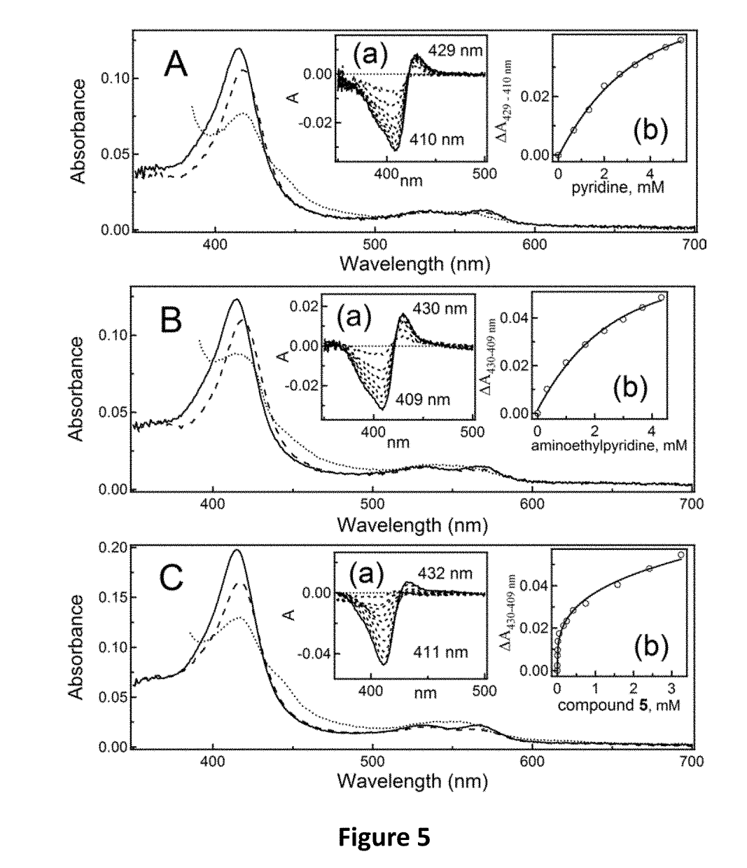

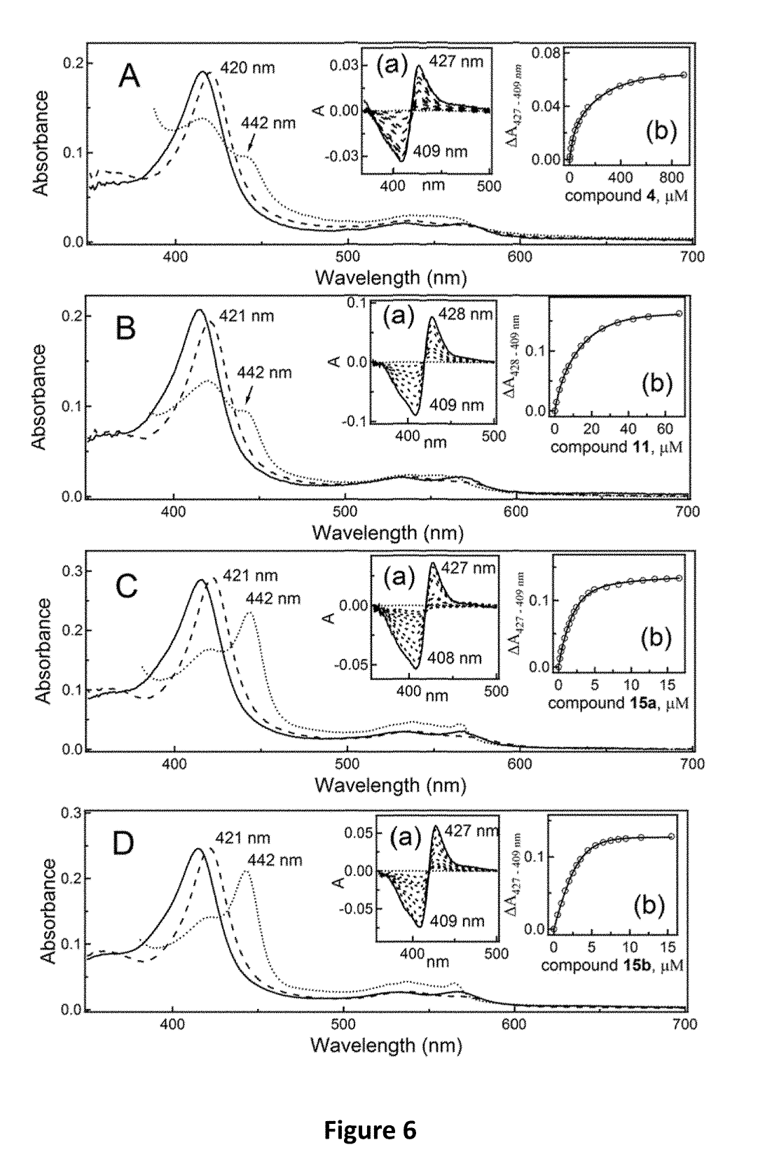

Addition of pyridine, aminoethylpyridine and compound 5 to CYP3A4 led to a decrease and a small red shift (−2 nm) in the Soret band (FIG. 5). Spectral dissociation constants (K_{S}) for pyridine and aminoethylpyridine were in the millimolar range (Table 1), whereas 5 had two binding sites with K_{S} of 10 μM and 4.4 mM. Compounds 4, 11 and 15a-b, on the other hand, bind to a single site and induce a notable shift in the Soret band, typical for type II ligands (FIG. 6). Introduction of the hexane or phenyl side group increased the binding affinity by 10- and 100-fold, respectively, relative to 4. The phenyl-to-indole substitution led to a further decline in K_{S} (by ˜2-fold; Table 1). Another important feature is a pronounced 442 nm absorption of the ferrous 15a- and 15b-bound forms of CYP3A4. The corresponding 442 nm peak was considerably smaller for the CYP3A4-4/11 complexes and undetectable for the pyridine-, aminoethylpyridine- and 5-bound forms (FIGS. 5 and 6). In previous studies (Sevrioukova and Poulos, 2010, Proc Natl Acad Sci USA 107:18422-7; Sevrioukova and Poulos, 2012, Arch biochem Biophys 520:108-16; Sevrioukova and Poulos, 2013, J Med Chem 56:3733-41; Sevrioukova and Poulos, 2013, Biochemistry 52:4474-81), it was shown that the amplitude of the 442 nm band of the ferrous species correlates with the inhibitory potency of the ligand, in that compounds whose binding led to a higher 442 nm absorption peak were acting as more potent CYP3A4 inactivators (e.g., ritonavir and pyridine-substituted desoxyritonavir (GS3; FIG. 1)). Therefore, a significant but not complete conversion to the 442 nm absorbing species taking place upon association of CYP3A4 with 15a and 15b was the first indication that these compounds may have a comparable inhibitory potency relative to GS3 and ritonavir.

TABLE 1
Properties of pyridine-containing compounds

| | 110 nm band^{a} | Kₛ^{b} | k_{fast}^{c} | ΔTₘ^{d} | IC₅₀^{e} |
|---|---|---|---|---|---|
| | (Fe²⁺) | (μM) | (s⁻¹) | (° C.) | (μM) |
| pyridine | − | 4500 ± 400 | 0.5 ± 0.1 | 0 | 4000 ± 500 |
| aminoethylpyridine | − | 2500 ± 200 | 0.6 ± 0.1 | −0.1 | 5000 ± 600 |
| 5 | − | 10 ± 1^{f} | 0.8 ± 0.1 | −0.2 | 1000 ± 150 |
| | | 4400 ± 500^{f} | | | |
| 4 | + | 105 ± 10 | 20 ± 2 | −0.2 | 75 ± 5 |
| 11 | + | 10 ± 1 | 4.0 ± 0.2 | −2 ± 0.4 | 30 ± 3 |
| 15a | ++ | 0.9 ± 0.1 | 1.7 ± 0.2 | −1 ± 0.2 | 0.52 ± 0.05 |
| 15b | ++ | 0.5 ± 0.1 | 8.0 ± 1.5 | 0.2 | 0.21 ± 0.04 |
| GS3^{g} | +++ | 0.025 | 7.0 | 5.1 | 0.13 |
| ritonavir^{g} | +++ | 0.051 | 1.4 | 2.7 | 0.55 |

^{a}Appearance and magnitude of the 442 nm peak in the absorbance spectrum of ligand-bound ferrous CYP3A4.
^{b}Spectral dissociation constant.
^{c}Rate constant for the fast phase of the binding reaction measured at saturating ligand concentrations.
^{d}Ligand-dependent changes in the melting temperature of CYP3A4.
^{e}Concentration required for half-maximal inactivation of recombinant CYP3A4.
^{f}Values derived from the two site saturation fit to a titration curve
^{g}Determined previously (Sevrioukova and Poulos, 2010, Proc Natl Acad Sci USA 107: 18422-7; Sevrioukova and Poulos, 2013, J Med Chem 56: 3733-41).

The binding reactions of CYP3A4 with pyridine, aminoethylpyridine and 5 were monophasic, with rate constants of 0.5-0.8 s⁻¹. In contrast, association with 4, 11 and 15a-b proceeded in two phases. The rate constant for the fast phase (k_{fast}) was the highest for 4 and the lowest for 15a (20 and 1.7 s⁻¹, respectively; Table 1). Notably, k_{fast} for 15a was close to that of ritonavir (1.4 s⁻¹) (Sevrioukova and Poulos, 2010, Proc Natl Acad Sci USA 107:18422-7) and nearly 5-fold lower than the respective value for 15b and GS3. Although not wishing to be bound by any particular theory, together, the spectral and kinetic data suggest that (i) the backbone elongation and addition of a side group complicate the binding reaction, (ii) the presence of a bulky, hydrophobic R² group increases the ligand affinity but slows down the association rate, and (iii) indole as a R² substituent promotes the complex formation with CYP3A4 to a higher degree than the hexane or phenyl rings.

### Melting Temperature and Inhibitor Potency

None of the investigated compounds increased the thermal stability of CYP3A4, as evidenced by the lack of feasible ligand-dependent changes in melting temperature (ΔTₘ; Table 1). CYP3A4 Tₘ was slightly increased upon complex formation with 15b but decreased in the presence of other compounds, most notably for the 11- and 15a-bound forms (by 1-2° C.). This is in contrast to ritonavir and its analogs, whose ligation to CYP3A4 increases Tₘ by 2-7° C. (Sevrioukova and Poulos, 2013, J Med Chem 56:3733-41; Sevrioukova and Poulos, 2013, Biochemistry 52:4474-81).

The inhibitory potency of the investigated compounds on the BFC debenzylation activity of CYP3A4 was found to correlate with the binding affinity (Table 1). The IC₅₀ values were the highest for pyridine, aminoethylpyridine and 5 (1-4 mM), intermediate for 4 and 11(75 and 30 μM, respectively), and the lowest for 15a and 15b (0.5 and 0.2 μM, respectively). Importantly, IC50s for 15a and 15b were close to those for ritonavir and GS3 (0.55 and 0.13 μM, respectively) (Sevrioukova and Poulos, 2013, J Med Chem 56:3733-41). Thus, addition of only one aromatic side group and tert-butyloxycarbonyl (Boc) as a terminal moiety transformed compound 5, a very weak ligand, into a potent CYP3A4 inactivator.

Crystal Structures of the 15a- and 15b-bound CYP3A4

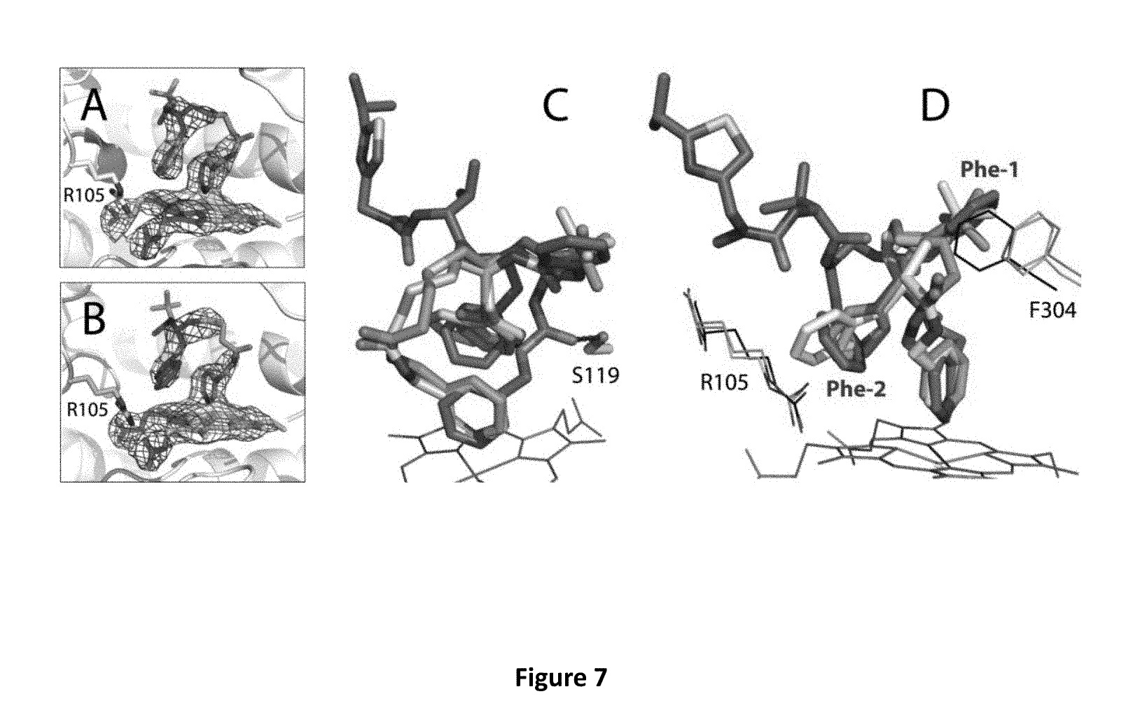

CYP3A4 was successfully co-crystallized with 4, 15a and 15b. Compound 11 dissociated from CYP3A4 during crystallization. Crystal structures of CYP3A4 ligated to 15a and 15b were determined to 2.60 and 2.76 Å, respectively (FIG. 13). Both compounds bind to the active site and ligate to the heme iron through the pyridine nitrogen (FIG. 7). The

GS3-bound model (PDB ID 4I4H) was used for comparative analysis because this analog is most similar to the investigated compounds. Superposition of the 15a-, 15b- and GS3-bound structures reveals that 15a and 15b rotate around the pyridine nitrogen by ca. 180° (FIG. 7C) to place the phenyl/indole rings between the heme-ligating pyridine and Arg105 guanidinium group (P2 site), right where the Phe-2 side group of GS3 is positioned. Most strikingly, the terminal Boc groups are in a similar position and displace Phe304 and the I-helix to the same extent as Phe-1 of GS3 does (FIG. 7D). Since the phenyl and indole moieties are oriented differently than GS3 Phe-2, which is nearly parallel to the pyridine and Arg105 guanidine groups and closer to the heme plane, the 15a-b conformations are less optimal for hydrophobic, π-π and π-cation interactions with the heme, pyridine and the Arg105 guanidine, respectively. Another important dissimilarity that may decrease affinity of 15a-b is that their binding mode disallows H-bonding with Ser119 (FIG. 7C). Nevertheless, the comparable K_{S} and IC₅₀ values for 15b and GS3 imply that the larger and more electron-rich indole ring may partially compensate for the less favorable orientation by establishing new or more extensive Van der Waals contacts with the neighboring Arg105, Ala370, heme and pyridine.

### Modification of Surface Residues in CYP3A4 for Improvement of Crystal Diffraction

Wild type CYP3A4 willingly co-crystallizes with 4, but similar to other ligand-bound forms, diffraction data of this complex did not exceed 2.5 Å. At this resolution level, electron density for 4 was discontinuous and the ligand association mode could not be accurately determined. To overcome this problem, surface mutations were introduced that help form intermolecular contacts in the crystal lattice and improve X-ray diffraction power without affecting the CYP3A4 ligand binding and metabolism. Using a Surface Entropy Reduction prediction (SERp) server (http://services.mbi.ucla.edu/SER/; Goldschmidt et al., 2007, Protein Sci 16:1569-76), several surface peptides containing clusters of bulky charged residues (Arg, Lys and/or Glu) were identified (Table 2). One cluster with a high score, Lys282/G1u283/G1u285, is part of the surface loop that is disordered in all crystal structures reported to date. This site was mutated and variant K282A/E285A, which was better expressed in E. coli, associated with 4 similarly to the wild type, and produced crystals with lower mosaicity and higher diffraction power. This mutant was used for determination of the CYP3A4-4 complex structure.

TABLE 2
High entropy residues in CYP3A4 identified by a Surface Entropy
Reduction prediction (SERp) server (http://services.mbi.ucla.edu/SER/;
Goldschmidt et al., 2007, Protein Sci 16: 1569-76).

| Cluster | Residues | SERp score^{a} | Proposed Mutation |
|---|---|---|---|
| 1 | ⁴²¹KKNK⁴²⁴ | 6.51 | K421A/K422A/K424A |
| 2 | ²⁸²KETESHKA²⁸⁹ | 6.3 | K282A/E263A/E285A |
| 3 | ⁴⁶⁹KETQ⁴⁷² | 5.26 | K469A/E470A |
| 4 | ⁴⁸⁶EK⁴⁸⁷ | 5.09 | E486A/K487A |
| 5 | ³⁷⁸KKDVE³⁸² | 5.07 | K378A/K379A |

^{a}The highest score is assigned to a cluster with the highest probability to enhance crystallizability upon mutation.

### Crystal Structures of the CYP3A4-4 Complexes

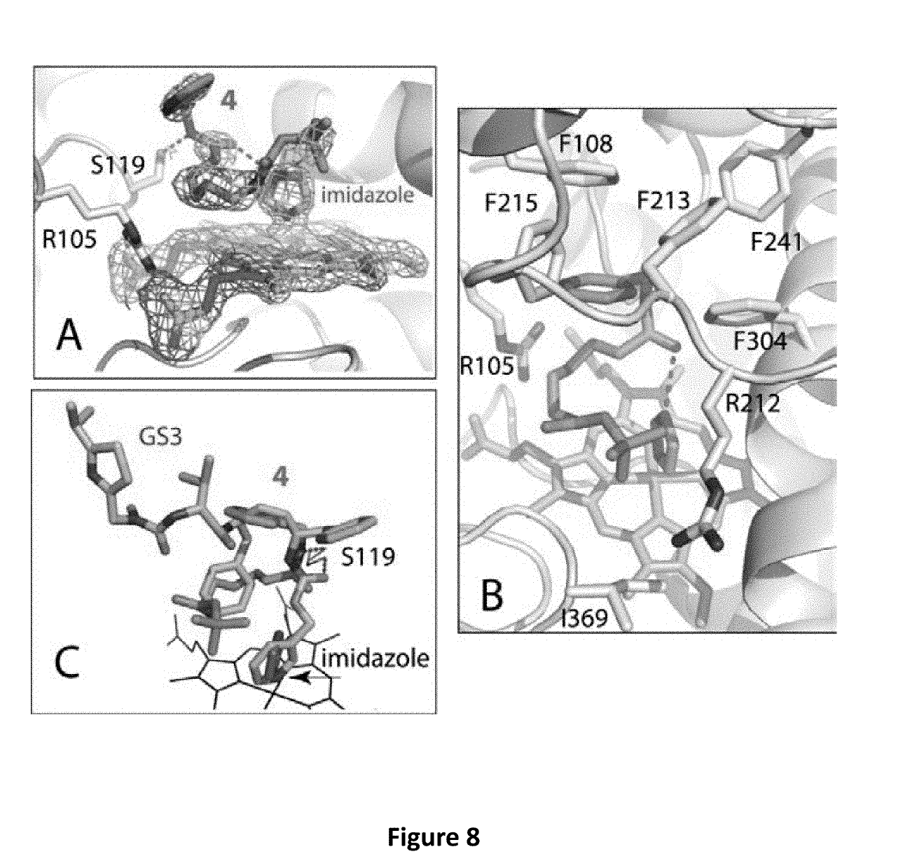

Co-crystals of CYP3A4 K282A/E285A with 4 were grown under two different conditions that led to two distinct modes of ligand binding. The first complex was crystallized in the presence of imidazole and contained both compound 4 and imidazole in the active site (FIG. 8A). In this structure, determined to 1.93 Å, imidazole ligates to the heme iron and 4 binds nearby. Such a ligand arrangement is stabilized by two hydrogen bonds formed between the carbonyl oxygen and amide nitrogen of 4 and the imidazole nitrogen and the Ser119 hydroxyl group, respectively. The pyridine moiety of 4 is adjacent but not embedded into the P1 pocket. Through hydrophobic interactions with Phe108, Phe213, Phe215 and Phe340 (FIG. 8B), the pyridine ring reinforces the phenylalanine cluster, a unique feature of CYP3A4 (Williams et al., 2004, Science 305:683-6). The aliphatic linker of 4 forms Van der Waals contacts with the Arg105 side chain, whereas the Boc group is placed at the P2 site and forms hydrophobic and Van der Waals interactions with the Ile369-Ala370 peptide and the Arg212 side chain. Thus, when the space near the heme iron is occupied, the flexible compound 4 adopts a conformation that allows H-bonding to Ser119 and optimizes hydrophobic interactions mediated by the Boc and pyridine moieties. It is not clear though why the pyridine of 4 does not insert into the P1 pocket as Boc of 15a-b (FIG. 7D) and Phe-2 of GS3 and other ritonavir analogs do (FIG. 8C) (Sevrioukova and Poulos, 2010, Proc Natl Acad Sci USA 107:18422-7; Sevrioukova and Poulos, 2013, J Med Chem 56:3733-41; Sevrioukova and Poulos, 2013, Biochemistry 52:4474-81). Although not wishing to be bound by any particular theory, the observed orientation may be preferable because it prevents steric clashing with Phe304 and the I-helix displacement. Alternatively, insertion into the P1 cavity may require some force (e.g., a push), which the flexible core of non-ligated 4 cannot provide.

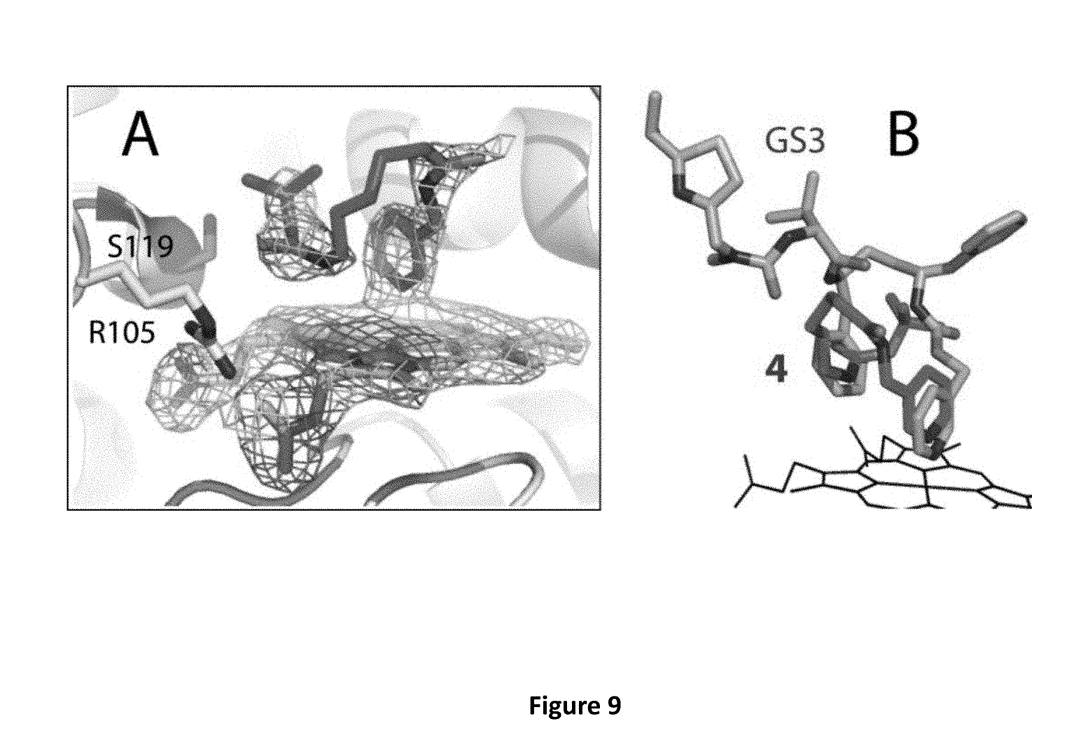

The X-ray structure of the CYP3A4-4 complex formed in the absence of imidazole was determined to 2.25 Å. In this structure, 4 is directly ligated to the heme iron via the pyridine nitrogen and, similar to 15a-b, rotates by ˜180° relative to GS3 (FIG. 9). The flexible linker bends to allow the end portion occupy the P2 site. Although not wishing to be bound by any particular theory, this result and the lack of H-bonds between 4 and the protein suggest once again that hydrophobic interactions, especially at the P2 site, are dominant and define the ligand binding mode.

### Rational Design of CYP3A4 Inhibitors Leads to Simpler, Potent Compounds

The data obtained with the first set of rationally designed compounds support the proposed pharmacophore model for a CYP3A4-specific inhibitor and emphasize the importance of pharmacophoric determinants II, III and V (FIG. 3), as well as the dominant role of hydrophobic forces at the P1 and P2 sites during the ligand-binding process. In particular, with this initial set, it was confirmed that (i) the flexible backbone enables ligands to better fit into the CYP3A4 active site and optimize interactions mediated by the side and terminal moieties; (ii) the P2 site is occupied by ligands first, and interactions at this site are dominant and define the overall ligand binding mode; (iii) the P1 cavity is the second preferable site that ligands tend to occupy, even if it leads to the I-helix distortion; and (iv) H-bonding interactions with Ser119 are established when possible to stabilize the CYP3A4-ligand complex.

There were also new findings that provided additional insights into the ligand binding process. First, pyridine and its short-chain aliphatic derivative, aminoethylpyridine (FIG. 4), were found to be very weak CYP3A4 inactivators (Table 1). This means that ligation of the pyridine nitrogen to the heme iron is weak and dissociable but becomes strong and virtually irreversible when the pyridine ring becomes part of the larger ligands, especially those capable of occupying the P2 and P1 sites. Second, based on the K_{S} and IC₅₀ values for 11, 15a and 15b (Table 1) and the inability of 11 to co-crystallize with CYP3A4, aromaticity rather than hydrophobicity of the R² substituent was found to stabilize the protein-ligand complex and drastically improve affinity and inhibitory potency. Third, the structural data demonstrate that the P1 site can be occupied by the terminal group if R¹ is absent. Although not wishing to be bound by any particular theory, the fact that none of the R¹-lacking compounds markedly affected CYP3A4 Tₘ suggests, however, that Boc-mediated interactions at the P1 site are sub-optimal and cannot provide the binding energy on the same scale as phenyl substituents of R¹ do (e.g., in ritonavir and GS3). Fourth, there was no correlation between ΔTₘ and IC₅₀ for the investigated compounds (Table 1) but, as observed for other ritonavir analogs (Sevrioukova and Poulos, 2013, J Med Chem 56:3733-41; Sevrioukova and Poulos, 2013, Biochemistry 52:4474-81), the inhibitory potency was proportional to the 442 nm absorption of the ferrous ligand-bound species. Thus, during ligand screening, the latter parameter may predict strong CYP3A4 inactivators more reliably than ΔTₘ. Finally, two of the rationally designed compounds, 15a and 15b, are less structurally complex than GS3 and ritonavir but inhibit CYP3A4 with a comparable, sub-micromolar potency.

### Example 2

### High Affinity CYP3A4 Inhibitors

The data presented herein describes high affinity CYP3A4 inhibitors.

### Synthesis of Starting Materials

### Synthesis of N-Isopropyl-L-Cysteine

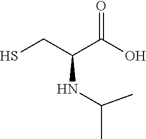

To a solution of L-cysteine (2.0 g, 16.5 mmol) in MeOH, sodium cyanoborohydride (1.03 g, 16.5 mmol) was slowly added. To this mixture, acetone (9.58 g, 165 mmol) was added and the reaction was allowed to stir at room temperature attached to a bubbler for 18-42 hrs. On completion, the precipitate was filtered, washed with MeOH, and dried in vacuo affording the pure product as a white powder in 25% yield (0.69g). ESI-MS (TOF MS ES+) m/zcalculated for C₆H₁₃NO₂S[M+Na]⁺: 186.057; found: 186.007

### Synthesis of N-Cyclopentyl-L-Cysteine

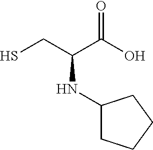 [SMILES: O=C(O)[C@H](CS)NC1CCCC1]

To a solution of L-cysteine (2.0 g, 16.5 mmol) in MeOH, sodium cyanoborohydride (1.03 g, 16.5 mmol) was slowly added. To this mixture, cyclopentanone (13.88 g, 165 mmol) was added and the reaction was allowed to stir at room temperature attached to a bubbler for 18-48 hrs. On completion, the precipitate was filtered, washed with MeOH, and dried in vacuo affording the pure product as a white powder in 27.5% yield (0.86 g). ESI-MS (TOF MS ES+) m/z calculated for C₈H₁₅NO₂S[M+Na]⁺: 212.072; found: 212.007

### Synthesis of N-Phenylcysteine

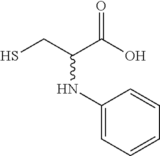 [SMILES: O=C(O)C(CS)Nc1ccccc1]

N-phenylcysteine was synthesized as described in Lee, G. H., Pak, C. S., & Lee, H. W. (1988) Synthesis of N-Phenylcysteine, Bulletin of the Korean Chemical Society, 9(1), 25-27; and Park, J. D., & Kim, D. H. (2002) Cysteine derivatives as inhibitors for carboxypeptidase A: Synthesis and structure-activity relationships, J Med Chem, 45(4), 911-918.

The product was obtained as an orange solid in a 20-50% yield. ESI-MS (TOF MS ES+) m/zcalculated for C₉H₁₁NO₂S[M+H]⁺: 198.058; found: 197.987/198.1810 (overlap)

### Synthesis of N-Boc-Trp-OTs

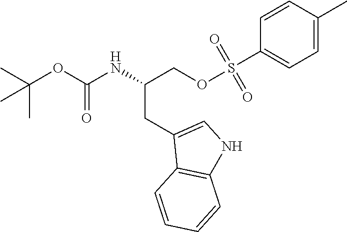 [SMILES: Cc1ccc(S(=O)(=O)OC[C@H](Cc2c[nH]c3ccccc23)NC(=O)OC(C)(C)C)cc1]

N-Boc-Trp-OTs was synthesized as described previously in Kauret. al. J Med Chem, 2013. This method was also applied to both Phe and Gly, with phenylalinol and Boc-ethanolamine as starting materials, respectively.

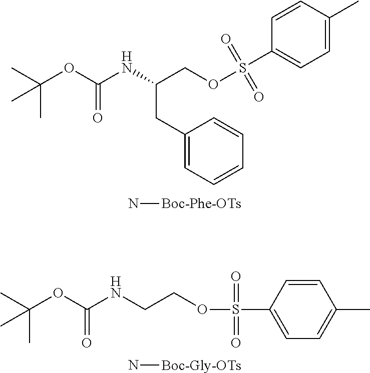

### Synthesis of 01ERS083

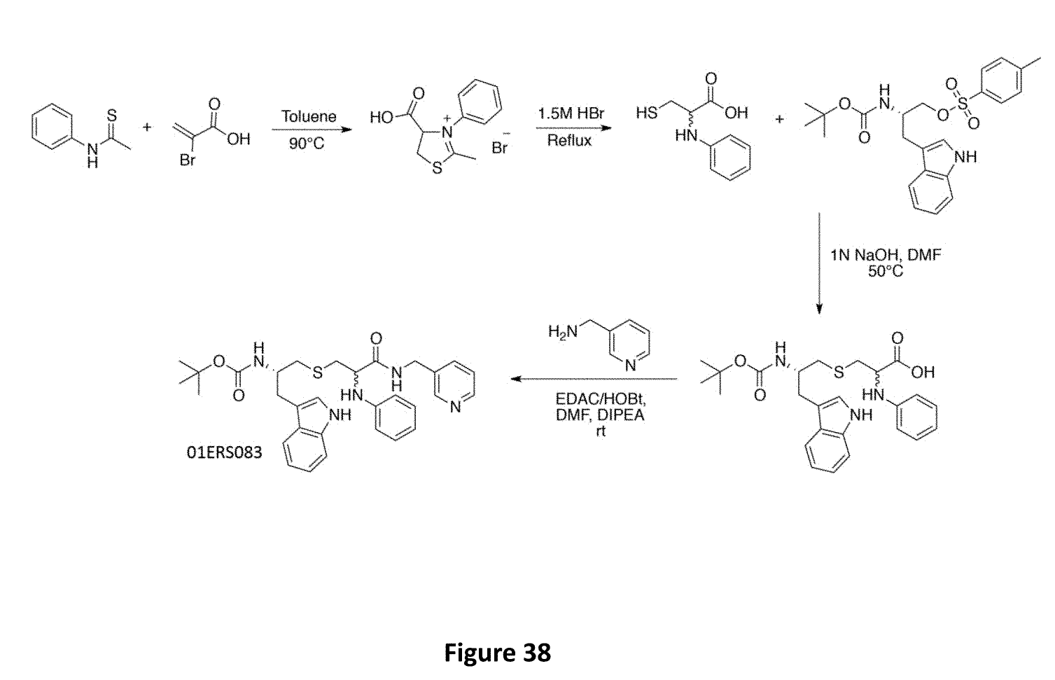

The schematic of 01ERS083 synthesis is shown in FIG. 38.

### Synthesis of N-BOC-Trp-S-N-Ph-Cys

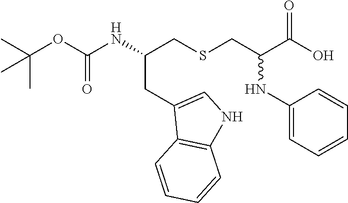 [SMILES: CC(C)(C)OC(=O)N[C@H](CSCC(Nc1ccccc1)C(=O)O)Cc1c[nH]c2ccccc12]

To a solution of N-BOC-Trp-OTs (0.15 g, 0.34 mmol) in DMF, N-phenylcysteine (0.115 g (0.42 mmol) was added. To the mixture, 1 N NaOH was added and the reaction was allowed to stir at 50° C. for 18 hrs. The reaction was cooled to room temperature and concentrated in vacuo. The crude product was used for the next step without further purification. ESI-MS (TOF MS ES+) m/zcalculated for C₂₅H₃₁N₃O₄S[M+Na]⁺: 492.193; found: 492.097

### Synthesis of 01ERS083 (N-BOC-Trp-S-N-Ph-Cys-CONH-Me-Pyr)

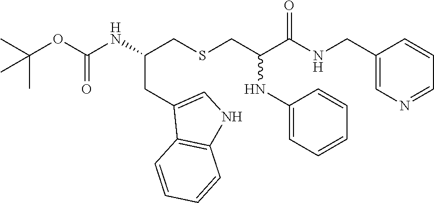 [SMILES: CC(C)(C)OC(=O)N[C@H](CSCC(Nc1ccccc1)C(=O)NCc1cccnc1)Cc1c[nH]c2ccccc12]

To a solution of N-BOC-Trp-S-N-Ph-Cys (0.15 g, 0.32 mmol) in DMF, EDAC·HCl (1.5 eq), HOBt hydrate (1.5 eq), and 3-(aminomethyl)pyridine (1.5 eq) were added. To the mixture, DIPEA (3 eq) was added and the reaction was allowed to stir at room temperature for 18 hrs. Upon completion, the solvent was evaporated and diluted with EtOAc and saturated NaHCO₃. The organic layers were extracted, washed with water and brine, and dried over MgSO₄. The crude product was then purified by column chromatography (95:5 EtOAc/MeOH). The pure product was obtained as a white fluffy solid in a 30% yield (0.053g). ESI-MS (TOF MS ES+) m/z calculated for C₃₁H₃₇N₅O₃S [M+H]⁺: 560.268; found: 560.127.

### Synthesis of 01ERS089

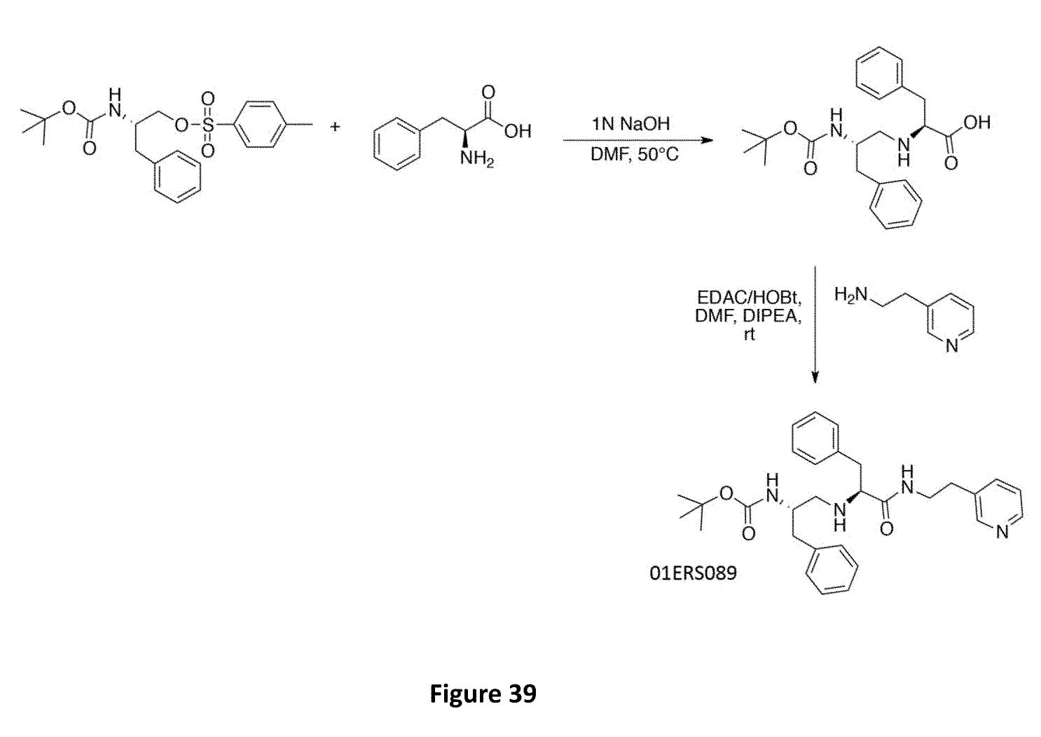

The schematic of 01ERS089 synthesis is shown in FIG. 39.

### Step 1: Synthesis of N-Boc-Phe-N-Phe-CONH-Et-Pyr

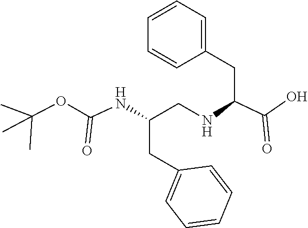 [SMILES: CC(C)(C)OC(=O)N[C@H](CN[C@@H](Cc1ccccc1)C(=O)O)Cc1ccccc1]

To a solution of N-BOC-Phe-OTs (0.2 g, 0.49 mmol) in DMF, L-phenylalanine (0.1 g (0.6 mmol) was added. To the mixture, 1 N NaOH was added and the reaction was allowed to stir at 50° C. for 18 hrs. The reaction was cooled to room temperature and concentrated in vacuo. The crude product was used for the next step without further purification. ESI-MS (TOF MS ES+) m/z calculated for C₂₃H₃₀N₂O₄[M+H]⁺: 399.227; found: 399.125.

### Step 2: Synthesis of N-BOC-Trp-S-N-Ph-Cys-CONH-Et-Pyr (01ERS089)

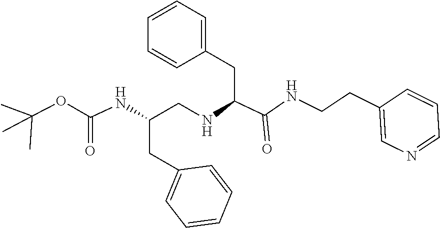 [SMILES: CC(C)(C)OC(=O)N[C@H](CN[C@@H](Cc1ccccc1)C(=O)NCCc1cccnc1)Cc1ccccc1]

To a solution of N-Boc-Phe-N-Phe-CONH-Et-Pyr (0.11 g, 0.28 mmol) in DMF, EDAC·HCl (1.5 eq), HOBt hydrate (1.5 eq), and 3-(2-aminoethyl)pyridine (1.5 eq) were added. To the mixture, DIPEA (3 eq) was added and the reaction was allowed to stir at room temperature for 18 hrs. Upon completion, the solvent was evaporated and diluted with EtOAc and saturated NaHCO₃. The organic layers were extracted, washed with water and brine, and dried over MgSO₄. The crude product was then purified by column chromatography (95:5 EtOAc/MeOH). The pure product was obtained as a yellow film in a trace yield. ESI-MS (TOF MS ES+) m/z calculated for C₃₀H₃₈N₄O₃[M+H]⁺: 503.301; found: 503.197.

### Synthesis of 01ERS087

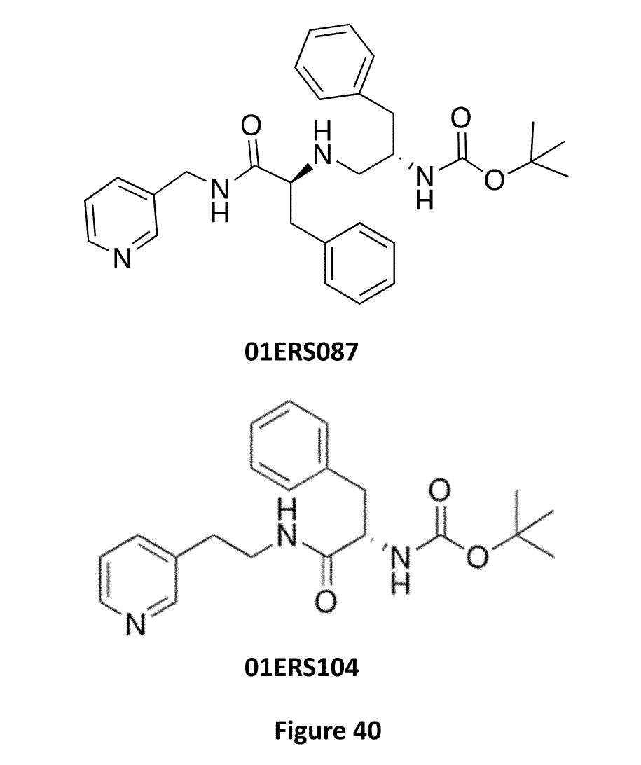

01ERS087 (FIG. 40) was synthesized using the protocol of 01ERS089 except 3-(2-aminoethyl)pyridine was replaced with 3-(aminomethyl)pyridine to produce the methyl pyridine derivative (01ERS087) also in trace yield. ESI-MS (TOF MS ES+) m/z calculated for C₂₉H₃₆N₄O₃[M+H]⁺: 489.285; found: 489.154

### Alternative Synthetic Method for Phe-Phe or Trp-Trp

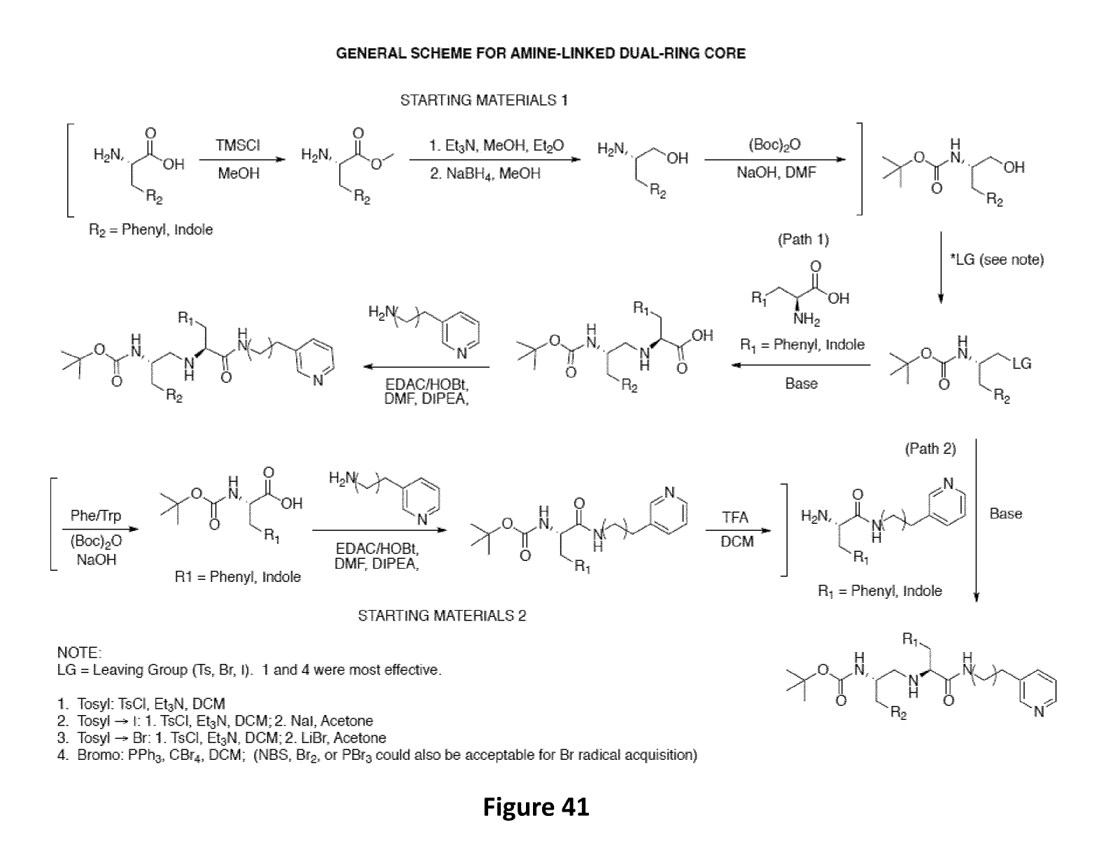

To improve yield (Phe-Phe) and completion (Trp-Trp) the following alternative synthetic routes can be utilized, such as the route shown in FIG. 41.

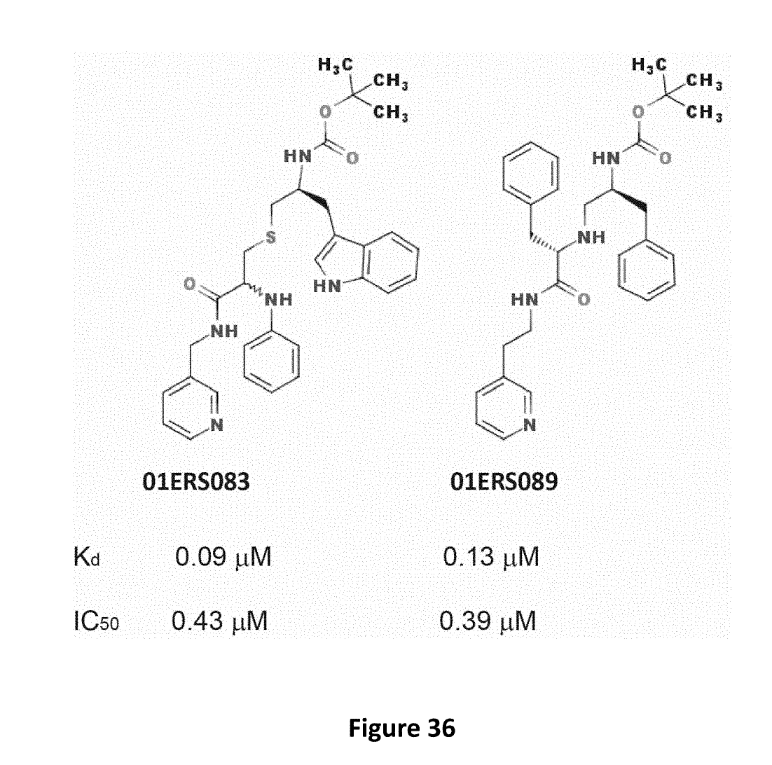

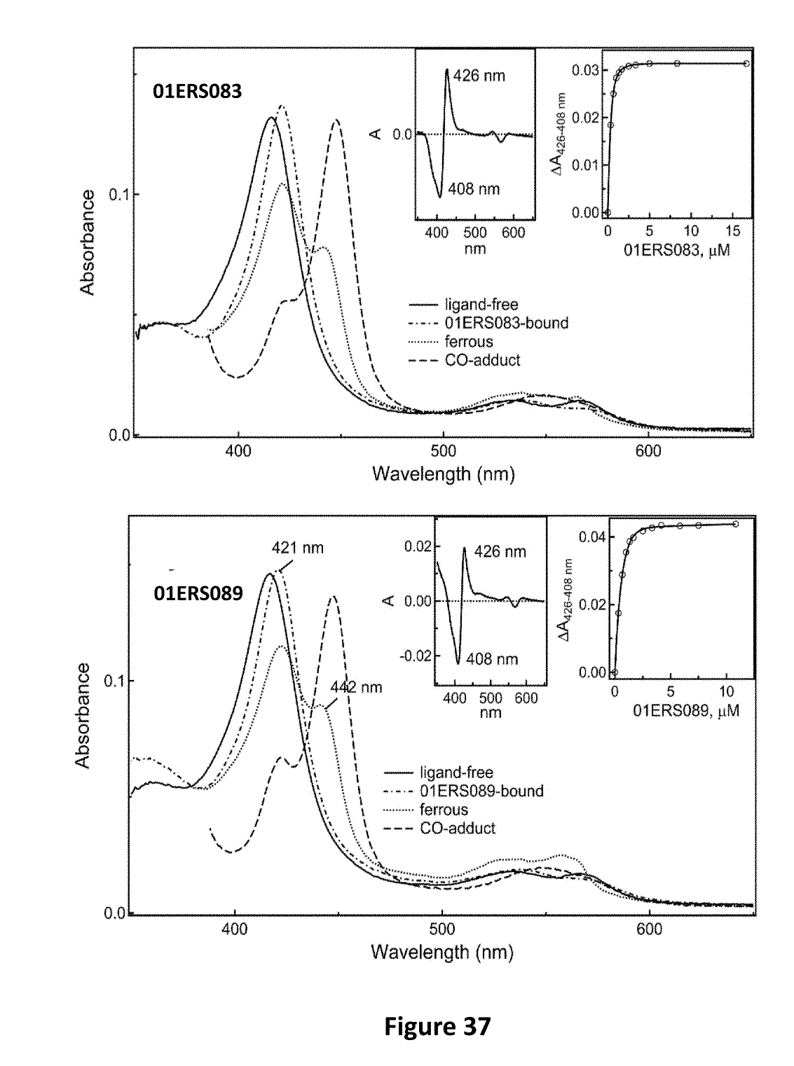

Compounds 01ERS083 and 01ERS089 (FIG. 36) have a strong binding affinity to CYP3A4 having a K_{d} of 0.09 μM and 0.13 μM, respectively. The inhibitory potency of 01ERS083 and 01ERS089 on the 7-benzyloxy-4-(trifluoromethyl)coumarin (BFC) O-debenzylation activity of CYP3A4 was evaluated fluorometrically in a reconstituted system with rat cytochrome P450 reductase. 01ERS083 and 01ERS089 exhibited IC₅₀ of 0.43 μM and 0.39 μM, respectively. Further, both 01ERS083 and 01ERS089 induce spectral changes in CYP3A4 (FIG. 37).

### Modifications of 01ERS Compounds

The compounds described herein may be modified to improve activity, decrease molecule size and improve solubility, as would be understood by one of ordinary skill in the art. For example, diminishing R³ may achieve a useful compound. Examples of these compounds include

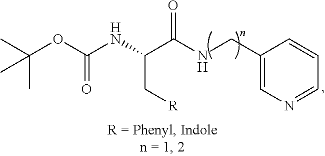

where R₁=R, R₂=Boc, and R₃=H

For example, 01ERS104 (FIG. 39) (N-Boc-Phe-CONH-et-pyr) can be synthesized as follows. To a solution of N-Boc-Phenylalanine (0.5 g, 1.9 mmol) in DMF, EDAC·HCl (1.5 eq), HOBt hydrate (1.5 eq), and 3-(2-aminoethyl)pyridine (1.5 eq) were added. To the mixture, DIPEA (3 eq) was added and the reaction was allowed to stir at room temperature for 18 hrs. Upon completion, the solvent was evaporated and diluted with EtOAc and saturated NaHCO₃. The organic layers were extracted, washed with water and brine, and dried over MgSO₄. The crude product was then purified by column chromatography (9:1 EtOAc/MeOH). The pure product was obtained as a white solid/clear gum in an 89% yield (0.624g). ESI-MS (TOF MS ES+) m/z calculated for C₂₁H₂₇N₃O₃[M+H]⁺: 370.212; found: 370.133.

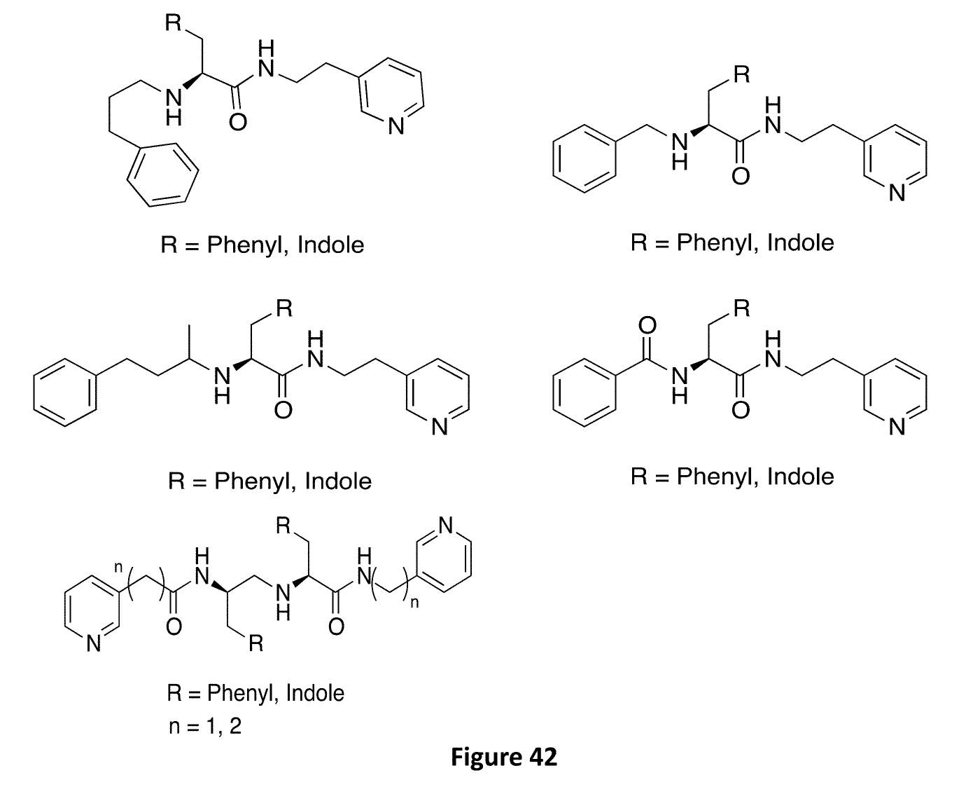

Other useful CYP3A4 inhibitors include those as shown in FIG. 42.

The disclosures of each and every patent, patent application, and publication cited herein are hereby incorporated herein by reference in their entirety. While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound of Formula (I): 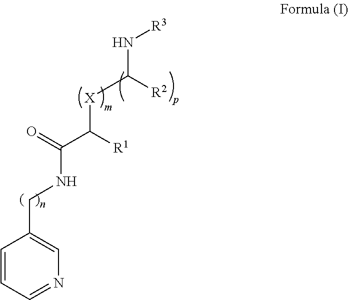
wherein in Formula (I):
R¹ and R² are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, substitued -(C₁-C₆)alkyl-heteroaryl, and N(R⁴)(R⁵);
R³ is selected from the group consisting of H, -C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substituted -(C₁-C₆)alkyl-heteroaryl and -C(═O)R⁶, wherein the alkyl group is optionally substituted;
R⁴ and R⁵ are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substitued-(C₁-C₆)alkyl-heteroaryl;
R⁶ is selected from the group consisting of phenyl, substituted phenyl, -(C₁-C₆)alkyl-phenyl, substitued -(C₁-C₆) alkyl-phenyl, -(C₁-C₆)alkyl-heteroaryl, substitued -(C₁-C₆)alkyl-heteroarly, and OR⁷;
R⁷ is selected from the group consisting of H, -C₁-C₆ alkyl, substituted C₁-C₆ alkyl,aryl, cycloalkyl phenyl, substituted phenyl, heteroaryl, substituted heteroaryl, heterocyclyl, substituted heterocyclyl, -(C₁-C₆)alkyl-phenyl, substituted -(C₁-C₆)alkyl-aryl, substituted -(C₁-C₆)alkyl-aryl, -(C₁-C₆)alkyl-carbocyclic, -(C₁-C₆)alkyl-heteroaryl, substitued -(C₁-C₆)alkyl-heteroaryl;
each occurrence of X is independently selected from the group consisting of CH₂, NH, S, and O;
m is an integer from 0 to 3;
n is an integer from 0 to 3; and
p is an integer from 0 to 1;
a salt or solvate, and any combinations thereof

2. The compound of claim 1, wherein R⁷ is selected from the group consisting of H, benzyl, and aniline.

3. The compound of claim 1, wherein R² is selected from the group consisting of H, -(C₁-C₆)alkylene-carbocyclic, -(C₁-C₆)alkylene-phenyl, -(C₁-C₆)alkylene-substituted phenyl, -(C₁-C₆)alkylene-heteroaryl, or -(C₁-C₆)alkylene-substituted heteroaryl.

4. The compound of claim 1, wherein R⁷ is -C₁-C₆ alkyl.

5. The compound of claim 1, wherein m is 1.

6. The compound of claim 1, wherein n is 1.

7. The compound of claim 1, wherein X is S.

8. The compound of claim 1, wherein the compound is selected from the group consisting of: 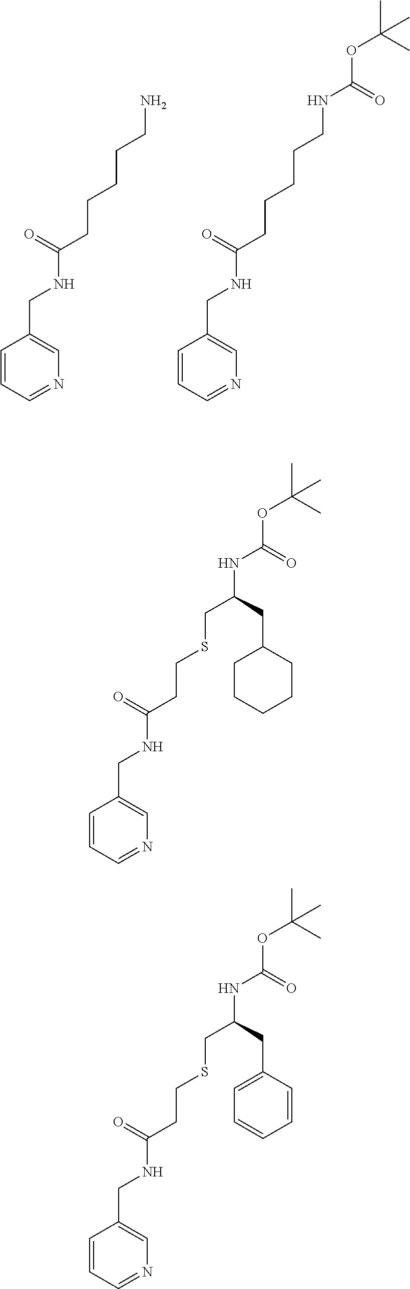 [SMILES: NCCCCCC(=O)NCc1cccnc1 ; CC(C)(C)OC(=O)NCCCCCC(=O)NCc1cccnc1 ; CC(C)(C)OC(=O)N[C@H](CSCCC(=O)NCc1cccnc1)CC1CCCCC1 ; CC(C)(C)OC(=O)N[C@H](CSCCC(=O)NCc1cccnc1)Cc1ccccc1] 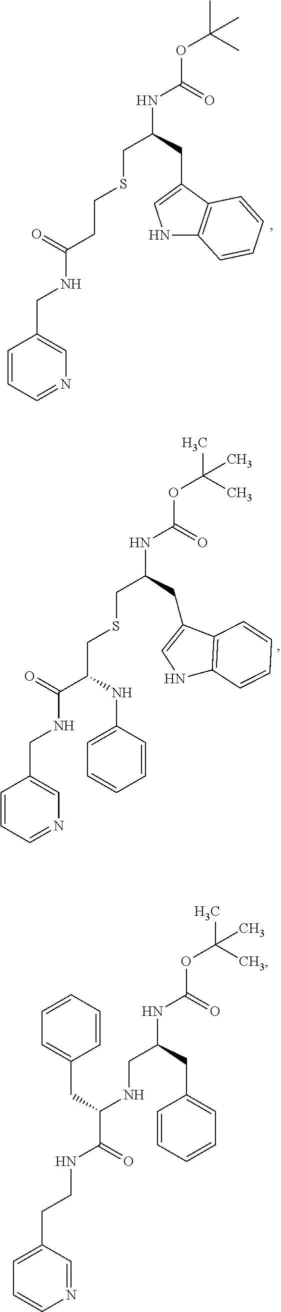 [SMILES: CC(C)(C)OC(=O)N[C@H](CSCCC(=O)NCc1cccnc1)Cc1c[nH]c2ccccc12 ; CC(C)(C)OC(=O)N[C@H](CSC[C@H](Nc1ccccc1)C(=O)NCc1cccnc1)Cc1c[nH]c2ccccc12 ; CC(C)(C)OC(=O)N[C@H](CN[C@@H](Cc1ccccc1)C(=O)NCCc1cccnc1)Cc1ccccc1] 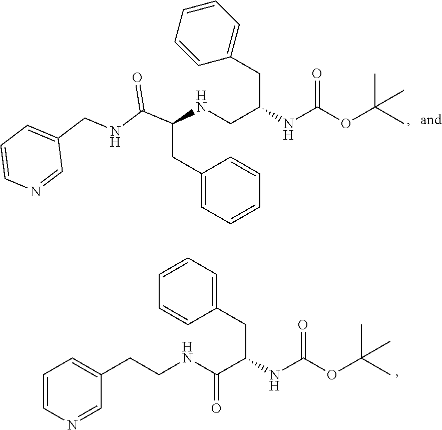 [SMILES: CC(C)(C)OC(=O)N[C@H](CN[C@@H](Cc1ccccc1)C(=O)NCc1cccnc1)Cc1ccccc1 ; CC(C)(C)OC(=O)N[C@@H](Cc1ccccc1)C(=O)NCCc1cccnc1]
a salt or solvate thereof, and any combinations thereof.

9. The compound of claim 1, wherein the compound is selected from the group consisting of 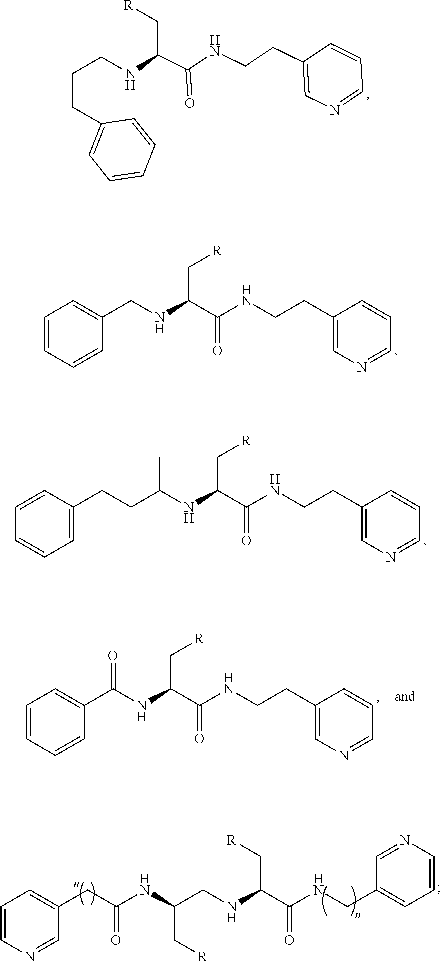
wherein each occurrence of R is selected from the group consisting of phenyl and indole; and
wherein each occurrence of n is independently 1 or 2.

10. A composition comprising a compound of claim 1.

11. The composition of claim 10, wherein the composition further comprises a pharmaceutically acceptable carrier.

12. A method of inhibiting CYP3A4 in a subject in need thereof, the method comprising administering to the subject a therapeutically effective amount of a composition comprising at least one compound of Formula (I): 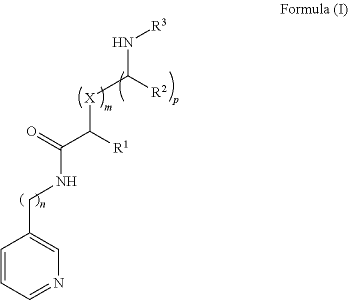
wherein in Formula (I):
R¹ and R² are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl,-(C₁-C₆)alkyl-phenyl, substituted -(C₁-C₆)alkyl-phenyl, -(C₁-C₆)alkyl-carbocyclic, -(C₁-C₆)alkyl-heteroaryl, substitued -(C₁-C₆)alkyl-heteroaryl, and N(R⁴)(R⁵);
R³ is selected from the group consisting of H, -C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substituted -(C₁-C₆)alkyl-heteroaryl and -C(═O)R⁶, wherein the alkyl group is optionally substituted;
R⁴ and R⁵ are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, -(C₁-C₆)alkyl-aryl, substituted -(C₁-C₆)alkyl-aryl, -(C₁-C₆)alkyl-phenyl, substituted -(C₁-C₆)alkyl-phenyl, -(C₁-C₆)alkyl-carbocyclic, -(C₁-C₆)alkyl-heteroaryl, and substitued-(C₁-C₆)alkyl-heteroaryl;
R⁶ is selected from the group consisting of phenyl, substituted phenyl, -(C₁-C₆)alkyl-phenyl, substitued -(C₁-C₆) alkyl-phenyl, -(C₁-C₆)alkyl-heteroaryl, substitued -(C₁-C₆)alkyl-heteroarly, and OR⁷;
R⁷ is selected from the group consisting of H, -C₁-C₆ alkyl, substituted C₁-C₆ alkyl,aryl, cycloalkyl phenyl, substituted phenyl, heteroaryl, substituted heteroaryl, heterocyclyl, substituted heterocyclyl, -(C₁-C₆)alkyl-phenyl, substituted -(C₁-C₆)alkyl-aryl, substituted -(C₁-C₆)alkyl-aryl, -(C₁-C₆)alkyl-carbocyclic, -(C₁-C₆)alkyl-heteroaryl, substitued -(C₁-C₆)alkyl-heteroaryl;
each occurrence of X is independently selected from the group consisting of CH₂, NH, S, and O;
m is an integer from 0 to 3;
n is an integer from 0 to 3; and
p is an integer from 0 to 1;
a salt or solvate, and any combinations thereof

13. The method of claim 12, wherein R¹ is selected from the group consisting of H, benzyl, and aniline.

14. The method of claim 12, wherein R² is selected from the group consisting of H, -(C₁-C₆)alkylene-carbocyclic, -(C₁-C₆)alkylene-phenyl, -(C₁-C₆)alkylene-substituted phenyl, -(C₁-C₆)alkylene-heteroaryl, or -(C₁-C₆)alkylene-substituted heteroaryl.

15. The method of claim 12, wherein R⁷ is -C₁-C₆ alkyl.

16. The method of claim 12, wherein m is 1.

17. The method of claim 12, wherein n is 1.

18. The method of claim 12, wherein X is S.

19. The method of claim 12, wherein the compound is selected from the group consisting of: 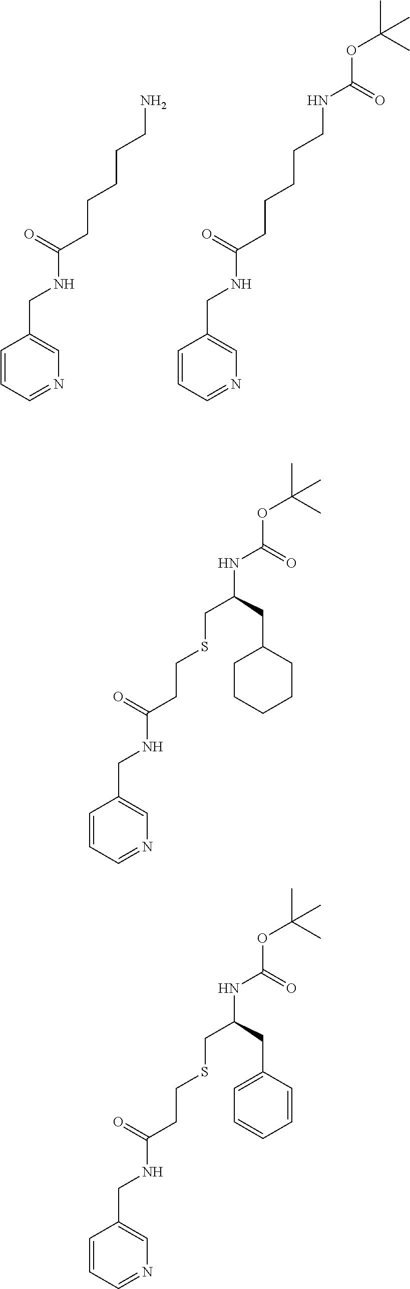 [SMILES: NCCCCCC(=O)NCc1cccnc1 ; CC(C)(C)OC(=O)NCCCCCC(=O)NCc1cccnc1 ; CC(C)(C)OC(=O)N[C@H](CSCCC(=O)NCc1cccnc1)CC1CCCCC1 ; CC(C)(C)OC(=O)N[C@H](CSCCC(=O)NCc1cccnc1)Cc1ccccc1] 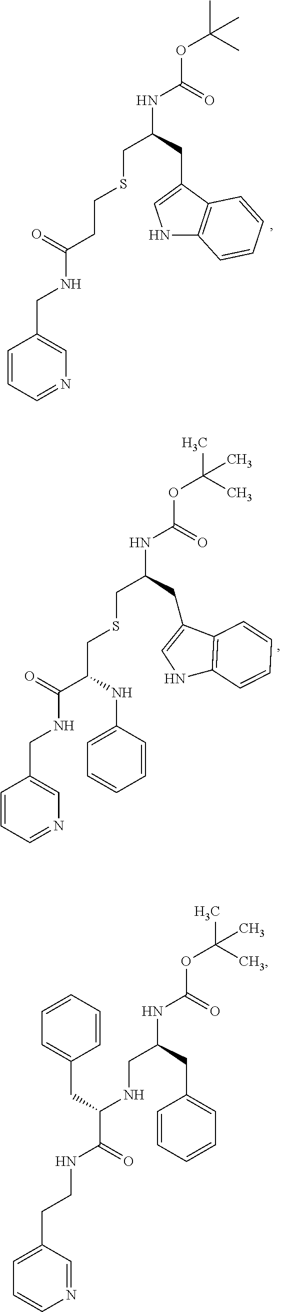 [SMILES: CC(C)(C)OC(=O)N[C@H](CSCCC(=O)NCc1cccnc1)Cc1c[nH]c2ccccc12 ; CC(C)(C)OC(=O)N[C@H](CSC[C@H](Nc1ccccc1)C(=O)NCc1cccnc1)Cc1c[nH]c2ccccc12 ; CC(C)(C)OC(=O)N[C@H](CN[C@@H](Cc1ccccc1)C(=O)NCCc1cccnc1)Cc1ccccc1] 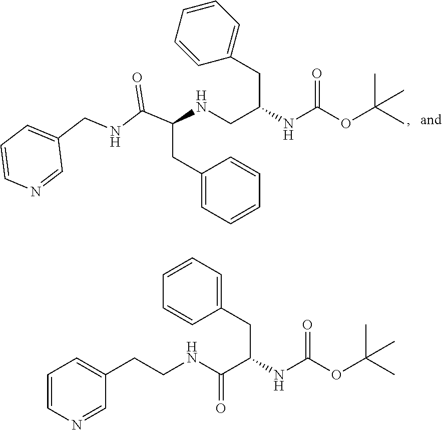 [SMILES: CC(C)(C)OC(=O)N[C@H](CN[C@@H](Cc1ccccc1)C(=O)NCc1cccnc1)Cc1ccccc1 ; CC(C)(C)OC(=O)N[C@@H](Cc1ccccc1)C(=O)NCCc1cccnc1]
a salt or solvate thereof, and any combinations thereof.

20. The method of claim 12, wherein the method further comprises administering to the subject at least one additional therapeutic agent.

21. The method of claim 20, wherein the therapeutic agent is an antiviral agent.

22. The method of claim 20, wherein the composition and the additional therapeutic agent are co-administered.

23. The method of claim 22, wherein the composition and the additional therapeutic agent are co-formulated.

24. The method of claim 20, wherein the therapeutic agent is a protease inhibitor.
